(19) United States
(12) Patent Application Publication
(10) Pub. No.: US 2018/0055869 A1
(43) Pub. Date: Mar. 1, 2018

(54) COMPOSITIONS AND METHODS FOR MODULATING RNA

(71) Applicant: Translate Bio MA, Inc., Cambridge, MA (US)

(72) Inventor: Fatih Ozsolak, Boston, MA (US)

(73) Assignee: Translate Bio MA, Inc., Cambridge, FL (US)

(21) Appl. No.: 15/550,112

(22) PCT Filed: Feb. 12, 2016
(86) PCT No.: PCT/US2016/017826

§ 371 Date: Aug. 10, 2017

Related U.S. Application Data
(60) Provisional application No. 62/115,766, filed on Feb. 13, 2015.

(51) Int. Cl. A61K 31/7088; C12N 15/113

(52) U.S. Cl. CPC A61K 31/7088; C12N 15/113; C12N 2310/3231; C12N 2310/315; C12N 2310/317; C12N 2310/11

(57) ABSTRACT

Aspects of the invention relate to methods for increasing gene expression in a targeted manner. In some embodiments, methods are provided for increasing expression of a gene expressed in a liver cell. In some embodiments, methods and compositions are provided that are useful for posttranscriptionally altering protein and/or RNA levels in a targeted manner. Aspects of the invention disclosed herein provide methods and compositions that are useful for protecting RNAs from degradation (e.g., exonuclease mediated degradation).

### CROSS-REFERENCE TO RELATED APPLICATIONS

This application claims the benefit under 35 U.S.C. §119(e) of U.S. Provisional Application No. 62/115,766, entitled “COMPOSITIONS AND METHODS FOR MODULATING RNA”, filed Feb. 13, 2015, the contents of which are incorporated herein by reference in its entirety.

### FIELD OF THE INVENTION

The invention relates to oligonucleotide based compositions, as well as methods of using oligonucleotide based compositions for modulating nucleic acids.

### BACKGROUND OF THE INVENTION

A considerable portion of human diseases can be treated by selectively altering protein and/or RNA levels of disease-associated transcription units (noncoding RNAs, protein-coding RNAs or other regulatory coding or noncoding genomic regions). Methods for inhibiting the expression of genes are known in the art and include, for example, antisense, RNAi and miRNA mediated approaches. Such methods may involve blocking translation of mRNAs or causing degradation of target RNAs. However, limited approaches are available for increasing the expression of genes.

### SUMMARY OF THE INVENTION

Aspects of the invention disclosed herein relate to methods and compositions useful for modulating nucleic acids. In some embodiments, methods and compositions provided herein are useful for protecting RNAs (e.g., RNA transcripts) from degradation (e.g., exonuclease mediated degradation). In some embodiments, the protected RNAs are present outside of cells. In some embodiments, the protected RNAs are present in cells. In some embodiments, methods and compositions are provided that are useful for posttranscriptionally altering protein and/or RNA levels in a targeted manner. In some embodiments, methods disclosed herein involve reducing or preventing degradation or processing of targeted RNAs thereby elevating steady state levels of the targeted RNAs. In some embodiments, methods disclosed herein may also or alternatively involve increasing translation or increasing transcription of targeted RNAs, thereby elevating levels of RNA and/or protein levels in a targeted manner.

Aspects of the invention relate to a recognition that certain RNA degradation is mediated by exonucleases. In some embodiments, exonucleases may destroy RNA from its 3′ end and/or 5′ end. Without wishing to be bound by theory, in some embodiments, it is believed that one or both ends of RNA can be protected from exonuclease enzyme activity by contacting the RNA with oligonucleotides (oligos) that hybridize with the RNA at or near one or both ends, thereby increasing stability and/or levels of the RNA. The ability to increase stability and/or levels of a RNA by targeting the RNA at or near one or both ends, as disclosed herein, is surprising in part because of the presence of endonucleases (e.g., in cells) capable of destroying the RNA through internal cleavage. Moreover, in some embodiments, it is surprising that a 5′ targeting oligonucleotide is effective alone (e.g., not in combination with a 3′ targeting oligonucleotide or in the context of a pseudocircularization oligonucleotide) at stabilizing RNAs or increasing RNA levels because in cells, for example, 3′ end processing exonucleases may be dominant (e.g., compared with 5′ end processing exonucleases). However, in some embodiments, 3′ targeting oligonucleotides are used in combination with 5′ targeting oligonucleotides, or alone, to stabilize a target RNA.

In some embodiments, where a targeted RNA is protein-coding, increases in steady state levels of the RNA result in concomitant increases in levels of the encoded protein. Thus, in some embodiments, oligonucleotides (including 5′-targeting, 3′-targeting and pseudocircularization oligonucleotides) are provided herein that when delivered to cells increase protein levels of target RNAs. In some embodiments is notable that not only are target RNA levels increased but the resulting translation products are also increased. In some embodiments, this result is surprising in part because of an understanding that for translation to occur ribosomal machinery requires access to certain regions of the RNA (e.g., the 5′ cap region, start codon, etc.) to facilitate translation.

In some embodiments, where the targeted RNA is non-coding, increases in steady state levels of the non-coding RNA result in concomitant increases activity associated with the non-coding RNA. For example, in instances where the non-coding RNA is an miRNA, increases in steady state levels of the miRNA may result in increased degradation of mRNAs targeted by the miRNA.

In some embodiments, oligonucleotides are provided with chemistries suitable for delivery, hybridization and stability within cells to target and stabilize RNA transcripts. Furthermore, in some embodiments, oligonucleotide chemistries are provided that are useful for controlling the pharmacokinetics, biodistribution, bioavailability and/or efficacy of the oligonucleotides.

In some embodiments, oligonucleotides, methods, kits and compositions are provided for increasing gene expression of a gene selected from: FXN, THRB, HAMP, APOA1 and NR1H4, e.g., by increasing stability of RNA transcripts expressed from the gene. In some embodiments, oligonucleotides, methods, kits and compositions are provided for increasing gene expression in a cell, such as a liver cell in a human subject.

Aspects of the invention relate to methods of increasing stability of a THRB or NR1H4 RNA transcript in a cell. In some embodiments, methods provided herein involve delivering to a cell one or more oligonucleotides disclosed herein that stabilize a THRB or NR1H4 RNA transcript. In some embodiments, the methods involve delivering to a cell a first stabilizing oligonucleotide that targets a 5′ region of the RNA transcript and a second stabilizing oligonucleotide that targets the 3′ region of the RNA transcript. In some embodiments, the first stabilizing oligonucleotide is covalently linked with the second stabilizing oligonucleotide. In some embodiments, the first stabilizing oligonucleotide comprises a region of complementarity that is complementary with the RNA transcript at a position within 10 nucleotides of the first transcribed nucleotide at the 5′ end of the RNA transcript. In some embodiments, the RNA transcript comprises a 5′-methylguanosine cap, and the first stabilizing oligonucleotide comprises a region of complementarity that is complementary with the RNA transcript at a position within 10 nucleotides of the nucleotide immediately internal to the 5′-methylguanosine cap. In some embodiments, the second stabilizing oligonucleotide comprises a region of complementarity that is complementary with the RNA transcript at a position within 250 nucleotides of the 3′ end of the RNA transcript. In some embodiments, the RNA transcript comprises a 3′-poly(A) tail, and the second stabilizing oligonucleotide comprises a region of complementarity that is complementary with the RNA transcript at a position within 100 nucleotides of the polyadenylation junction of the RNA transcript. In some embodiments, the region of complementarity of the second stabilizing oligonucleotide is immediately adjacent to or overlapping the polyadenylation junction of the RNA transcript. In some embodiments, the cell is in vitro. In some embodiments, the cell is in vivo. In some embodiments, the second stabilizing oligonucleotide comprises a region of complementarity that is complementary with the RNA transcript at a position within the 3′-poly(a) tail. In some embodiments, the second stabilizing oligonucleotide comprises a region comprising 5 to 15 pyrimidine (e.g., thymine) nucleotides.

Other aspects of the invention relate to methods of increasing stability of an RNA transcript in a human liver cell (e.g., a human hepatocyte), such as a liver cell in a human subject. In some embodiments, methods provided herein involve delivering to a human liver cell (e.g., a human hepatocyte) one or more oligonucleotides disclosed herein that stabilize an RNA transcript. In some embodiments, methods provided herein involve delivering to a human subject one or more oligonucleotides disclosed herein in an amount effective to stabilize an RNA transcript in a liver cell of the subject. In some embodiments, the methods involve delivering to a human liver cell (e.g., a human hepatocyte) a first stabilizing oligonucleotide that targets a 5′ region of the RNA transcript and a second stabilizing oligonucleotide that targets the 3′ region of the RNA transcript. In some embodiments, the methods involve delivering to a human subject first stabilizing oligonucleotide that targets a 5′ region of the RNA transcript and a second stabilizing oligonucleotide that targets the 3′ region of the RNA transcript, in an amount effective to stabilize the RNA transcript in a liver cell of the subject. In some embodiments, the first stabilizing oligonucleotide is covalently linked with the second stabilizing oligonucleotide. In some embodiments, the first stabilizing oligonucleotide comprises a region of complementarity that is complementary with the RNA transcript at a position within 10 nucleotides of the first transcribed nucleotide at the 5′ end of the RNA transcript. In some embodiments, the RNA transcript comprises a 5′-methylguanosine cap, and the first stabilizing oligonucleotide comprises a region of complementarity that is complementary with the RNA transcript at a position within 10 nucleotides of the nucleotide immediately internal to the 5′-methylguanosine cap. In some embodiments, the second stabilizing oligonucleotide comprises a region of complementarity that is complementary with the RNA transcript at a position within 250 nucleotides of the 3′ end of the RNA transcript. In some embodiments, the RNA transcript comprises a 3′-poly(A) tail, and the second stabilizing oligonucleotide comprises a region of complementarity that is complementary with the RNA transcript at a position within 100 nucleotides of the polyadenylation junction of the RNA transcript. In some embodiments, the region of complementarity of the second stabilizing oligonucleotide is immediately adjacent to or overlapping the polyadenylation junction of the RNA transcript. In some embodiments, the cell is in vitro. In some embodiments, the cell is in vivo. In some embodiments, the second stabilizing oligonucleotide comprises a region of complementarity that is complementary with the RNA transcript at a position within the 3′-poly(a) tail. In some embodiments, the second stabilizing oligonucleotide comprises a region comprising 5 to 15 pyrimidine (e.g., thymine) nucleotides.

Further aspects of the invention relate to methods of treating a condition or disease associated with decreased levels of an RNA transcript in a liver cell (e.g., a hepatocyte) of a human subject. In some embodiments, the methods involve administering an oligonucleotide to the subject.

In some embodiments of the foregoing methods, the RNA transcript is an mRNA, non-coding RNA, long non-coding RNA, miRNA, snoRNA or any other suitable transcript.

In some embodiments, the RNA transcript is an mRNA expressed from a gene selected from the group consisting of: THRB, HAMP, APOA1 and NR1H4. In some embodiment, the mRNA is a synthetic mRNA (e.g., a synthetic mRNA containing one or more modified ribonucleotides).

In some aspects of the invention, an oligonucleotide is provided that comprises a region of complementarity that is complementary with at least 5 contiguous nucleotides of an RNA transcript expressed from a THRB or NR1H4 gene, in which the nucleotide at the 3′-end of the region of complementary is complementary with a nucleotide within 10 nucleotides of the transcription start site of the RNA transcript. In some embodiments, the oligonucleotide comprises nucleotides linked by at least one modified internucleoside linkage or at least one bridged nucleotide. In some embodiments, the oligonucleotide is 8 to 50 or 9 to 20 nucleotides in length.

In some aspects of the invention, an oligonucleotide is provided that comprises two regions of complementarity each of which is complementary with at least 5 contiguous nucleotides of an RNA transcript expressed from a THRB or NR1H4 gene, in which the nucleotide at the 3′-end of the first region of complementary is complementary with a nucleotide within 100 nucleotides of the transcription start site of the RNA transcript and in which the second region of complementarity is complementary with a region of the RNA transcript that ends within 300 nucleotides of the 3′-end of the RNA transcript.

In some aspects of the invention, an oligonucleotide is provided that comprises the general formula 5′-X₁-X₂-3′, in which X₁ comprises 5 to 20 nucleotides that have a region of complementarity that is complementary with at least 5 contiguous nucleotides of an RNA transcript expressed from a THRB or NR1H4 gene, in which the nucleotide at the 3′-end of the region of complementary of X₁ is complementary with the nucleotide at the transcription start site of the RNA transcript; and X₂ comprises 1 to 20 nucleotides. In some embodiments, the RNA transcript has a 7-methylguanosine cap at its 5′-end. In some embodiments, the RNA transcript has a 7-methylguanosine cap, and wherein the nucleotide at the 3′-end of the region of complementary of X₁ is complementary with the nucleotide of the RNA transcript that is immediately internal to the 7-methylguanosine cap. In some embodiments, at least the first nucleotide at the 5′-end of X₂ is a pyrimidine complementary with guanine. In some embodiments, the second nucleotide at the 5′-end of X₂ is a pyrimidine complementary with guanine. In some embodiments, X₂ comprises the formula 5′-Y₁-Y₂-Y₃-3′, in which X₂ forms a stem-loop structure having a loop region comprising the nucleotides of Y₂ and a stem region comprising at least two contiguous nucleotides of Y₁ hybridized with at least two contiguous nucleotides of Y₃. In some embodiments, Y₁, Y₂ and Y₃ independently comprise 1 to 10 nucleotides. In some embodiments, Y₃ comprises, at a position immediately following the 3′-end of the stem region, a pyrimidine complementary with guanine. In some embodiments, Y₃ comprises 1-2 nucleotides following the 3′ end of the stem region. In some embodiments, the nucleotides of Y₃ following the 3′ end of the stem region are DNA nucleotides. In some embodiments, the stem region comprises 2-3 LNAs. In some embodiments, the pyrimidine complementary with guanine is cytosine. In some embodiments, the nucleotides of Y₂ comprise at least one adenine. In some embodiments, Y₂ comprises 3-4 nucleotides. In some embodiments, the nucleotides of Y₂ are DNA nucleotides. In some embodiments, Y₂ comprises 3-4 DNA nucleotides comprising at least one adenine nucleotide. It should be appreciated that one or more modified nucleotides (e.g., 2′-O-methyl, LNA nucleotides) may be present in Y₂. In some embodiments, X₂ comprises a region of complementarity that is complementary with at least 5 contiguous nucleotides of the RNA transcript that do not overlap the region of the RNA transcript that is complementary with the region of complementarity of X₁. In some embodiments, the region of complementarity of X₂ is within 100 nucleotides of a polyadenylation junction of the RNA transcript. In some embodiments, the region of complementarity of X₂ is complementary with the RNA transcript immediately adjacent to or overlapping the polyadenylation junction of the RNA transcript. In some embodiments, X₂ further comprises at least 2 consecutive pyrimidine nucleotides complementary with adenine nucleotides of the poly(A) tail of the RNA transcript. In some embodiments, the region of complementarity of X₂ is within the poly(a) tail. In some embodiments, the region of complementarity of X₂ comprises 5 to 15 pyrimidine (e.g., thymine) nucleotides. In some embodiments, the RNA transcript is an mRNA, non-coding RNA, long non-coding RNA, miRNA, snoRNA or any other suitable RNA transcript. In some embodiments, the RNA transcript is an mRNA transcript, and X₂ comprises a region of complementarity that is complementary with at least 5 contiguous nucleotides in the 3′-UTR of the transcript.

In some aspects of the invention, an oligonucleotide is provided that is 10 to 50 or 9 to 50 or 9 to 20 nucleotides in length and that has a first region complementary with at least 5 consecutive nucleotides of the 5′-UTR an mRNA transcript expressed from a THRB or NR1H4 gene, and a second region complementary with at least 5 consecutive nucleotides of the 3′-UTR, poly(A) tail, or overlapping the polyadenylation junction of the mRNA transcript. In some embodiments, the first of the at least 5 consecutive nucleotides of the 5′-UTR is within 10 nucleotides of the 5′-methylguanosine cap of the mRNA transcript. In some embodiments, the second region is complementary with at least 5 consecutive nucleotides overlapping the polyadenylation junction. In some embodiments, the second region is complementary with at least 5 consecutive nucleotides of the poly(a) tail. In some embodiments, the second region comprises 5 to 15 pyrimidine (e.g., thymine) nucleotides. In some embodiments, the oligonucleotide further comprises 2-20 nucleotides that link the 5′ end of the first region with the 3′ end of the second region. In some embodiments, the oligonucleotide further comprises 2-20 nucleotides that link the 3′ end of the first region with the 5′ end of the second region. In some embodiments, the oligonucleotide is 10 to 50 or 9 to 50 or 9 to 20 nucleotides in length.

In some aspects of the invention, an oligonucleotide is provided that comprises the general formula 5′-X₁-X₂-3′, in which X₁ comprises 2 to 20 pyrimidine nucleotides that form base pairs with adenine; and X₂ comprises a region of complementarity that is complementary with at least 3 contiguous nucleotides of a poly-adenylated transcript expressed from a THRB or NR1H4 gene, wherein the nucleotide at the 5′-end of the region of complementary of X₂ is complementary with the nucleotide of the RNA transcript that is immediately internal to the poly-adenylation junction of the RNA transcript. In some embodiments, X₁ comprises 2 to 20 thymidines or uridines.

In some aspects of the invention, an oligonucleotide is provided that comprises a region of complementarity that is complementary with at least 5 contiguous nucleotides of an RNA transcript of a gene that is expressed in a liver cell (e.g., a human liver cell), in which the nucleotide at the 3′-end of the region of complementary is complementary with a nucleotide within 10 nucleotides of the transcription start site of the RNA transcript. In some embodiments, the oligonucleotide comprises nucleotides linked by at least one modified internucleoside linkage or at least one bridged nucleotide. In some embodiments, the oligonucleotide is 8 to 50 or 9 to 20 nucleotides in length.

In some aspects of the invention, an oligonucleotide is provided that comprises two regions of complementarity each of which is complementary with at least 5 contiguous nucleotides of an RNA transcript of a gene that is expressed in a liver cell (e.g., a human liver cell), in which the nucleotide at the 3′-end of the first region of complementary is complementary with a nucleotide within 100 nucleotides of the transcription start site of the RNA transcript and in which the second region of complementarity is complementary with a region of the RNA transcript that ends within 300 nucleotides of the 3′-end of the RNA transcript.

In some aspects of the invention, an oligonucleotide is provided that comprises the general formula 5′-X₁-X₂-3′, in which X₁ comprises 5 to 20 nucleotides that have a region of complementarity that is complementary with at least 5 contiguous nucleotides of an RNA transcript of a gene that is expressed in a liver cell (e.g., a human liver cell), in which the nucleotide at the 3′-end of the region of complementary of X₁ is complementary with the nucleotide at the transcription start site of the RNA transcript; and X₂ comprises 1 to 20 nucleotides. In some embodiments, the RNA transcript has a 7-methylguanosine cap at its 5′-end. In some embodiments, the RNA transcript has a 7-methylguanosine cap, and wherein the nucleotide at the 3′-end of the region of complementary of X₁ is complementary with the nucleotide of the RNA transcript that is immediately internal to the 7-methylguanosine cap. In some embodiments, at least the first nucleotide at the 5′-end of X₂ is a pyrimidine complementary with guanine. In some embodiments, the second nucleotide at the 5′-end of X₂ is a pyrimidine complementary with guanine. In some embodiments, X₂ comprises the formula 5′-Y₁-Y₂-Y₃-3′, in which X₂ forms a stem-loop structure having a loop region comprising the nucleotides of Y₂ and a stem region comprising at least two contiguous nucleotides of Y₁ hybridized with at least two contiguous nucleotides of Y₃. In some embodiments, Y₁, Y₂ and Y₃ independently comprise 1 to 10 nucleotides. In some embodiments, Y₃ comprises, at a position immediately following the 3′-end of the stem region, a pyrimidine complementary with guanine. In some embodiments, Y₃ comprises 1-2 nucleotides following the 3′ end of the stem region. In some embodiments, the nucleotides of Y₃ following the 3′ end of the stem region are DNA nucleotides. In some embodiments, the stem region comprises 2-3 LNAs. In some embodiments, the pyrimidine complementary with guanine is cytosine. In some embodiments, the nucleotides of Y₂ comprise at least one adenine. In some embodiments, Y₂ comprises 3-4 nucleotides. In some embodiments, the nucleotides of Y₂ are DNA nucleotides. In some embodiments, Y₂ comprises 3-4 DNA nucleotides comprising at least one adenine nucleotide. It should be appreciated that one or more modified nucleotides (e.g., 2′-O-methyl, LNA nucleotides) may be present in Y₂. In some embodiments, X₂ comprises a region of complementarity that is complementary with at least 5 contiguous nucleotides of the RNA transcript that do not overlap the region of the RNA transcript that is complementary with the region of complementarity of X₁. In some embodiments, the region of complementarity of X₂ is within 100 nucleotides of a polyadenylation junction of the RNA transcript. In some embodiments, the region of complementarity of X₂ is complementary with the RNA transcript immediately adjacent to or overlapping the polyadenylation junction of the RNA transcript. In some embodiments, X₂ further comprises at least 2 consecutive pyrimidine nucleotides complementary with adenine nucleotides of the poly(A) tail of the RNA transcript. In some embodiments, the region of complementarity of X₂ is within the poly(a) tail. In some embodiments, the region of complementarity of X₂ comprises 5 to 15 pyrimidine (e.g., thymine) nucleotides. In some embodiments, the RNA transcript is an mRNA, non-coding RNA, long non-coding RNA, miRNA, snoRNA or any other suitable RNA transcript. In some embodiments, the RNA transcript is an mRNA transcript, and X₂ comprises a region of complementarity that is complementary with at least 5 contiguous nucleotides in the 3′-UTR of the transcript. In some embodiments, the RNA transcript is an mRNA expressed from a gene selected from the group consisting of: THRB, HAMP, APOA1 and NR1H4. In some embodiments, X₂ comprises the sequence CC.

In some aspects of the invention, an oligonucleotide is provided that is 10 to 50 or 9 to 50 or 9 to 20 nucleotides in length and that has a first region complementary with at least 5 consecutive nucleotides of the 5′-UTR of an mRNA transcript of a gene that is expressed in a liver cell (e.g., a human liver cell), and a second region complementary with at least 5 consecutive nucleotides of the 3′-UTR, poly(A) tail, or overlapping the polyadenylation junction of the mRNA transcript. In some embodiments, the first of the at least 5 consecutive nucleotides of the 5′-UTR is within 10 nucleotides of the 5′-methylguanosine cap of the mRNA transcript. In some embodiments, the second region is complementary with at least 5 consecutive nucleotides overlapping the polyadenylation junction. In some embodiments, the second region is complementary with at least 5 consecutive nucleotides of the poly(a) tail. In some embodiments, the second region comprises 5 to 15 pyrimidine (e.g., thymine) nucleotides. In some embodiments, the oligonucleotide further comprises 2-20 nucleotides that link the 5′ end of the first region with the 3′ end of the second region. In some embodiments, the oligonucleotide further comprises 2-20 nucleotides that link the 3′ end of the first region with the 5′ end of the second region. In some embodiments, the oligonucleotide is 10 to 50 or 9 to 50 or 9 to 20 nucleotides in length.

In some aspects of the invention, an oligonucleotide is provided that comprises the general formula 5′-X₁-X₂-3′, in which X₁ comprises 2 to 20 pyrimidine nucleotides that form base pairs with adenine; and X₂ comprises a region of complementarity that is complementary with at least 3 contiguous nucleotides of a poly-adenylated RNA transcript of a gene that is expressed in a liver cell (e.g., a human liver cell), wherein the nucleotide at the 5′-end of the region of complementary of X₂ is complementary with the nucleotide of the RNA transcript that is immediately internal to the poly-adenylation junction of the RNA transcript. In some embodiments, X₁ comprises 2 to 20 thymidines or uridines.

In some embodiments, an oligonucleotide provided herein comprises at least one modified internucleoside linkage. In some embodiments, an oligonucleotide provided herein comprises at least one modified nucleotide. In some embodiments, at least one nucleotide comprises a 2′ O-methyl. In some embodiments, an oligonucleotide comprises at least one ribonucleotide, at least one deoxyribonucleotide, at least one 2′-fluoro-deoxyribonucleotides or at least one bridged nucleotide. In some embodiments, the bridged nucleotide is a LNA nucleotide, a cEt nucleotide or a ENA modified nucleotide. In some embodiments, each nucleotide of the oligonucleotide is a LNA nucleotide. In some embodiments, the nucleotides of the oligonucleotide comprise alternating deoxyribonucleotides and 2′-fluoro-deoxyribonucleotides, 2′-O-methyl nucleotides, or bridged nucleotides. In some embodiments, an oligonucleotide provided herein is mixmer. In some embodiments, an oligonucleotide provided herein is morpholino.

In some aspects of the invention, an oligonucleotide is provided that comprises a nucleotide sequence as set forth in Table 3. In some aspects of the invention, an oligonucleotide is provided that comprises a fragment of at least 8 nucleotides of a nucleotide sequence as set forth in Table 3.

In some aspects of the invention, a composition is provided that comprises a first oligonucleotide having 5 to 25 nucleotides linked through internucleoside linkages, and a second oligonucleotide having 5 to 25 nucleotides linked through internucleoside linkages, in which the first oligonucleotide is complementary with at least 5 consecutive nucleotides within 100 nucleotides of the 5′-end of an RNA transcript of a gene that is expressed in a liver cell (e.g., a human liver cell) and in which the second oligonucleotide is complementary with at least 5 consecutive nucleotides within 100 nucleotides of the 3′-end of an RNA transcript. In some embodiments, the first oligonucleotide and second oligonucleotide are joined by a linker that is not an oligonucleotide having a sequence complementary with the RNA transcript. In some embodiments, the linker is an oligonucleotide. In some embodiments, the linker is a polypeptide.

In some aspects of the invention, compositions are provided that comprise one or more oligonucleotides disclosed herein. In some embodiments, compositions are provided that comprise a plurality of oligonucleotides, in which each of at least 75% of the oligonucleotides comprise or consist of a nucleotide sequence as set forth in Table 3. In some embodiments, the oligonucleotide is complexed with a monovalent cation (e.g., Li+, Na+, K+, Cs+). In some embodiments, the oligonucleotide is in a lyophilized form. In some embodiments, the oligonucleotide is in an aqueous solution. In some embodiments, the oligonucleotide is provided, combined or mixed with a carrier (e.g., a pharmaceutically acceptable carrier). In some embodiments, the oligonucleotide is provided in a buffered solution. In some embodiments, the oligonucleotide is conjugated to a carrier (e.g., a peptide, steroid or other molecule). In some aspects of the invention, kits are provided that comprise a container housing the composition.

### BRIEF DESCRIPTION OF THE DRAWINGS

FIG. 1 is an illustration depicting exemplary oligo designs for targeting 3′ RNA ends. The first example shows oligos complementary to the 3′ end of RNA, before the polyA-tail. The second example shows oligos complementary to the 3′ end of RNA with a 5′ T-stretch to hybridize to a polyA tail. The sequences in FIG. 1 both correspond to SEQ ID NO: 122.

FIG. 2 is an illustration depicting exemplary oligos for targeting 5′ RNA ends. The first example shows oligos complementary to the 5′ end of RNA. The second example shows oligos complementary to the 5′ end of RNA, the oligo having 3′ overhang residues to create a RNA-oligo duplex with a recessed end. Overhang can include a combination of nucleotides including, but not limited to, C to potentially interact with a 5′ methylguanosine cap and stabilize the cap further. The sequences in FIG. 2 both correspond to SEQ ID NO: 122.

FIG. 3A is an illustration depicting exemplary oligos for targeting 5′ RNA ends and exemplary oligos for targeting 5′ and 3′ RNA ends. The example shows oligos with loops to stabilize a 5′ RNA cap or oligos that bind to a 5′ and 3′ RNA end to create a pseudo-circularized RNA. The sequence in FIG. 3A corresponds to SEQ ID NO: 122.

FIG. 3B is an illustration depicting exemplary oligo-mediated RNA pseudo-circularization. The illustration shows an LNA mixmer oligo binding to the 5′ and 3′ regions of an exemplary RNA. The sequence in FIG. 3B corresponds to SEQ ID NO: 123.

FIG. 4 is a photograph of two Western blots of APOA1 protein levels in primary mouse hepatocytes.

FIG. 5 is a graph showing human FXN mRNA levels in the Sarsero FRDA mouse model.

FIG. 6 is a graph showing a primary screen of human THRB end-targeting oligonucleotides. The three boxed 5′ oligonucleotides show an increase in THRB mRNA as singles. All oligonucleotides were used at a concentration of 10 μM.

FIG. 7A is a graph showing concentration-dependent upregulation of THRB with the 5′ end-targeting oligonucleotide, THRB-85 m01. The graph shows TRβ isoform 1-specific (exons 2-3) increases in pooled donors.

FIG. 7B is a graph showing concentration-dependent upregulation of THRB with the 5′ end-targeting oligonucleotide, THRB-85 m01. The graph shows all isoforms of TRβ (exons 10-11) in pooled donors.

FIG. 8 is a graph showing mRNA changes in a single donor after five day treatment with APOA1 or THRB (TRβ isoform 1-specific (exons 2-3)) lead or non-hit oligonucleotides.

FIG. 9A is a graph illustrating downstream gene analysis in 5 donor pooled primary human hepatocytes with T3 treatment.

FIG. 9B is a graph illustrating downstream gene analysis in single donor primary human hepatocytes with T3 treatment.

FIG. 10 is a graph illustrating downstream genes as a readout for TRβ upregulation in single donor primary human hepatocytes treated with THRB-85 m01 and T3.

FIG. 11 is a schematic illustrating the alignment of THRB-85 m01 to THRB using RaNA human hepatocyte RNASeq.

FIG. 12A is an illustration showing the detailed mechanism of the basal repression of THRB activity.

FIG. 12B is an illustration showing the detailed mechanism of the basal activation of THRB activity.

FIG. 13 is a schematic showing a comparison between TRα and TRβ.

FIG. 14 is a graph showing APOA1 and THRB mRNA after 5 day oligonucleotide treatment of mouse primary hepatocytes.

### DETAILED DESCRIPTION OF CERTAIN EMBODIMENTS OF THE INVENTION

Methods and compositions disclosed herein are useful in a variety of different contexts in which is it desirable to protect RNAs from degradation, including protecting RNAs inside or outside of cells. In some embodiments, methods and compositions are provided that are useful for posttranscriptionally altering protein and/or RNA levels in cells in a targeted manner. For example, methods are provided that involve reducing or preventing degradation or processing of targeted RNAs thereby elevating steady state levels of the targeted RNAs. In some embodiments, the stability of an RNA is increased by protecting one or both ends (5′ or 3′ ends) of the RNA from exonuclease activity, thereby increasing stability of the RNA.

In some embodiments, methods of increasing gene expression are provided. As used herein the term, “gene expression” refers generally to the level or representation of a product of a gene in a cell, tissue or subject. It should be appreciated that a gene product may be an RNA transcript or a protein, for example. An RNA transcript may be protein coding. An RNA transcript may be non-protein coding, such as, for example, a long non-coding RNA, a long intergenic non-coding RNA, a non-coding RNA, an miRNA, a small nuclear RNA (snRNA), or other functional RNA. In some embodiments, methods of increasing gene expression may involve increasing stability of a RNA transcript, and thereby increasing levels of the RNA transcript in the cell. Methods of increasing gene expression may alternatively or in addition involve increasing transcription or translation of RNAs. In some embodiments, other mechanisms of manipulating gene expression may be involved in methods disclosed herein.

In some embodiments, methods provided herein involve delivering to a cell (e.g., liver cell such as a human liver cell) one or more sequence specific oligonucleotides that hybridize with an RNA transcript at or near one or both ends, thereby protecting the RNA transcript from exonuclease mediated degradation. In embodiments where the targeted RNA transcript is protein-coding, increases in steady state levels of the RNA typically result in concomitant increases in levels of the encoded protein. In embodiments where the targeted RNA is non-coding, increases in steady state levels of the non-coding RNA typically result in concomitant increases activity associated with the non-coding RNA. In some embodiments, approaches disclosed herein based on regulating RNA levels and/or protein levels using oligonucleotides targeting RNA transcripts by mechanisms that increase RNA stability and/or translation efficiency may have several advantages over other types of oligos or compounds, such as oligonucleotides that alter transcription levels of target RNAs using cis or noncoding based mechanisms. For example, in some embodiments, lower concentrations of oligos may be used when targeting RNA transcripts in the cytoplasm as multiple copies of the target molecules exist. In contrast, in some embodiments, oligos that target transcriptional processes may need to saturate the cytoplasm and before entering nuclei and interacting with corresponding genomic regions, of which there are only one/two copies per cell, in many cases. In some embodiments, response times may be shorter for RNA transcript targeting because RNA copies need not to be synthesized transcriptionally. In some embodiments, a continuous dose response may be easier to achieve. In some embodiments, well defined RNA transcript sequences facilitate design of oligonucleotides that target such transcripts. In some embodiments, oligonucleotide design approaches provided herein, e.g., designs having sequence overhangs, loops, and other features facilitate high oligo specificity and sensitivity compared with other types of oligonucleotides, e.g., certain oligonucleotides that target transcriptional processes.

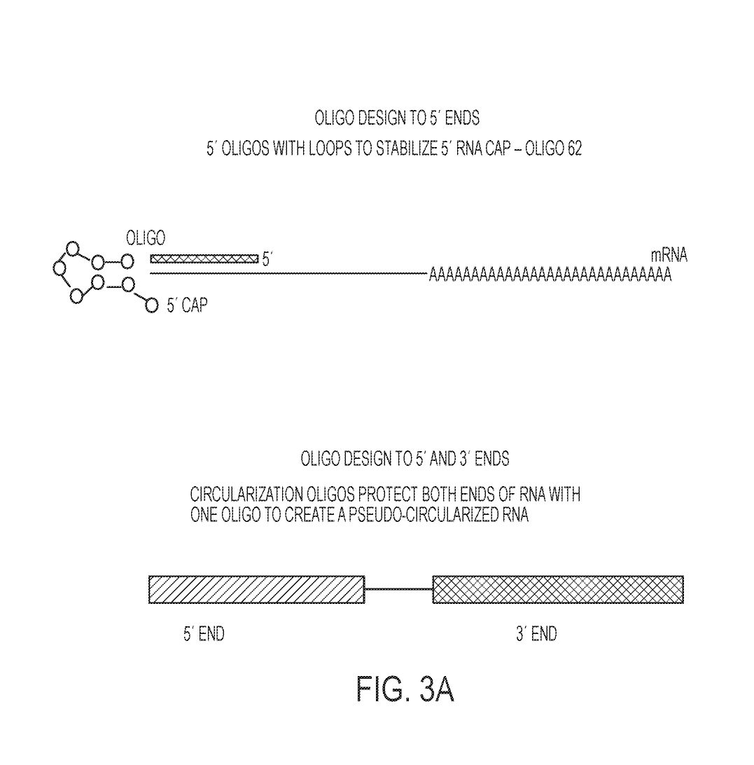

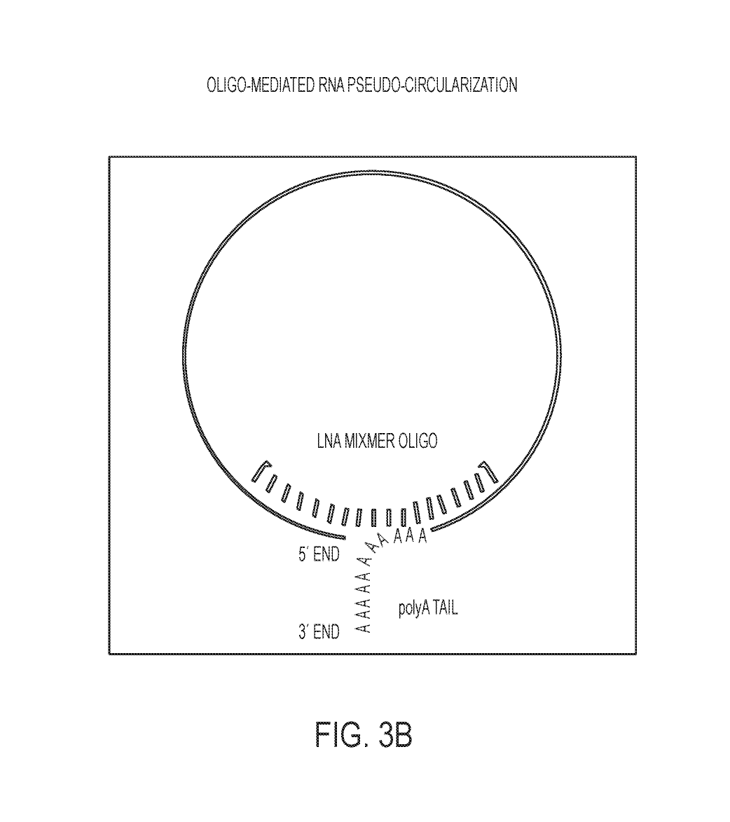

In some embodiments, methods provided herein involve use of oligonucleotides that stabilize an RNA by hybridizing at a 5′ and/or 3′ region of the RNA. In some embodiments, oligonucleotides that prevent or inhibit degradation of an RNA by hybridizing with the RNA may be referred to herein as “stabilizing oligonucleotides.” In some examples, such oligonucleotides hybridize with an RNA and prevent or inhibit exonuclease mediated degradation. Inhibition of exonuclease mediated degradation includes, but is not limited to, reducing the extent of degradation of a particular RNA by exonucleases. For example, an exonuclease that processes only single stranded RNA may cleave a portion of the RNA up to a region where an oligonucleotide is hybridized with the RNA because the exonuclease cannot effectively process (e.g., pass through) the duplex region. Thus, in some embodiments, using an oligonucleotide that targets a particular region of an RNA makes it possible to control the extent of degradation of the RNA by exonucleases up to that region. For example, use of an oligonucleotide that hybridizes at an end of an RNA may reduce or eliminate degradation by an exonuclease that processes only single stranded RNAs from that end. For example, use of an oligonucleotide that hybridizes at the 5′ end of an RNA may reduce or eliminate degradation by an exonuclease that processes single stranded RNAs in a 5′ to 3′ direction. Similarly, use of an oligonucleotide that hybridizes at the 3′ end of an RNA may reduce or eliminate degradation by an exonuclease that processes single stranded RNAs in a 3′ to 5′ direction. In some embodiments, lower concentrations of an oligo may be used when the oligo hybridizes at both the 5′ and 3′ regions of the RNA. In some embodiments, an oligo that hybridizes at both the 5′ and 3′ regions of the RNA protects the 5′ and 3′ regions of the RNA from degradation (e.g., by an exonuclease). In some embodiments, an oligo that hybridizes at both the 5′ and 3′ regions of the RNA creates a pseudo-circular RNA (e.g., a circularized RNA with a region of the poly A tail that protrudes from the circle, see FIG. 3B). In some embodiments, a pseudo-circular RNA is translated at a higher efficiency than a non-pseudo-circular RNA.

In some embodiments, an oligonucleotide may be used that comprises multiple regions of complementarity with an RNA, such that at one region the oligonucleotide hybridizes at or near the 5′ end of the RNA and at another region it hybridizes at or near the 3′ end of the RNA, thereby preventing or inhibiting degradation of the RNA by exonucleases at both ends. In some embodiments, when an oligonucleotide hybridizes both at or near the 5′ end of an RNA and at or near the 3′ end of the RNA a circularized complex results that is protected from exonuclease mediated degradation. In some embodiments, when an oligonucleotide hybridizes both at or near the 5′ end of an mRNA and at or near the 3′ end of the mRNA, the circularized complex that results is protected from exonuclease mediated degradation and the mRNA in the complex retains its ability to be translated into a protein.

As used herein the term, “synthetic RNA” refers to a RNA produced through an in vitro transcription reaction or through artificial (non-natural) chemical synthesis. In some embodiments, a synthetic RNA is an RNA transcript. In some embodiments, a synthetic RNA encodes a protein. In some embodiments, the synthetic RNA is a functional RNA (e.g., a lncRNA, miRNA, etc.). In some embodiments, a synthetic RNA comprises one or more modified nucleotides. In some embodiments, a synthetic RNA is up to 0.5 kilobases (kb), 1 kb, 1.5 kb, 2 kb, 2.5 kb, 3 kb, 4 kb, 5 kb, 6 kb, 7 kb, 8 kb, 9 kb, 10 kb, 15 kb, 20 kb, 25 kb, 30 kb or more in length. In some embodiments, a synthetic RNA is in a range of 0.1 kb to 1 kb, 0.5 kb to 2 kb, 0.5 kb to 10 kb, 1 kb to 5 kb, 2 kb to 5 kb, 1 kb to 10 kb, 3 kb to 10 kb, 5 kb to 15 kb, or 1 kb to 30 kb in length.

As used herein, the term “RNA transcript” refers to an RNA that has been transcribed from a nucleic acid by a polymerase enzyme. An RNA transcript may be produced inside or outside of cells. For example, an RNA transcript may be produced from a DNA template encoding the RNA transcript using an in vitro transcription reaction that utilizes recombination or purified polymerase enzymes. An RNA transcript may also be produced from a DNA template (e.g., chromosomal gene, an expression vector) in a cell by an RNA polymerase (e.g., RNA polymerase I, II, or III). In some embodiments, the RNA transcript is a protein coding mRNA. In some embodiments, the RNA transcript is a non-coding RNA (e.g., a tRNA, rRNA, snoRNA, miRNA, ncRNA, long-noncoding RNA, shRNA). In some embodiments, RNA transcript is up to 0.5 kilobases (kb), 1 kb, 1.5 kb, 2 kb, 2.5 kb, 3 kb, 4 kb, 5 kb, 6 kb, 7 kb, 8 kb, 9 kb, 10 kb, 15 kb, 20 kb, 25 kb, 30 kb or more in length. In some embodiments, a RNA transcript is in a range of 0.1 kb to 1 kb, 0.5 kb to 2 kb, 0.5 kb to 10 kb, 1 kb to 5 kb, 2 kb to 5 kb, 1 kb to 10 kb, 3 kb to 10 kb, 5 kb to 15 kb, or 1 kb to 30 kb in length.

In some embodiments, the RNA transcript is capped post-transcriptionally, e.g., with a 7′-methylguanosine cap. In some embodiments, the 7′-methylguanosine is added to the RNA transcript by a guanylyltransferase during transcription (e.g., before the RNA transcript is 20-50 nucleotides long.) In some embodiments, the 7 ‘-methylguanosine is linked to the first transcribed nucleotide through a 5’-5′ triphosphate bridge. In some embodiments, the nucleotide immediately internal to the cap is an adenosine that is N6 methylated. In some embodiments, the first and second nucleotides immediately internal to the cap of the RNA transcript are not 2′-O-methylated. In some embodiments, the first nucleotide immediately internal to the cap of the RNA transcript is 2′-O-methylated. In some embodiments, the second nucleotide immediately internal to the cap of the RNA transcript is 2′-O-methylated. In some embodiments, the first and second nucleotides immediately internal to the cap of the RNA transcript are 2′-O-methylated.

In some embodiments, the RNA transcript is a non-capped transcript (e.g., a transcript produced from a mitochondrial gene). In some embodiments, the RNA transcript is a nuclear RNA that was capped but that has been decapped. In some embodiments, decapping of an RNA is catalyzed by the decapping complex, which may be composes of Dcp1 and Dcp2, e.g., that may compete with eIF-4E to bind the cap. In some embodiments, the process of RNA decapping involves hydrolysis of the 5′ cap structure on the RNA exposing a 5′ monophosphate. In some embodiments, this 5′ monophosphate is a substrate for the exonuclease XRN1. Accordingly, in some embodiments, an oligonucleotide that targets the 5′ region of an RNA may be used to stabilize (or restore stability) to a decapped RNA, e.g., protecting it from degradation by an exonuclease such as XRN1.

In some embodiments, in vitro transcription (e.g., performed via a T7 RNA polymerase or other suitable polymerase) may be used to produce an RNA transcript. In some embodiments transcription may be carried out in the presence of anti-reverse cap analog (ARCA) (TriLink Cat. # N-7003). In some embodiments, transcription with ARCA results in insertion of a cap (e.g., a cap analog (mCAP)) on the RNA in a desirable orientation.

In some embodiments, transcription is performed in the presence of one or more modified nucleotides (e.g., pseudouridine, 5-methylcytosine, etc.), such that the modified nucleotides are incorporated into the RNA transcript. It should be appreciated that any suitable modified nucleotide may be used, including, but not limited to, modified nucleotides that reduced immune stimulation, enhance translation and increase nuclease stability. Non-limiting examples of modified nucleotides that may be used include: 2′-amino-2′-deoxynucleotide, 2′-azido-2′-deoxynucleotide, 2′-fluoro-2′-deoxynucleotide, 2′-O-methyl-nucleotide, 2′ sugar super modifier, 2′-modified thermostability enhancer, 2′-fluoro-2′-deoxyadenosine-5′-triphosphate, 2′-fluoro-2′-deoxycytidine-5′-triphosphate, 2′-fluoro-2′-deoxyguanosine-5′-triphosphate, 2′-fluoro-2′-deoxyuridine-5′-triphosphate, 2′-O-methyladenosine-5′-triphosphate, 2′-O-methylcytidine-5′-triphosphate, 2′-O-methylguanosine-5′-triphosphate, 2′-O-methyluridine-5′-triphosphate, pseudouridine-5′-triphosphate, 2′-O-methylinosine-5′-triphosphate, 2′-amino-2′-deoxycytidine-5′-triphosphate, 2′-amino-2′-deoxyuridine-5′-triphosphate, 2′-azido-2′-deoxycytidine-5′-triphosphate, 2′-azido-2′-deoxyuridine-5′-triphosphate, 2′-O-methylpseudouridine-5′-triphosphate, 2′-O-methyl-5-methyluridine-5′-triphosphate, 2′-azido-2′-deoxyadenosine-5′-triphosphate, 2′-amino-2′-deoxyadenosine-5′-triphosphate, 2′-fluoro-thymidine-5′-triphosphate, 2′-azido-2′-deoxyguanosine-5′-triphosphate, 2′-amino-2′-deoxyguanosine-5′-triphosphate, and N4-methylcytidine-5′-triphosphate. In one embodiment, RNA degradation or processing can be reduced/prevented to elevate steady state RNA and, at least for protein-coding transcripts, protein levels. In some embodiments, a majority of degradation of RNA transcripts is done by exonucleases. In such embodiments, these enzymes start destroying RNA from either their 3′ or 5′ ends. By protecting the ends of the RNA transcripts from exonuclease enzyme activity, for instance, by hybridization of sequence-specific blocking oligonucleotides with proper chemistries for proper delivery, hybridization and stability within cells, RNA stability may be increase, along with protein levels for protein-coding transcripts.

In some embodiments, for the 5′ end, oligonucleotides may be used that are fully/partly complementary to 10-20 nts of the RNA 5′ end. In some embodiments, such oligonucleotides may have overhangs to form a hairpin (e.g., the 3′ nucleotide of the oligonucleotide can be, but not limited to, a C to interact with the mRNA 5′ cap's G nucleoside) to protect the RNA 5′ cap. In some embodiments, all nucleotides of an oligonucleotide may be complementary to the 5′ end of an RNA transcript, with or without few nucleotide overhangs to create a blunt or recessed 5′RNA-oligo duplex. In some embodiments, for the 3′ end, oligonucleotides may be partly complementary to the last several nucleotides of the RNA 3′ end, and optionally may have a poly(T)-stretch to protect the poly(A) tail from complete degradation (for transcripts with a poly(A)-tail). In some embodiments, similar strategies can be employed for other RNA species with different 5′ and 3′ sequence composition and structure (such as transcripts containing 3′ poly(U) stretches or transcripts with alternate 5′ structures). In some embodiments, oligonucleotides as described herein, including, for example, oligonucleotides with overhangs, may have higher specificity and sensitivity to their target RNA end regions compared to oligonucleotides designed to be perfectly complementary to RNA sequences, because the overhangs provide a destabilizing effect on mismatch regions and prefer binding in regions that are at the 5′ or 3′ ends of the RNAs. In some embodiments, oligonucleotides that protect the very 3′ end of the poly(A) tail with a looping mechanism (e.g., TTTTTTTTTTGGTTTTCC) (SEQ ID NO: 121). In some embodiments, this latter approach may nonspecifically target all protein-coding transcripts. However, in some embodiments, such oligonucleotides, may be useful in combination with other target-specific oligos.

In some embodiments, methods provided herein involve the use of an oligonucleotide that comprises a region of complementarity that is complementary with the RNA transcript at a position at or near the first transcribed nucleotide of the RNA transcript. In some embodiments, an oligonucleotide (e.g., an oligonucleotide that stabilizes an RNA transcript) comprises a region of complementarity that is complementary with the RNA transcript (e.g., with at least 5 contiguous nucleotides) at a position that begins within 100 nucleotides, within 50 nucleotides, within 30 nucleotides, within 20 nucleotides, within 10 nucleotides or within 5 nucleotides of the 5′-end of the transcript. In some embodiments, an oligonucleotide (e.g., an oligonucleotide that stabilizes an RNA transcript) comprises a region of complementarity that is complementary with the RNA transcript (e.g., with at least 5 contiguous nucleotides of the RNA transcript) at a position that begins at the 5′-end of the transcript. In some embodiments, an oligonucleotide (e.g., an oligonucleotide that stabilizes an RNA transcript) comprises a region of complementarity that is complementary with an RNA transcript at a position within a region of the 5′ untranslated region (5′ UTR) of the RNA transcript spanning from the transcript start site to 50, 100, 150, 200, 250, 500 or more nucleotides upstream from a translation start site (e.g., a start codon, AUG, arising in a Kozak sequence of the transcript).

In some embodiments, an RNA transcript is poly-adenylated. Polyadenylation refers to the post-transcriptional addition of a polyadenosine (poly(A)) tail to an RNA transcript. Both protein-coding and non-coding RNA transcripts may be polyadenylated. Poly(A) tails contain multiple adenosines linked together through internucleoside linkages. In some embodiments, a poly(A) tail may contain 10 to 50, 25 to 100, 50 to 200, 150 to 250 or more adenosines. In some embodiments, the process of polyadenlyation involves endonucleolytic cleavage of an RNA transcript at or near its 3′-end followed by one by one addition of multiple adenosines to the transcript by a polyadenylate polymerase, the first of which adenonsines is added to the transcript at the 3′ cleavage site. Thus, often a polyadenylated RNA transcript comprises transcribed nucleotides (and possibly edited nucleotides) linked together through internucleoside linkages that are linked at the 3′ end to a poly(A) tail. The location of the linkage between the transcribed nucleotides and poly(A) tail may be referred to herein as, a “polyadenylation junction.” In some embodiments, endonucleolytic cleavage may occur at any one of several possible sites in an RNA transcript. In such embodiments, the sites may be determined by sequence motifs in the RNA transcript that are recognized by endonuclease machinery, thereby guiding the position of cleavage by the machinery. Thus, in some embodiments, polyadenylation can produce different RNA transcripts from a single gene, e.g., RNA transcripts have different polyadenylation junctions. In some embodiments, length of a poly(A) tail may determine susceptibility of the RNA transcript to enzymatic degradation by exonucleases with 3′-5′ processing activity. In some embodiments, oligonucleotides that target an RNA transcript at or near its 3′ end target a region overlapping a polyadenylation junction. In some embodiments, such oligonucleotides may have at least 3, 4, 5, 6, 7, 8, 9, 10, 11, 12 or more nucleotides that are complementary with the transcribed portion of the transcript (5′ to the junction). In some embodiments, it is advantageous to have a limited number of nucleotides (e.g., T, U) complementary to the polyA side of the junction. In some embodiments, having a limited number of nucleotides complementary to the polyA side of the junction it is advantageous because it reduces toxicity associated with cross hybridization of the oligonucleotide to the polyadenylation region of non-target RNAs in cells. In some embodiments, the oligonucleotide has only 1, 2, 3, 4, 5, or 6 nucleotides complementary to the poly A region.

In some embodiments, methods provided herein involve the use of an oligonucleotide that hybridizes with a target RNA transcript at or near its 3′ end and prevents or inhibits degradation of the RNA transcript by 3′-5′ exonucleases. For example, in some embodiments, RNA stabilization methods provided herein involve the use of an oligonucleotide that comprises a region of complementarity that is complementary with the RNA transcript at a position within 100 nucleotides, within 50 nucleotides, within 30 nucleotides, within 20 nucleotides, within 10 nucleotides, within 5 nucleotides of the last transcribed nucleotide of the RNA transcript. In a case where the RNA transcript is a polyadenylated transcript, the last transcribed nucleotide of the RNA transcript is the first nucleotide upstream of the polyadenylation junction. In some embodiments, RNA stabilization methods provided herein involve the use of an oligonucleotide that comprises a region of complementarity that is complementary with the RNA transcript at a position immediately adjacent to or overlapping the polyadenylation junction of the RNA transcript. In some embodiments, RNA stabilization methods provided herein involve the use of an oligonucleotide that comprises a region of complementarity that is complementary with the RNA transcript within the poly(A) tail.

Methods for identifying transcript start sites and polyadenylation junctions are known in the art and may be used in selecting oligonucleotides that specifically bind to these regions for stabilizing RNA transcripts. In some embodiments, 3′ end oligonucleotides may be designed by identifying RNA 3′ ends using quantitative end analysis of poly-A tails. In some embodiments, 5′ end oligonucleotides may be designed by identifying 5′ start sites using Cap analysis gene expression (CAGE). Appropriate methods are disclosed, for example, in Ozsolak et al. Comprehensive Polyadenylation Site Maps in Yeast and Human Reveal Pervasive Alternative Polyadenylation. Cell. Volume 143, Issue 6, 2010, Pages 1018-1029; Shiraki, T, et al., Cap analysis gene expression for high-throughput analysis of transcriptional starting point and identification of promoter usage. Proc Natl Acad Sci USA. 100 (26): 15776-81.2003-12-23; and Zhao, X, et al., (2011). Systematic Clustering of Transcription Start Site Landscapes. PLoS ONE (Public Library of Science) 6 (8): e23409, the contents of each of which are incorporated herein by reference. Other appropriate methods for identifying transcript start sites and polyadenylation junctions may also be used, including, for example, RNA-Paired-end tags (PET) (See, e.g., Ruan X, Ruan Y. Methods Mol Biol. 2012; 809:535-62); use of standard EST databases; RACE combined with microarray or sequencing, PAS-Seq (See, e.g., Peter J. Shepard, et al., RNA. 2011 Apr.; 17(4): 761-772); and 3P-Seq (See, e.g., Calvin H. Jan, Nature. 2011 Jan. 6; 469(7328): 97-101; and others.

In some embodiments, an RNA transcript targeted by an oligonucleotide disclosed herein is an RNA transcript of a eukaryotic cell. In some embodiments, an RNA transcript targeted by an oligonucleotide disclosed herein is an RNA transcript of a cell of a vertebrate. In some embodiments, an RNA transcript targeted by an oligonucleotide disclosed herein is an RNA transcript of a cell of a mammal, e.g., a primate cell, mouse cell, rat cell, or human cell. In some embodiments, an RNA transcript targeted by an oligonucleotide disclosed herein is an RNA transcript of a cardiomyocyte. In some embodiments, an RNA transcript targeted by an oligonucleotide disclosed herein is an RNA transcribed in the nucleus of a cell. In some embodiments, an RNA transcript targeted by an oligonucleotide disclosed herein is an RNA transcribed in a mitochondrion of a cell. In some embodiments, an RNA transcript targeted by an oligonucleotide disclosed herein is an RNA transcript transcribed by a RNA polymerase II enzyme.

In some embodiments, an RNA transcript targeted by an oligonucleotide disclosed herein is an mRNA expressed from a gene selected from the group consisting of: FXN, THRB, HAMP, APOA1 and NR1H4. In some embodiments, the RNA transcript targeted by an oligonucleotide disclosed herein is an mRNA expressed from a gene selected from the group consisting of: THRB, HAMP, APOA1 and NR1H4. In some embodiments, an RNA transcript targeted by an oligonucleotide disclosed herein is an mRNA expressed from a gene selected from the group consisting of: THRB and NR1H4. RNA transcripts for these and other genes may be selected or identified experimentally, for example, using RNA sequencing (RNA-Seq) or other appropriate methods. RNA transcripts may also be selected based on information in public databases such as in UCSC, Ensembl and NCBI genome browsers and others. Non-limiting examples of RNA transcripts for certain genes are listed in Table 1.

TABLE 1
Non-limiting examples of RNA transcripts for certain genes

| GENE | | | |
|---|---|---|---|
| SYMBOL | MRNA | SPECIES | GENE NAME |
| APOA1 | NM_000039 | Homo sapiens | apolipoprotein A-I |
| APOA1 | NM_009692 | Mus musculus | apolipoprotein A-I |
| FXN | NM_001161706 | Homo sapiens | frataxin |
| FXN | NM_181425 | Homo sapiens | frataxin |
| FXN | NM_000144 | Homo sapiens | frataxin |
| FXN | NM_008044 | Mus musculus | frataxin |
| HAMP | NM_021175 | Homo sapiens | hepcidin antimicrobial |
| | | | peptide |
| HAMP | NM_032541 | Mus musculus | hepcidin antimicrobial |
| | | | peptide |
| THRB | NM_000461.4 | Homo sapiens | thyroid hormone receptor, |
| | | | beta |
| THRB | NM_001128176.2 | Homo sapiens | thyroid hormone receptor, |
| | | | beta |
| THRB | NM_001128177.1 | Homo sapiens | thyroid hormone receptor, |
| | | | beta |
| THRB | NM_001252634.1 | Homo sapiens | thyroid hormone receptor, |
| | | | beta |
| THRB | NM_001113417.1 | Mus musculus | thyroid hormone receptor, |
| | | | beta |
| THRB | NM_009380.3 | Mus musculus | thyroid hormone receptor, |
| | | | beta |
| NR1H4 | NM_001206977.1 | Homo sapiens | nuclear receptor subfamily 1, |
| | | | group H, member 4 |
| NR1H4 | NM_001206978.1 | Homo sapiens | nuclear receptor subfamily 1, |
| | | | group H, member 4 |
| NR1H4 | NM_001206979.1 | Homo sapiens | nuclear receptor subfamily 1, |
| | | | group H, member 4 |
| NR1H4 | NM_001206992.1 | Homo sapiens | nuclear receptor subfamily 1, |
| | | | group H, member 4 |
| NR1H4 | NM_001206993.1 | Homo sapiens | nuclear receptor subfamily 1, |
| | | | group H, member 4 |
| NR1H4 | NM_005123.3 | Homo sapiens | nuclear receptor subfamily 1, |
| | | | group H, member 4 |
| NR1H4 | NM_001163504.1 | Mus musculus | nuclear receptor subfamily 1, |
| | | | group H, member 4 |
| NR1H4 | NM_001163700.1 | Mus musculus | nuclear receptor subfamily 1, |
| | | | group H, member 4 |
| NR1H4 | NM_009108.2 | Mus musculus | nuclear receptor subfamily 1, |
| | | | group H, member 4 |
| PRKAA1 | NM_006251.5 | Homo sapiens | protein kinase, AMP- |
| | | | activated, alpha 1 catalytic |
| | | | subunit |
| PRKAA1 | NM_206907.3 | Homo sapiens | protein kinase, AMP- |
| | | | activated, alpha 1 catalytic |
| | | | subunit |
| PRKAA1 | NM_001013367.3 | Mus musculus | protein kinase, AMP- |
| | | | activated, alpha 1 catalytic |
| | | | subunit |
| PRKAA2 | NM_006252.3 | Homo sapiens | protein kinase, AMP- |
| | | | activated, alpha 2 catalytic |
| | | | subunit |
| PRKAA2 | NM_178143.2 | Mus musculus | protein kinase, AMP- |
| | | | activated, alpha 2 catalytic |
| | | | subunit |
| PRKAB1 | NM_006253.4 | Homo sapiens | protein kinase, AMP- |
| | | | activated, beta 1 non- |
| | | | catalytic subunit |
| PRKAB1 | NM_031869.2 | Mus musculus | protein kinase, AMP- |
| | | | activated, beta 1 non- |
| | | | catalytic subunit |
| PRKAB2 | NM_005399.4 | Homo sapiens | protein kinase, AMP- |
| | | | activated, beta 2 non- |
| | | | catalytic subunit |
| PRKAB2 | NM_182997.2 | Mus musculus | protein kinase, AMP- |
| | | | activated, beta 2 non- |
| | | | catalytic subunit |
| PRKAG1 | NM_001206709.1 | Homo sapiens | protein kinase, AMP- |
| | | | activated, gamma 1 non- |
| | | | catalytic subunit |
| PRKAG1 | NM_001206710.1 | Homo sapiens | protein kinase, AMP- |
| | | | activated, gamma 1 non- |
| | | | catalytic subunit |
| PRKAG1 | NM_002733.4 | Homo sapiens | protein kinase, AMP- |
| | | | activated, gamma 1 non- |
| | | | catalytic subunit |
| PRKAG1 | NM_016781.2 | Mus musculus | protein kinase, AMP- |
| | | | activated, gamma 1 non- |
| | | | catalytic subunit |
| PRKAG2 | NM_001040633.1 | Homo sapiens | protein kinase, AMP- |
| | | | activated, gamma 2 non- |
| | | | catalytic subunit |
| PRKAG2 | NM_001304527.1 | Homo sapiens | protein kinase, AMP- |
| | | | activated, gamma 2 non- |
| | | | catalytic subunit |
| PRKAG2 | NM_001304531.1 | Homo sapiens | protein kinase, AMP- |
| | | | activated, gamma 2 non- |
| | | | catalytic subunit |
| PRKAG2 | NM_016203.3 | Homo sapiens | protein kinase, AMP- |
| | | | activated, gamma 2 non- |
| | | | catalytic subunit |
| PRKAG2 | NM_024429.1 | Homo sapiens | protein kinase, AMP- |
| | | | activated, gamma 2 non- |
| | | | catalytic subunit |
| PRKAG2 | NM_001170555.1 | Mus musculus | protein kinase, AMP- |
| | | | activated, gamma 2 non- |
| | | | catalytic subunit |
| PRKAG2 | NM_001170556.1 | Mus musculus | protein kinase, AMP- |
| | | | activated, gamma 2 non- |
| | | | catalytic subunit |
| PRKAG2 | NM_145401.2 | Mus musculus | protein kinase, AMP- |
| | | | activated, gamma 2 non- |
| | | | catalytic subunit |
| PRKAG3 | NM_017431.2 | Homo sapiens | protein kinase, AMP- |
| | | | activated, gamma 3 non- |
| | | | catalytic subunit |
| PRKAG3 | NM_153744.3 | Mus musculus | protein kinase, AMP- |
| | | | activated, gamma 3 non- |
| | | | catalytic subunit |

### Oligonucleotides

Oligonucleotides provided herein are useful for stabilizing RNAs by inhibiting or preventing degradation of the RNAs (e.g., degradation mediated by exonucleases). Such oligonucleotides may be referred to as “stabilizing oligonucleotides”. In some embodiments, oligonucleotides hybridize at a 5′ and/or 3′ region of the RNA resulting in duplex regions that stabilize the RNA by preventing degradation by exonucleotides having single strand processing activity.

In some embodiments, oligonucleotides are provided having a region complementary with at least 5 consecutive nucleotides of a 5′ region of an RNA transcript. In some embodiments, oligonucleotides are provided having a region complementary with at least 5 consecutive nucleotides of a 3′-region of an RNA transcript. In some embodiments, oligonucleotides are provided having a first region complementary with at least 5 consecutive nucleotides of a 5′ region of an RNA transcript, and a second region complementary with at least 5 consecutive nucleotides of a 3′-region of an RNA transcript.

In some embodiments, oligonucleotides are provided having a region complementary with at least 5 consecutive nucleotides of the 5′-UTR of an mRNA transcript. In some embodiments, oligonucleotides are provided having a region complementary with at least 5 consecutive nucleotides of the 3′-UTR, poly(A) tail, or overlapping the polyadenylation junction of the mRNA transcript. In some embodiments, oligonucleotides are provided having a first region complementary with at least 5 consecutive nucleotides of the 5′-UTR of an mRNA transcript, and a second region complementary with at least 5 consecutive nucleotides of the 3′-UTR, poly(A) tail, or overlapping the polyadenylation junction of the mRNA transcript.

In some embodiments, oligonucleotides are provided that have a region of complementarity that is complementary to an RNA transcript in proximity to the 5′-end of the RNA transcript. In such embodiments, the nucleotide at the 3′-end of the region of complementarity of the oligonucleotides may be complementary with the RNA transcript at a position that is within 10 nucleotides, within 20 nucleotides, within 30 nucleotides, within 40 nucleotides, within 50 nucleotides, or within 100 nucleotides, within 200 nucleotides, within 300 nucleotides, within 400 nucleotides or more of the transcription start site of the RNA transcript.

In some embodiments, oligonucleotides are provided that have a region of complementarity that is complementary to an RNA transcript in proximity to the 3′-end of the RNA transcript. In such embodiments, the nucleotide at the 3′-end and/or 5′ end of the region of complementarity may be complementary with the RNA transcript at a position that is within 10 nucleotides, within 20 nucleotides, within 30 nucleotides, within 40 nucleotides, within 50 nucleotides, within 100 nucleotides, within 200 nucleotides, within 300 nucleotides, within 400 nucleotides or more of the 3′-end of the RNA transcript. In some embodiments, if the target RNA transcript is polyadenylated, the nucleotide at the 3′-end of the region of complementarity of the oligonucleotide may be complementary with the RNA transcript at a position that is within 10 nucleotides, within 20 nucleotides, within 30 nucleotides, within 40 nucleotides, within 50 nucleotides, within 100 nucleotides, within 200 nucleotides, within 300 nucleotides, within 400 nucleotides or more of polyadenylation junction. In some embodiments, an oligonucleotide that targets a 3′ region of an RNA comprises a region of complementarity that is a stretch of pyrimidines (e.g., 4 to 10 or 5 to 15 thymine nucleotides) complementary with adenines.

In some embodiments, combinations of 5′ targeting and 3′ targeting oligonucleotides are contacted with a target RNA. In some embodiments, the 5′ targeting and 3′ targeting oligonucleotides a linked together via a linker (e.g., a stretch of nucleotides non-complementary with the target RNA). In some embodiments, the region of complementarity of the 5′ targeting oligonucleotide is complementary to a region in the target RNA that is at least 2, 5, 10, 20, 50, 100, 500, 1000, 5000, 10000 nucleotides upstream from the region of the target RNA that is complementary to the region of complementarity of the 3′ end targeting oligonucleotide.

In some embodiments, oligonucleotides are provided that have the general formula 5′-X₁-X₂-3′, in which X₁ has a region of complementarity that is complementary with an RNA transcript (e.g., with at least 5 contiguous nucleotides of the RNA transcript). In some embodiments, the nucleotide at the 3′-end of the region of complementary of X₁ may be complementary with a nucleotide in proximity to the transcription start site of the RNA transcript. In some embodiments, the nucleotide at the 3′-end of the region of complementary of X₁ may be complementary with a nucleotide that is present within 1, 2, 3, 4, 5, 6, 7, 8, 9, or 10 nucleotides of the transcription start site of the RNA transcript. In some embodiments, the nucleotide at the 3′-end of the region of complementary of X₁ may be complementary with the nucleotide at the transcription start site of the RNA transcript.

In some embodiments, X₁ comprises 5 to 10 nucleotides, 5 to 15 nucleotides, 5 to 25 nucleotides, 10 to 25 nucleotides, 5 to 20 nucleotides, or 15 to 30 nucleotides. In some embodiments, X₁ comprises 5, 6, 7, 8, 9, 10, 11, 12, 13, 14, 15, 16, 17, 18, 19, 20, 25, 30 or more nucleotides. In some embodiments, the region of complementarity of X₁ may be complementary with at least 4, at least 5, at least 6, at least 7, at least 8, at least 9, or at least 10 contiguous nucleotides of the RNA transcript. In some embodiments, the region of complementarity of X₁ may be complementary with 4, 5, 6, 7, 8, 9, 10, 11, 12, 13, 14, 15, 16, 17, 18, 19, 20 or more contiguous nucleotides of the RNA transcript.

In some embodiments, X₂ is absent. In some embodiments, X₂ comprises 1 to 10, 1 to 20 nucleotides, 1 to 25 nucleotides, 5 to 20 nucleotides, 5 to 30 nucleotides, 5 to 40 nucleotides, or 5 to 50 nucleotides. In some embodiments, X₂ comprises 5, 6, 7, 8, 9, 10, 11, 12, 13, 14, 15, 16, 17, 18, 19, 20, 25, 30, 35, 40, 45, 50 or more nucleotides. In some embodiments, X₂ comprises a region of complementarity complementary with at least 4, at least 5, at least 6, at least 7, at least 8, at least 9, or at least 10 contiguous nucleotides of the RNA transcript. In some embodiments, X₂ comprises a region of complementarity complementary with 4, 5, 6, 7, 8, 9, 10, 11, 12, 13, 14, 15, 16, 17, 18, 19, 20 or more contiguous nucleotides of the RNA transcript.

In some embodiments, the RNA transcript has a 7-methylguanosine cap at its 5′-end. In some embodiments, the nucleotide at the 3′-end of the region of complementary of X₁ is complementary with the nucleotide of the RNA transcript that is immediately internal to the 7-methylguanosine cap or in proximity to the cap (e.g., with 10 nucleotides of the cap). In some embodiments, at least the first nucleotide at the 5′-end of X₂ is a pyrimidine complementary with guanine (e.g., a cytosine or analogue thereof). In some embodiments, the first and second nucleotides at the 5′-end of X₂ are pyrimidines complementary with guanine. Thus, in some embodiments, at least one nucleotide at the 5′-end of X₂ is a pyrimidine that may form stabilizing hydrogen bonds with the 7-methylguanosine of the cap.

In some embodiments, X₂ forms a stem-loop structure. In some embodiments, X₂ comprises the formula 5′-Y₁-Y₂-Y₃-3′, in which X₂ forms a stem-loop structure having a loop region comprising the nucleotides of Y₂ and a stem region comprising at least two contiguous nucleotides of Y₁ hybridized with at least two contiguous nucleotides of Y₃. In some embodiments, the stem region comprises 1-6, 1-5, 2-5, 1-4, 2-4 or 2-3 nucleotides. In some embodiments, the stem region comprises LNA nucleotides. In some embodiments, the stem region comprises 1-6, 1-5, 2-5, 1-4, 2-4 or 2-3 LNA nucleotides. In some embodiments, Y₁ and Y₃ independently comprise 2 to 10 nucleotides, 2 to 20 nucleotides, 2 to 25 nucleotides, or 5 to 20 nucleotides. In some embodiments, Y₁ and Y₃ independently comprise 2, 3, 4, 5, 6, 7, 8, 9, 10, 11, 12, 13, 14, 15, 16, 17, 18, 19, 20, 25 or more nucleotides. In some embodiments, Y₂ comprises 3 to 10 nucleotides, 3 to 15 nucleotides, 3 to 25 nucleotides, or 5 to 20 nucleotides. In some embodiments, Y₂ comprises 3, 4, 5, 6, 7, 8, 9, 10, 11, 12, 13, 14, 15, 16, 17, 18, 19, 20, 25 or more nucleotides. In some embodiments, Y₂ comprises 2-8, 2-7, 2-6, 2-5, 3-8, 3-7, 3-6, 3-5 or 3-4 nucleotides. In some embodiments, Y₂ comprises at least one DNA nucleotide. In some embodiments, the nucleotides of Y₂ comprise at least one (e.g., 1, 2, 3, 4, 5, 6, 7, 8, 9, 10, 11, 12, 13, 14, 15, 16, 17, 18, 19, 20, 25 or more adenines). In some embodiments, Y₃ comprises 1-5, 1-4, 1-3 or 1-2 nucleotides following the 3′ end of the stem region. In some embodiments, the nucleotides of Y₃ following the 3′ end of the stem region are DNA nucleotides. In some embodiments, Y₃ comprises a pyrimidine complementary with guanine (e.g., cytosine or an analogue thereof). In some embodiments, Y₃ comprises one or more (e.g., two) pyrimidines complementary with guanine at a position following the 3′-end of the stem region (e.g., 1, 2, 3 or more nucleotide after the 3′-end of the stem region). Thus, in embodiments where the RNA transcript is capped, Y₃ may have a pyrimidine that forms stabilizing hydrogen bonds with the 7-methylguanosine of the cap.

In some embodiments, X₁ and X₂ are complementary with non-overlapping regions of the RNA transcript. In some embodiments, X₁ comprises a region complementary with a 5′ region of the RNA transcript and X₂ comprises a region complementary with a 3′ region of the RNA transcript. For example, if the RNA transcript is polyadenylated, X₂ may comprise a region of complementarity that is complementary with the RNA transcript at a region within 100 nucleotides, within 50 nucleotides, within 25 nucleotides or within 10 nucleotides of the polyadenylation junction of the RNA transcript. In some embodiments, X₂ comprises a region of complementarity that is complementary with the RNA transcript immediately adjacent to or overlapping the polyadenylation junction of the RNA transcript. In some embodiments, X₂ comprises at least 2 consecutive pyrimidine nucleotides (e.g., 5 to 15 pyrimidine nucleotides) complementary with adenine nucleotides of the poly(A) tail of the RNA transcript.

In some embodiments, oligonucleotides are provided that comprise the general formula 5′-X₁-X₂-3′, in which X₁ comprises at least 2 nucleotides that form base pairs with adenine (e.g., thymidines or uridines or analogues thereof); and X₂ comprises a region of complementarity that is complementary with at least 3 contiguous nucleotides of a poly-adenylated RNA transcript, wherein the nucleotide at the 5′-end of the region of complementary of X₂ is complementary with the nucleotide of the RNA transcript that is immediately internal to the poly-adenylation junction of the RNA transcript. In such embodiments, X₁ may comprises 2 to 10, 2 to 20, 5 to 15 or 5 to 25 nucleotides and X₂ may independently comprises 2 to 10, 2 to 20, 5 to 15 or 5 to 25 nucleotides.

In some embodiments, compositions are provided that comprise a first oligonucleotide comprising at least 5 nucleotides (e.g., of 5 to 25 nucleotides) linked through internucleoside linkages, and a second oligonucleotide comprising at least 5 nucleotides (e.g., of 5 to 25 nucleotides) linked through internucleoside linkages, in which the the first oligonucleotide is complementary with at least 5 consecutive nucleotides in proximity to the 5′-end of an RNA transcript and the second oligonucleotide is complementary with at least 5 consecutive nucleotides in proximity to the 3′-end of an RNA transcript. In some embodiments, the 5′ end of the first oligonucleotide is linked with the 3′ end of the second oligonucleotide. In some embodiments, the 3′ end of the first oligonucleotide is linked with the 5′ end of the second oligonucleotide. In some embodiments, the 5′ end of the first oligonucleotide is linked with the 5′ end of the second oligonucleotide. In some embodiments, the 3′ end of the first oligonucleotide is linked with the 3′ end of the second oligonucleotide.

In some embodiments, the first oligonucleotide and second oligonucleotide are joined by a linker. The term “linker” generally refers to a chemical moiety that is capable of covalently linking two or more oligonucleotides. In some embodiments, a linker is resistant to cleavage in certain biological contexts, such as in a mammalian cell extract, such as an endosomal extract. However, in some embodiments, at least one bond comprised or contained within the linker is capable of being cleaved (e.g., in a biological context, such as in a mammalian extract, such as an endosomal extract), such that at least two oligonucleotides are no longer covalently linked to one another after bond cleavage. In some embodiments, the linker is not an oligonucleotide having a sequence complementary with the RNA transcript. In some embodiments, the linker is an oligonucleotide (e.g., 2-8 thymines). In some embodiments, the linker is a polypeptide. Other appropriate linkers may also be used, including, for example, linkers disclosed in International Patent Application Publication WO 2013/040429 A1, published on Mar. 21, 2013, and entitled MULTIMERIC ANTISENSE OLIGONUCLEOTIDES. The contents of this publication relating to linkers are incorporated herein by reference in their entirety.

An oligonucleotide may have a region of complementarity with a target RNA transcript (e.g., a mammalian mRNA transcript) that has less than a threshold level of complementarity with every sequence of nucleotides, of equivalent length, of an off-target RNA transcript. For example, an oligonucleotide may be designed to ensure that it does not have a sequence that targets RNA transcripts in a cell other than the target RNA transcript. The threshold level of sequence identity may be 50%, 60%, 70%, 80%, 85%, 90%, 95%, 99% or 100% sequence identity.

An oligonucleotide may be complementary to RNA transcripts encoded by homologues of a gene across different species (e.g., a mouse, rat, rabbit, goat, monkey, etc.) In some embodiments, oligonucleotides having these characteristics may be tested in vivo or in vitro for efficacy in multiple species (e.g., human and mouse). This approach also facilitates development of clinical candidates for treating human disease by selecting a species in which an appropriate animal exists for the disease.

In some embodiments, the region of complementarity of an oligonucleotide is complementary with at least 8 to 15, 8 to 30, 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of a target RNA. In some embodiments, the region of complementarity is complementary with at least 8 consecutive nucleotides of a target RNA.

Complementary, as the term is used in the art, refers to the capacity for precise pairing between two nucleotides. For example, if a nucleotide at a certain position of an oligonucleotide is capable of hydrogen bonding with a nucleotide at a corresponding position of a target RNA, then the nucleotide of the oligonucleotide and the nucleotide of the target RNA are complementary to each other at that position. The oligonucleotide and target RNA are complementary to each other when a sufficient number of corresponding positions in each molecule are occupied by nucleotides that can hydrogen bond with each other through their bases. Thus, “complementary” is a term which is used to indicate a sufficient degree of complementarity or precise pairing such that stable and specific binding occurs between the oligonucleotide and target RNA. For example, if a base at one position of an oligonucleotide is capable of hydrogen bonding with a base at the corresponding position of a target RNA, then the bases are considered to be complementary to each other at that position. 100% complementarity is not required.

An oligonucleotide may be at least 80% complementary to (optionally one of at least 85%, 90%, 91%, 92%, 93%, 94%, 95%, 96%, 97%, 98%, 99% or 100% complementary to) the consecutive nucleotides of a target RNA. In some embodiments an oligonucleotide may contain 1, 2 or 3 base mismatches compared to the portion of the consecutive nucleotides of the target RNA. In some embodiments an oligonucleotide may have up to 3 mismatches over 15 bases, or up to 2 mismatches over 10 bases.

In some embodiments, a complementary nucleic acid sequence need not be 100% complementary to that of its target to be specifically hybridizable. In some embodiments, an oligonucleotide for purposes of the present disclosure is specifically hybridizable with a target RNA when hybridization of the oligonucleotide to the target RNA prevents or inhibits degradation of the target RNA, and when there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80 or more nucleotides in length. In some embodiments, the oligonucleotide is 8 to 50, 10 to 30, 9 to 20, 15 to 30 or 8 to 80 nucleotides in length.

Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

In some embodiments, any one or more thymidine (T) nucleotides (or modified nucleotide thereof) or uridine (U) nucleotides (or a modified nucleotide thereof) in a sequence provided herein, including a sequence provided in the sequence listing, may be replaced with any other nucleotide suitable for base pairing (e.g., via a Watson-Crick base pair) with an adenosine nucleotide. In some embodiments, any one or more thymidine (T) nucleotides (or modified nucleotide thereof) or uridine (U) nucleotides (or a modified nucleotide thereof) in a sequence provided herein, including a sequence provided in the sequence listing, may be suitably replaced with a different pyrimidine nucleotide or vice versa. In some embodiments, any one or more thymidine (T) nucleotides (or modified nucleotide thereof) in a sequence provided herein, including a sequence provided in the sequence listing, may be suitably replaced with a uridine (U) nucleotide (or a modified nucleotide thereof) or vice versa.

In some embodiments, an oligonucleotide may have a sequence that does not contain guanosine nucleotide stretches (e.g., 3 or more, 4 or more, 5 or more, 6 or more consecutive guanosine nucleotides). In some embodiments, oligonucleotides having guanosine nucleotide stretches have increased non-specific binding and/or off-target effects, compared with oligonucleotides that do not have guanosine nucleotide stretches. Contiguous runs of three or more Gs or Cs may not be preferable in some embodiments. Accordingly, in some embodiments, the oligonucleotide does not comprise a stretch of three or more guanosine nucleotides.

An oligonucleotide may have a sequence that is has greater than 30% G-C content, greater than 40% G-C content, greater than 50% G-C content, greater than 60% G-C content, greater than 70% G-C content, or greater than 80% G-C content. An oligonucleotide may have a sequence that has up to 100% G-C content, up to 95% G-C content, up to 90% G-C content, or up to 80% G-C content. In some embodiments, GC content of an oligonucleotide is preferably between about 30-60%.

It is to be understood that any oligonucleotide provided herein can be excluded.

In some embodiments, it has been found that oligonucleotides disclosed herein may increase stability of a target RNA by at least about 50% (i.e. 150% of normal or 1.5 fold), or by about 2 fold to about 5 fold. In some embodiments, stability (e.g., stability in a cell) may be increased by at least about 15 fold, 20 fold, 30 fold, 40 fold, 50 fold or 100 fold, or any range between any of the foregoing numbers. In some embodiments, increased mRNA stability has been shown to correlate to increased protein expression. Similarly, in some embodiments, increased stability of non-coding positively correlates with increased activity of the RNA.

It is understood that any reference to uses of oligonucleotides or other molecules throughout the description contemplates use of the oligonucleotides or other molecules in preparation of a pharmaceutical composition or medicament for use in the treatment of condition or a disease associated with decreased levels or activity of a RNA transcript. Thus, as one nonlimiting example, this aspect of the invention includes use of oligonucleotides or other molecules in the preparation of a medicament for use in the treatment of disease, wherein the treatment involves posttranscriptionally altering protein and/or RNA levels in a targeted manner.

### Oligonucleotide Modifications

In some embodiments, oligonucleotides are provided with chemistries suitable for delivery, hybridization and stability within cells to target and stabilize RNA transcripts. Furthermore, in some embodiments, oligonucleotide chemistries are provided that are useful for controlling the pharmacokinetics, biodistribution, bioavailability and/or efficacy of the oligonucleotides. Accordingly, oligonucleotides described herein may be modified, e.g., comprise a modified sugar moiety, a modified internucleoside linkage, a modified nucleotide and/or combinations thereof. In addition, the oligonucleotides may exhibit one or more of the following properties: do not induce substantial cleavage or degradation of the target RNA; do not cause substantially complete cleavage or degradation of the target RNA; do not activate the RNAse H pathway; do not activate RISC; do not recruit any Argonaute family protein; are not cleaved by Dicer; do not mediate alternative splicing; are not immune stimulatory; are nuclease resistant; have improved cell uptake compared to unmodified oligonucleotides; are not toxic to cells or mammals; and may have improved endosomal exit.

Oligonucleotides that are designed to interact with RNA to modulate gene expression are a distinct subset of base sequences from those that are designed to bind a DNA target (e.g., are complementary to the underlying genomic DNA sequence from which the RNA is transcribed).

Any of the oligonucleotides disclosed herein may be linked to one or more other oligonucleotides disclosed herein by a linker, e.g., a cleavable linker.

Oligonucleotides of the invention can be stabilized against nucleolytic degradation such as by the incorporation of a modification, e.g., a nucleotide modification. For example, nucleic acid sequences of the invention include a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (T-O-M0E), 2?-O-aminopropyl (2?-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-0 atom and the 4′-C atom.

Any of the modified chemistries or formats of oligonucleotides described herein can be combined with each other, and that one, two, three, four, five, or more different types of modifications can be included within the same molecule.

In some embodiments, the oligonucleotide may comprise at least one ribonucleotide, at least one deoxyribonucleotide, and/or at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein and known in the art. In some embodiments, the oligonucleotide comprises a nucleotide analog disclosed in one of the following United States Patent or Patent Application Publications: U.S. Pat. No. 7,399,845, U.S. Pat. No. 7,741,457, U.S. Pat. No. 8,022,193, U.S. Pat. No. 7,569,686, U.S. Pat. No. 7,335,765, U.S. Pat. No. 7,314,923, U.S. Pat. No. 7,335,765, and U.S. Pat. No. 7,816,333, US 20110009471, the entire contents of each of which are incorporated herein by reference for all purposes. The oligonucleotide may have one or more 2′ O-methyl nucleotides. The oligonucleotide may consist entirely of 2′ O-methyl nucleotides.

Often an oligonucleotide has one or more nucleotide analogues. For example, an oligonucleotide may have at least one nucleotide analogue that results in an increase in Tₘ of the oligonucleotide in a range of 1° C., 2° C., 3° C., 4° C., or 5° C. compared with an oligonucleotide that does not have the at least one nucleotide analogue. An oligonucleotide may have a plurality of nucleotide analogues that results in a total increase in Tₘ of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the nucleotide analogue.

The oligonucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are nucleotide analogues. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are nucleotide analogues.

The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are nucleotide analogues. Optionally, the oligonucleotides may have every nucleotide except 1, 2, 3, 4, 5, 6, 7, 8, 9, or 10 nucleotides modified.

The oligonucleotide may consist entirely of bridged nucleotides (e.g., LNA nucleotides, cEt nucleotides, ENA nucleotides). The oligonucleotide may comprise alternating deoxyribonucleotides and 2′-fluoro-deoxyribonucleotides. The oligonucleotide may comprise alternating deoxyribonucleotides and 2′-O-methyl nucleotides. The oligonucleotide may comprise alternating deoxyribonucleotides and ENA nucleotide analogues. The oligonucleotide may comprise alternating deoxyribonucleotides and LNA nucleotides. The oligonucleotide may comprise alternating LNA nucleotides and 2′-O-methyl nucleotides. The oligonucleotide may have a 5′ nucleotide that is a bridged nucleotide (e.g., a LNA nucleotide, cEt nucleotide, ENA nucleotide). The oligonucleotide may have a 5′ nucleotide that is a deoxyribonucleotide.

The oligonucleotide may comprise deoxyribonucleotides flanked by at least one bridged nucleotide (e.g., a LNA nucleotide, cEt nucleotide, ENA nucleotide) on each of the 5′ and 3′ ends of the deoxyribonucleotides. The oligonucleotide may comprise deoxyribonucleotides flanked by 1, 2, 3, 4, 5, 6, 7, 8 or more bridged nucleotides (e.g., LNA nucleotides, cEt nucleotides, ENA nucleotides) on each of the 5′ and 3′ ends of the deoxyribonucleotides. The 3′ position of the oligonucleotide may have a 3′ hydroxyl group. The 3′ position of the oligonucleotide may have a 3′ thiophosphate.

The oligonucleotide may be conjugated with a label. For example, the oligonucleotide may be conjugated with a biotin moiety, cholesterol, Vitamin A, folate, sigma receptor ligands, aptamers, peptides, such as CPP, hydrophobic molecules, such as lipids, ligands of the asialoglycoprotein receptor (ASGPR), such as GalNac, or dynamic polyconjugates and variants thereof at its 5′ or 3′ end.

Preferably an oligonucleotide comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the oligonucleotides are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric oligonucleotides of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, an oligonucleotide comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, oligonucleotides may have phosphorothioate backbones; heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and Min et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other preferred modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties). Preferred ENAs include, but are not limited to, 2′-0,4′-C-ethylene-bridged nucleic acids.

Examples of LNAs are described in WO/2008/043753 and include compounds of the following general formula.

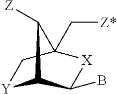

where X and Y are independently selected among the groups —O—,

—S—, —N(H)—, N(R)—, —CH₂— or —CH— (if part of a double bond),

—CH₂—O—, —CH₂—S—, —CH₂—N(H)—, —CH₂—N(R)—, —CH₂—CH₂— or —CH₂—CH— (if part of a double bond),

—CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

Preferably, the LNA used in the oligonucleotides described herein comprises at least one LNA unit according any of the formulas

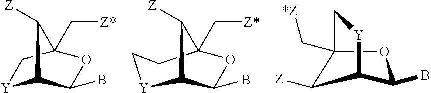

wherein Y is —O—, —S—, —NH—, or N(R^{H}); Z and Z* are independently selected among an internucleoside linkage, a terminal group or a protecting group; B constitutes a natural or non-natural nucleotide base moiety, and RH is selected from hydrogen and C₁₋₄-alkyl.

In some embodiments, the Locked Nucleic Acid (LNA) used in the oligonucleotides described herein comprises at least one Locked Nucleic Acid (LNA) unit according any of the formulas shown in Scheme 2 of PCT/DK2006/000512.

In some embodiments, the LNA used in the oligomer of the invention comprises internucleoside linkages selected from -0-P(O)₂—O—, —O—P(O,S)—O—, -0-P(S)₂—O—, —S—P(O)₂—O—, —S—P(O,S)—O—, —S—P(S)₂—O—, -0-P(O)₂—S—, —O—P(O,S)—S—, —S—P(O)₂—S—, —O—PO(R^{H})—O—, O—PO(OCH₃)—O—, —O—PO(NR^{H})—O—, -0-PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl.

Other examples of LNA units are shown below:

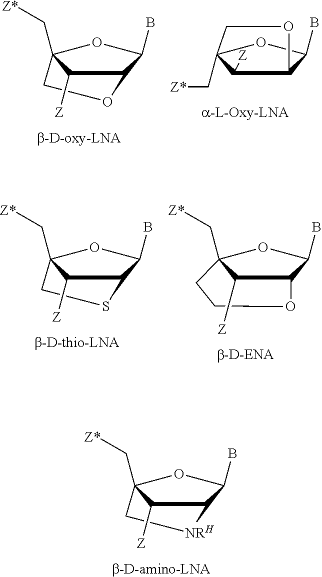

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

LNAs are described in additional detail herein.

One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂ CH₃; ONO₂; NO₂; N₃; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al, HeIv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-′7′7; and Gebeyehu, G., et al. Nucl. Acids Res., 15:4513 (1987)). A “universal” base known in the art, e.g., inosine, can also be included. 5-Me-C substitutions have been shown to increase nucleic acid duplex stability by 0.6-1.2° C. (Sanghvi, in Crooke, and Lebleu, eds., Antisense Research and Applications, CRC Press, Boca Raton, 1993, pp. 276-278) and may be used as base substitutions.

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Oligonucleotides can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine. In some embodiments, a cytosine is substituted with a 5-methylcytosine. In some embodiments, an oligonucleotide has 2, 3, 4, 5, 6, 7, or more cytosines substituted with a 5-methylcytosines. In some embodiments, an oligonucleotide does not have 2, 3, 4, 5, 6, 7, or more consecutive 5-methylcytosines. In some embodiments, an LNA cytosine nucleotide is replaced with an LNA 5-methylcytosine nucleotide.

Further, nucleobases comprise those disclosed in U.S. Pat. No. 3,687,808, those disclosed in “The Concise Encyclopedia of Polymer Science And Engineering”, pages 858-859, Kroschwitz, ed. John Wiley & Sons, 1990, those disclosed by Englisch et al., Angewandle Chemie, International Edition, 1991, 30, page 613, and those disclosed by Sanghvi, Chapter 15, Antisense Research and Applications,” pages 289-302, Crooke, and Lebleu, eds., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, et al., eds, “Antisense Research and Applications,” CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the oligonucleotides are chemically linked to one or more moieties or conjugates that enhance the activity, cellular distribution, or cellular uptake of the oligonucleotide. For example, one or more oligonucleotides, of the same or different types, can be conjugated to each other; or oligonucleotides can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In some embodiments, oligonucleotide modification include modification of the 5′ or 3′ end of the oligonucleotide. In some embodiments, the 3′ end of the oligonucleotide comprises a hydroxyl group or a thiophosphate. It should be appreciated that additional molecules (e.g. a biotin moiety or a fluorophor) can be conjugated to the 5′ or 3′ end of an oligonucleotide. In some embodiments, an oligonucleotide comprises a biotin moiety conjugated to the 5′ nucleotide.

In some embodiments, an oligonucleotide comprises locked nucleic acids (LNA), ENA modified nucleotides, 2′-O-methyl nucleotides, or 2′-fluoro-deoxyribonucleotides. In some embodiments, an oligonucleotide comprises alternating deoxyribonucleotides and 2′-fluoro-deoxyribonucleotides. In some embodiments, an oligonucleotide comprises alternating deoxyribonucleotides and 2′-O-methyl nucleotides. In some embodiments, an oligonucleotide comprises alternating deoxyribonucleotides and ENA modified nucleotides. In some embodiments, an oligonucleotide comprises alternating deoxyribonucleotides and locked nucleic acid nucleotides. In some embodiments, an oligonucleotide comprises alternating locked nucleic acid nucleotides and 2′-O-methyl nucleotides.

In some embodiments, the 5′ nucleotide of the oligonucleotide is a deoxyribonucleotide. In some embodiments, the 5′ nucleotide of the oligonucleotide is a locked nucleic acid nucleotide. In some embodiments, the nucleotides of the oligonucleotide comprise deoxyribonucleotides flanked by at least one locked nucleic acid nucleotide on each of the 5′ and 3′ ends of the deoxyribonucleotides. In some embodiments, the nucleotide at the 3′ position of the oligonucleotide has a 3′ hydroxyl group or a 3′ thiophosphate.

In some embodiments, an oligonucleotide comprises phosphorothioate internucleotide linkages. In some embodiments, an oligonucleotide comprises phosphorothioate internucleotide linkages between at least two nucleotides. In some embodiments, an oligonucleotide comprises phosphorothioate internucleotide linkages between all nucleotides.

It should be appreciated that an oligonucleotide can have any combination of modifications as described herein.

The oligonucleotide may comprise a nucleotide sequence having one or more of the following modification patterns.

(a) (X)Xxxxxx, (X)xXxxxx, (X)xxXxxx, (X)xxxXxx, (X)xxxxXx and (X)xxxxxX,

(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,

(c) (X)XXXxxx, (X)xXXXxx, (X)xxXXXx, (X)xxxXXX, (X)XXxXxx, (X)XXxxXx,

(X)XXxxxX, (X)xXXxXx, (X)xXXxxX, (X)xxXXxX, (X)XxXXxx, (X)XxxXXx (X)XxxxXX, (X)xXxXXx, (X)xXxxXX, (X)xxXxXX, (X)xXxXxX and (X)XxXxXx,

(d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXxXXx, (X)XXXxxX, (X)XXXxXx, and (X)XXXXxx,

(e) (X)xXXXXX, (X)XxXXXX, (X)XXxXXX, (X)XXXxXX, (X)XXXXxX and (X)XXXXXx, and

(f) XXXXXX, XxXXXXX, XXxXXXX, XXXxXXX, XXXXxXX, XXXXXxX and XXXXXXx, in which “X” denotes a nucleotide analogue, (X) denotes an optional nucleotide analogue, and “x” denotes a DNA or RNA nucleotide unit. Each of the above listed patterns may appear one or more times within an oligonucleotide, alone or in combination with any of the other disclosed modification patterns.

### Methods for Modulating Gene Expression

In one aspect, the invention relates to methods for modulating (e.g., increasing) stability of RNA transcripts in cells (e.g., human liver cells). The cells can be in vitro, ex vivo, or in vivo (e.g., in a human subject). The cells can be in a subject (e.g. a human subject) who has a disease or condition resulting from reduced expression or activity of the RNA transcript (e.g., an RNA transcript expressed in a human liver cell) or its corresponding protein product in the case of mRNAs. In some embodiments, methods for modulating stability of RNA transcripts in cells (e.g., human liver cells) comprise delivering to the cell an oligonucleotide that targets the RNA and prevents or inhibits its degradation by exonucleases. In some embodiments, delivery of an oligonucleotide to the cell results in an increase in stability of a target RNA that is at least 5%, 10%, 20%, 30%, 40%, 50%, 60%, 70%, 80%, 90%, 100%, 200% or more greater than a level of stability of the target RNA in a control cell. An appropriate control cell may be a cell to which an oligonucleotide has not been delivered or to which a negative control has been delivered (e.g., a scrambled oligo, a carrier, etc.).

Any human liver cell is contemplated herein. Exemplary liver cells include hepatocytes, sinusoidal endothelial cells, Kupffer cells, and stellate cells. In some embodiments, the human liver cell is a human hepatocyte.

Another aspect of the invention provides methods of treating a disease or condition associated with low levels of a particular RNA in a subject (e.g., a human subject). Accordingly, in some embodiments, methods are provided that comprise administering to a subject (e.g. a human) a composition comprising an oligonucleotide as described herein to increase mRNA stability in cells of the subject for purposes of increasing protein levels (e.g., in the liver of the human subject). In some embodiments, the increase in protein levels is at least 5%, 10%, 20%, 30%, 40%, 50%, 60%, 70%, 80%, 90%, 100%, 200%, or more, higher than the amount of a protein in the subject (e.g., in a cell or tissue of the subject) before administering or in a control subject which has not been administered the oligonucleotide or that has been administered a negative control (e.g., a scrambled oligo, a carrier, etc.). In some embodiments, methods are provided that comprise administering to a subject (e.g. a human) a composition comprising an oligonucleotide as described herein to increase stability of non-coding RNAs in cells of the subject for purposes of increasing activity of those non-coding RNAs.

A subject can include a non-human mammal, e.g. mouse, rat, guinea pig, rabbit, cat, dog, goat, cow, horse, or non-human primate. In some embodiments, the subject is a primate. In preferred embodiments, a subject is a human. Oligonucleotides may be employed as therapeutic moieties in the treatment of disease states in animals, including humans. Oligonucleotides can be useful therapeutic modalities that can be configured to be useful in treatment regimes for the treatment of cells, tissues and animals, especially humans.

For therapeutics, an animal, preferably a human, suspected of having a disease or disorder associated with low levels of an RNA or protein is treated by administering oligonucleotide in accordance with this invention. For example, in one non-limiting embodiment, the methods comprise the step of administering to the animal in need of treatment, a therapeutically effective amount of an oligonucleotide as described herein. Table 2 lists examples of diseases or conditions that may be treated by targeting mRNA transcripts with stabilizing oligonucleotides. In some embodiments, cells used in the methods disclosed herein may, for example, be cells obtained from a subject having one or more of the conditions listed in Table 2, or from a subject that is a disease model of one or more of the conditions listed in Table 2. In some embodiments, the disease or condition is a liver disease or condition. Exemplary liver diseases and conditions include, but are not limited to, Hepatitis (e.g. A, B, C), Fascioliasis, Alcoholic liver disease, Fatty liver disease, Cirrhosis, hepatocellular carcinoma, cholangiocarcinoma, angiosarcoma of the liver, hemangiosarcoma of the liver, Primary biliary cirrhosis, Primary sclerosing cholangitis, Budd-Chiari syndrome, hemochromatosis, Wilson's disease, alpha 1-antitrypsin deficiency, glycogen storage disease type II, Gilbert's syndrome, biliary atresia, Alagille syndrome, progressive familial intrahepatic cholestasis, Galactosemia, Hepatic Encephalopathy, hypercholesterolemia, biliary cirrhosis, Byler disease, cholestasis, cholestasis intrahepatic, dyslipidemia, Hemochromatosis (juvenile), and iron overload.

TABLE 2
Examples of diseases or conditions treatable with oligonucleotides targeting
associated mRNA.

| Gene | Disease or conditions |
|---|---|
| FXN | Friedreich′ s Ataxia |
| THRB | Thyroid hormone resistance, mixed dyslipidemia, dyslipidemia, |
| | hypercholesterolemia |
| NR1H4 | Byler disease, cholestasis, cholestasis intrahepatic, dyslipidemia, biliary cirrhosis |
| | primary, fragile x syndrome, hypercholesterolemia, atherosclerosis, biliary atresia |
| HAMP | Hemochromatosis (juvenile), hemochromatosis, iron overload, hereditary |
| | hemochromatosis, anemia, inflammation, thalassemia |
| APOA1 | Dyslipidemia (e.g. Hyperlipidemia) and atherosclerosis (e.g. coronary artery disease |
| | (CAD) and myocardial infarction (MI)) |

### Formulation, Delivery, and Dosing

The oligonucleotides described herein can be formulated for administration to a subject for treating a condition associated with decreased levels of expression of gene or instability or low stability of an RNA transcript that results in decreased levels of expression of a gene (e.g., decreased protein levels or decreased levels of functional RNAs, such as miRNAs, snoRNAs, lncRNAs, etc.). It should be understood that the formulations, compositions and methods can be practiced with any of the oligonucleotides disclosed herein.

The formulations may conveniently be presented in unit dosage form and may be prepared by any methods well known in the art of pharmacy. The amount of active ingredient (e.g., an oligonucleotide or compound of the invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g. tumor regression.

Pharmaceutical formulations of this invention can be prepared according to any method known to the art for the manufacture of pharmaceuticals. Such formulations can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

A formulated oligonucleotide composition can assume a variety of states. In some examples, the composition is at least partially crystalline, uniformly crystalline, and/or anhydrous (e.g., less than 80, 50, 30, 20, or 10% water). In another example, an oligonucleotide is in an aqueous phase, e.g., in a solution that includes water. The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, an oligonucleotide composition is formulated in a manner that is compatible with the intended method of administration.

In some embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

An oligonucleotide preparation can be formulated or administered (together or separately) in combination with another agent, e.g., another therapeutic agent or an agent that stabilizes an oligonucleotide, e.g., a protein that complexes with oligonucleotide. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, an oligonucleotide preparation includes another oligonucleotide, e.g., a second oligonucleotide that modulates expression of a second gene or a second oligonucleotide that modulates expression of the first gene. Still other preparation can include at least 3, 5, ten, twenty, fifty, or a hundred or more different oligonucleotide species. Such oligonucleotides can mediated gene expression with respect to a similar number of different genes. In one embodiment, an oligonucleotide preparation includes at least a second therapeutic agent (e.g., an agent other than an oligonucleotide).

Any of the formulations, excipients, vehicles, etc. disclosed herein may be adapted or used to facilitate delivery of synthetic RNAs (e.g., circularized synthetic RNAs) to a cell. Formulations, excipients, vehicles, etc. disclosed herein may be adapted or used to facilitate delivery of a synthetic RNA to a cell in vitro or in vivo. For example, a synthetic RNA (e.g., a circularized synthetic RNA) may be formulated with a nanoparticle, poly(lactic-co-glycolic acid) (PLGA) microsphere, lipidoid, lipoplex, liposome, polymer, carbohydrate (including simple sugars), cationic lipid, a fibrin gel, a fibrin hydrogel, a fibrin glue, a fibrin sealant, fibrinogen, thrombin, rapidly eliminated lipid nanoparticles (reLNPs) and combinations thereof. In some embodiments, a synthetic RNA may be delivered to a cell gymnotically. In some embodiments, oligonucleotides or synthetic RNAs may be conjugated with factors that facilitate delivery to cells. In some embodiments, a synthetic RNA or oligonucleotide used to circularize a synthetic RNA is conjugated with a carbohydrate, such as GalNac, or other targeting moiety.

### Route of Delivery

A composition that includes an oligonucleotide can be delivered to a subject by a variety of routes. Exemplary routes include: intravenous, intradermal, topical, rectal, parenteral, anal, intravaginal, intranasal, pulmonary, ocular. The term “therapeutically effective amount” is the amount of oligonucleotide present in the composition that is needed to provide the desired level of gene expression (e.g., by stabilizing RNA transcripts) in the subject to be treated to give the anticipated physiological response. The term “physiologically effective amount” is that amount delivered to a subject to give the desired palliative or curative effect. The term “pharmaceutically acceptable carrier” means that the carrier can be administered to a subject with no significant adverse toxicological effects to the subject.

An oligonucleotide molecules of the invention can be incorporated into pharmaceutical compositions suitable for administration. Such compositions typically include one or more species of oligonucleotide and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering an oligonucleotide in aerosol form. The vascular endothelial cells could be targeted by coating a balloon catheter with an oligonucleotide and mechanically introducing the oligonucleotide.

Topical administration refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy. In addition, iontophoresis (transfer of ionic solutes through biological membranes under the influence of an electric field), phonophoresis or sonophoresis (use of ultrasound to enhance the absorption of various therapeutic agents across biological membranes, notably the skin and the cornea), and optimization of vehicle characteristics relative to dose position and retention at the site of administration may be useful methods for enhancing the transport of topically applied compositions across skin and mucosal sites.

Both the oral and nasal membranes offer advantages over other routes of administration. For example, oligonucleotides administered through these membranes may have a rapid onset of action, provide therapeutic plasma levels, avoid first pass effect of hepatic metabolism, and avoid exposure of the oligonucleotides to the hostile gastrointestinal (GI) environment. Additional advantages include easy access to the membrane sites so that the oligonucleotide can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many agents. Further, the sublingual mucosa is convenient, acceptable and easily accessible.

A pharmaceutical composition of oligonucleotide may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity.

Compositions for oral administration include powders or granules, suspensions or solutions in water, syrups, slurries, emulsions, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Parenteral administration includes intravenous drip, subcutaneous, intraperitoneal or intramuscular injection, intrathecal or intraventricular administration. In some embodiments, parental administration involves administration directly to the site of disease (e.g. injection into a tumor).

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

Any of the oligonucleotides described herein can be administered to ocular tissue. For example, the compositions can be applied to the surface of the eye or nearby tissue, e.g., the inside of the eyelid. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers. An oligonucleotide can also be administered to the interior of the eye, and can be introduced by a needle or other delivery device which can introduce it to a selected area or structure.

Pulmonary delivery compositions can be delivered by inhalation by the patient of a dispersion so that the composition, preferably oligonucleotides, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self-contained. Dry powder dispersion devices, for example, deliver agents that may be readily formulated as dry powders. An oligonucleotide composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 μm in diameter preferably with a relatively uniform spheroidal shape distribution. More preferably the diameter is less than about 7.5 μm and most preferably less than about 5.0 μm. Usually the particle size distribution is between about 0.1 μm and about 5 μm in diameter, particularly about 0.3 μm to about 5 μm.

The term “dry” means that the composition has a moisture content below about 10% by weight (% w) water, usually below about 5% w and preferably less it than about 3% w. A dry composition can be such that the particles are readily dispersible in an inhalation device to form an aerosol.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Suitable pH adjusters or buffers include organic salts prepared from organic acids and bases, such as sodium citrate, sodium ascorbate, and the like; sodium citrate is preferred. Pulmonary administration of a micellar oligonucleotide formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Exemplary devices include devices which are introduced into the vasculature, e.g., devices inserted into the lumen of a vascular tissue, or which devices themselves form a part of the vasculature, including stents, catheters, heart valves, and other vascular devices. These devices, e.g., catheters or stents, can be placed in the vasculature of the lung, heart, or leg.

Other devices include non-vascular devices, e.g., devices implanted in the peritoneum, or in organ or glandular tissue, e.g., artificial organs. The device can release a therapeutic substance in addition to an oligonucleotide, e.g., a device can release insulin.

In one embodiment, unit doses or measured doses of a composition that includes oligonucleotide are dispensed by an implanted device. The device can include a sensor that monitors a parameter within a subject. For example, the device can include pump, e.g., and, optionally, associated electronics.

Tissue, e.g., cells or organs can be treated with an oligonucleotide, ex vivo and then administered or implanted in a subject. The tissue can be autologous, allogeneic, or xenogeneic tissue. Introduction of treated tissue, whether autologous or transplant, can be combined with other therapies. In some implementations, an oligonucleotide treated cells are insulated from other cells, e.g., by a semi-permeable porous barrier that prevents the cells from leaving the implant, but enables molecules from the body to reach the cells and molecules produced by the cells to enter the body. In one embodiment, the porous barrier is formed from alginate.

In one embodiment, a contraceptive device is coated with or contains an oligonucleotide. Exemplary devices include condoms, diaphragms, IUD (implantable uterine devices, sponges, vaginal sheaths, and birth control devices.

### Dosage

In one aspect, the invention features a method of administering an oligonucleotide (e.g., as a compound or as a component of a composition) to a subject (e.g., a human subject). In one embodiment, the unit dose is between about 10 mg and 25 mg per kg of bodyweight. In one embodiment, the unit dose is between about 1 mg and 100 mg per kg of bodyweight. In one embodiment, the unit dose is between about 0.1 mg and 500 mg per kg of bodyweight.

In some embodiments, the unit dose is more than 0.001, 0.005, 0.01, 0.05, 0.1, 0.5, 1, 2, 5, 10, 25, 50 or 100 mg per kg of bodyweight.

The defined amount can be an amount effective to treat or prevent a disease or disorder, e.g., a disease or disorder associated with low levels of an RNA or protein (e.g., in a liver cell of a human subject). The unit dose, for example, can be administered by injection (e.g., intravenous, hepatic intra-arterial, intraportal, subcutaneous, or intramuscular), an inhaled dose, or a topical application.

In some embodiments, the unit dose is administered daily. In some embodiments, less frequently than once a day, e.g., less than every 2, 4, 8 or 30 days. In another embodiment, the unit dose is not administered with a frequency (e.g., not a regular frequency). For example, the unit dose may be administered a single time. In some embodiments, the unit dose is administered more than once a day, e.g., once an hour, two hours, four hours, eight hours, twelve hours, etc.

In one embodiment, a subject is administered an initial dose and one or more maintenance doses of an oligonucleotide. The maintenance dose or doses are generally lower than the initial dose, e.g., one-half less of the initial dose. A maintenance regimen can include treating the subject with a dose or doses ranging from 0.0001 to 100 mg/kg of body weight per day, e.g., 100, 10, 1, 0.1, 0.01, 0.001, or 0.0001 mg per kg of bodyweight per day. The maintenance doses may be administered no more than once every 1, 5, 10, or 30 days. Further, the treatment regimen may last for a period of time which will vary depending upon the nature of the particular disease, its severity and the overall condition of the patient. In some embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the oligonucleotide may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some cases, a patient is treated with an oligonucleotide in conjunction with other therapeutic modalities.

Following successful treatment, it may be desirable to have the patient undergo maintenance therapy to prevent the recurrence of the disease state, wherein the compound of the invention is administered in maintenance doses, ranging from 0.0001 mg to 100 mg per kg of body weight.

The concentration of an oligonucleotide composition is an amount sufficient to be effective in treating or preventing a disorder or to regulate a physiological condition in humans. The concentration or amount of oligonucleotide administered will depend on the parameters determined for the agent and the method of administration, e.g. nasal, buccal, pulmonary. For example, nasal formulations may tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an oligonucleotide can include a single treatment or, preferably, can include a series of treatments. It will also be appreciated that the effective dosage of an oligonucleotide used for treatment may increase or decrease over the course of a particular treatment. For example, the subject can be monitored after administering an oligonucleotide composition. Based on information from the monitoring, an additional amount of an oligonucleotide composition can be administered.

Dosing is dependent on severity and responsiveness of the disease condition to be treated, with the course of treatment lasting from several days to several months, or until a cure is effected or a diminution of disease state is achieved. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models.

In one embodiment, the administration of an oligonucleotide composition is parenteral, e.g. intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The composition can be provided in measured doses or in a dispenser which delivers a metered dose. Selected modes of delivery are discussed in more detail below.

### Kits

In certain aspects of the invention, kits are provided, comprising a container housing a composition comprising an oligonucleotide. In some embodiments, the composition is a pharmaceutical composition comprising an oligonucleotide and a pharmaceutically acceptable carrier. In some embodiments, the individual components of the pharmaceutical composition may be provided in one container. Alternatively, it may be desirable to provide the components of the pharmaceutical composition separately in two or more containers, e.g., one container for oligonucleotides, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1. Oligonucleotide for Targeting 5′ and 3′ Ends of RNAs

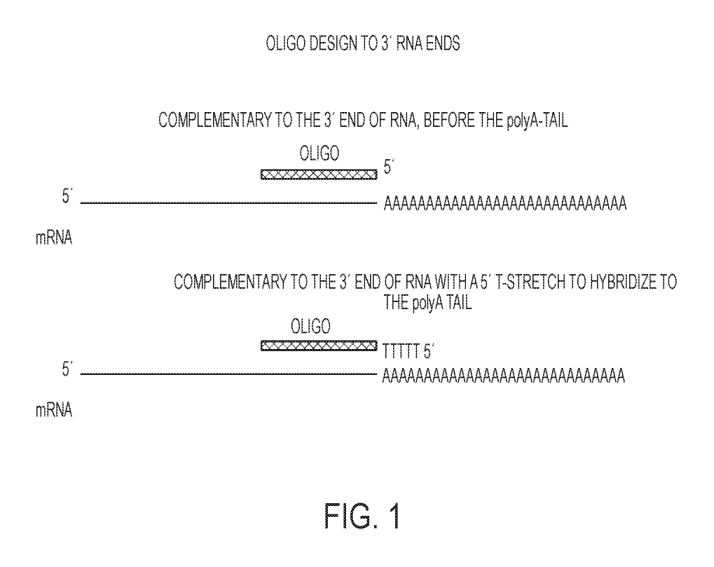

Several exemplary oligonucleotide design schemes are contemplated herein for increasing mRNA stability. With regard to oligonucleotides targeting the 3′ end of an RNA, at least two exemplary design schemes are contemplated. As a first scheme, an oligo nucleotide is designed to be complementary to the 3′ end of an RNA, before the poly-A tail (FIG. 1). As a second scheme, an oligonucleotide is designed to be complementary to the 3′ end of RNA with a 5′ poly-T region that hybridizes to a poly-A tail (FIG. 1).

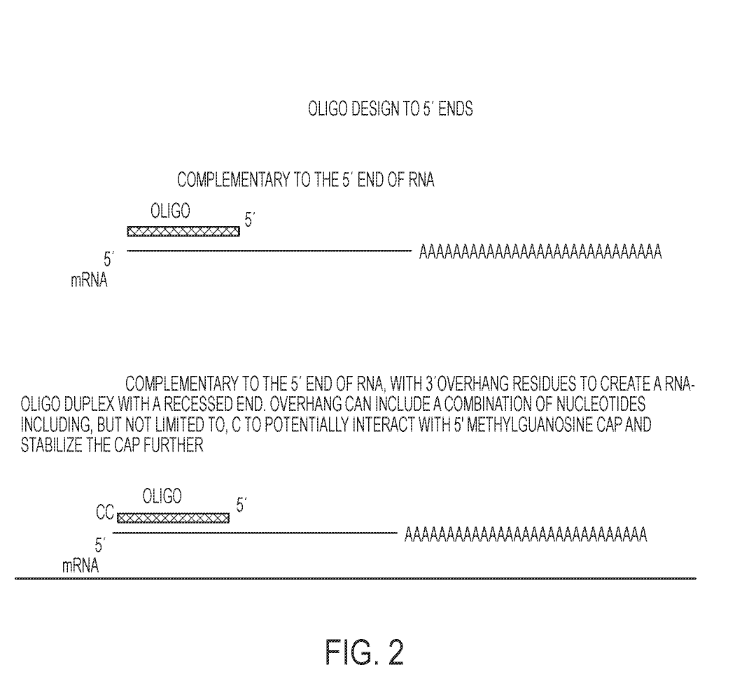

With regard to oligonucleotides targeting the 5′ end of an RNA, at least three exemplary design schemes are contemplated. For scheme one, an oligonucleotide is designed to be complementary to the 5′ end of RNA (FIG. 2). For scheme two, an oligonucleotide is designed to be complementary to the 5′ end of RNA and has a 3′ overhang to create a RNA-oligo duplex with a recessed end. In this example, the overhang is one or more C nucleotides, e.g., two Cs, which can potentially interact with a 5′ methylguanosine cap and stabilize the cap further (FIG. 2). The overhang could also potentially be another type of nucleotide, and is not limited to C. For scheme 3, an oligonucleotide is designed to include a loop region to stabilize 5′ RNA cap (FIG. 3A). FIG. 3A also shows an exemplary oligo for targeting 5′ and 3′ RNA ends. The example shows oligos that bind to a 5′ and 3′ RNA end to create a pseudo-circularized RNA (FIG. 3B).

An oligonucleotide designed as described in Example 1 may be tested for its ability to upregulate RNA by increasing mRNA stability using the methods outlined in Example 2.

### Example 2: Oligos for Targeting the 5′ and 3′ End of FXN, APOA1, THRB, HAMP and NR1H4

### Oligonucleotide Design

Oligonucleotides were designed to target the 5′ and 3′ ends of FXN, APOA1, THRB, HAMP and NR1H4 mRNA. The 3′ end oligonucleotides were designed by identifying putative mRNA 3′ ends using quantitative end analysis of poly-A tails as described previously (see, e.g., Ozsolak et al. Comprehensive Polyadenylation Site Maps in Yeast and Human Reveal Pervasive Alternative Polyadenylation. Cell. Volume 143, Issue 6, 2010, Pages 1018-1029). The 5′ end oligonucleotides were designed by identifying potential 5′ start sites using Cap analysis gene expression (CAGE) as previously described (see, e.g., Cap analysis gene expression for high-throughput analysis of transcriptional starting point and identification of promoter usage. Proc Natl Acad Sci USA. 100 (26): 15776-81. 2003-12-23 and Zhao, Xiaobei (2011). “Systematic Clustering of Transcription Start Site Landscapes”. PLoS ONE (Public Library of Science) 6 (8): e23409).

### Example 3: Oligos for Targeting Thyroid Hormone Receptor (TRβ)

Thyroid hormone receptor beta (TRβ) is a type 1 nuclear receptor that mediates the biological activities of triiodothyronine (T3) hormone. TRβ isoform 1 (TRβ1) is the predominant isoform in the liver and kidney and controls metabolism and mediates the cholesterol lowering effects of thyroid hormones. Cardiac effects of T3 are mediated by a related protein (TRα) in the heart. Selective activation of TRβ using T3 agonists has progressed to phase II clinical trials (Senese et al., (2014) Front Physiol 5, 1-7).

TR activation effects lipid homeostasis. Upregulated cholesterol clearance and disposal leads to an increase in the low-density lipoprotein receptor (independent of sterol regulatory element-binding proteins (SREBPs)) and an increase in bile acid synthesis and elimination through human cholesterol 7alpha-hydroxylase (CYP7A1). Stimulation of the reverse cholesterol transport pathway results in increased levels of the apolipoprotein A1 (ApoA) component of high-density lipoprotein, scavenger receptor class B member 1 (SRB1), cholesterylester transfer protein (CETP), and hepatic triglyceride lipase (HTGL). This also results in increased Lecithin-Cholesterol Acyltransferase (LCAT) activity. Stimulated fatty acid beta-oxidation leads to an increase in carnitine palmitoyltransferase I (CPT1) and a decrease in SREBP1-c. Stimulation of hepatic energy expenditure leads to an increase in uncoupling protein (UCP), futile cycling, and mitochondrial biogenesis. Overall, the effect of thyroid receptor activation affects lipid homeostasis through a decrease in levels of very low density lipoprotein C (VLDL-C), low density lipoprotein C (LDL-C), lipoprotein(a), Apolipoprotein B, and triglycerides.

In some embodiments, non-alcoholic fatty liver disease (NAFLD) and nonalcoholic steatohepatitis (NASH) are the primary indications for TRβ upregulation. NAFLD represents steatosis where fat is greater than 5% of hepatocytes. NASH represents steatohepatitis, a combination of steatosis and inflammation. TRβ-selective agonists have been shown to prevent or improve metabolic complications arising from high-fat diet, including NAFLD.

Human trials of thyroid hormone receptor (THR) modulators have been pursued (Ayers et al., J. Endocrinol Diabetes Obes 2(3):1042 (2014)). Phase 1a trials of GC-1 (sobetirome) aim to reduce LDL cholesterol (LDL-C) for treatment of dyslipidemias, hypercholesterolemia, obesity, and NAFLD. Phase 1b trials of MD07811 aim to reduce LDL-C and triglyceride (TG) levels for the treatment of dyslipidemias and hypercholesterolemia. Phase II trials of KB-2115 (Eprotirome) aim to reduce LDL-C, lipoprotein(a), and TG through statin synergy for treatment of dyslipidemias, hypercholesterolemia, and familial hypercholesterolemia (FH). Phase 1b trials of MGL3196 aim to reduce LDLC and TG for treatment of dyslipidemias, hypercholesterolemia, NAFLD, and FH. Phase 1b trials of DITPA aim to reduce LDL-C through statin synergy for the treatment of dyslipidemias, hypercholesterolemia, and heart failure. In some embodiments, oligonucleotides provided herein can be used in combination with or in place of the THR modulators described above.

Activation of TRβ leading to lowering of serum LDL-C and triglycerides has been demonstrated with TRβ agonists in multiple rodent models (Erion M D et al. 2007. Proc Natl Acad Sci 104:15490-15495). Activation of TRβ leading to lowering of liver triglycerides has been demonstrated with TRβ agonists in multiple rodent models of NAFLD (Cable E et al. 2009. Hepatology 49: 407-417). Activation of TRβ in NHP led to reduced serum LDL-C as well as reduced Lp(a) levels. Lp(a) is an independent, primate-specific risk factor for atherosclerosis (Tancevski I et al. 2009. J Lipid Res 50:938-944). Adenovirus mediated TRβ expression in mice (5-fold over-expression) produces expected effects on lipid metabolism and activates downstream genes so T3 is not limiting. In some embodiments, TRβ upregulation has the same or similar effect as TRβ activation. In some embodiments, TRβ upregulation increases sensitivity to endogenous T3. Accordingly, in some embodiments, upregulation of endogenous TRβ in the liver using methods disclosed herein has a beneficial effect on liver triglycerides as well as serum lipid profiles.

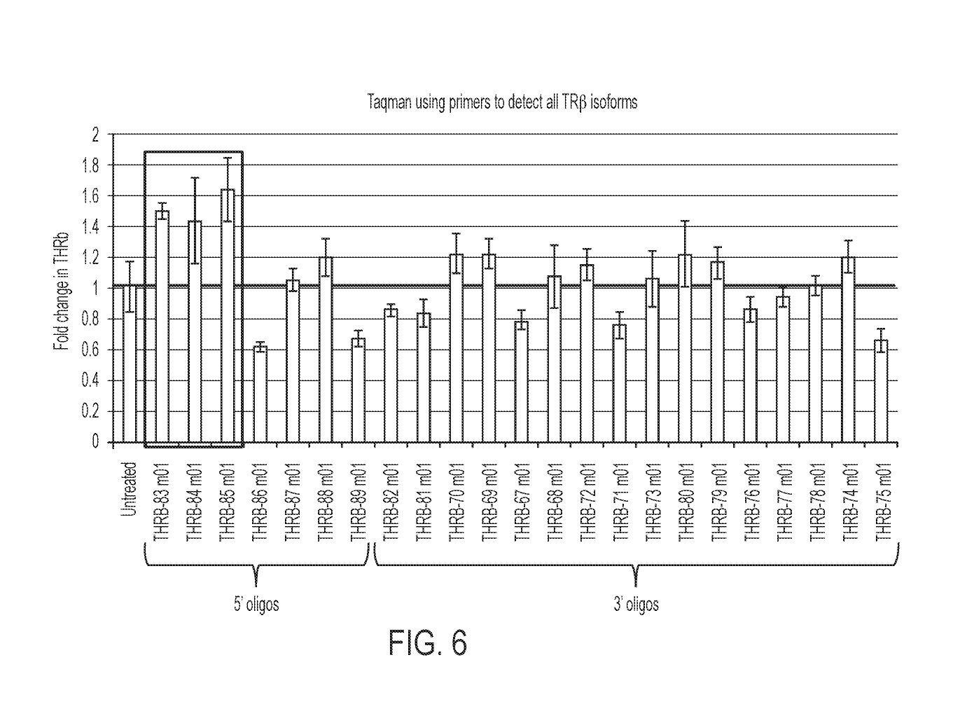

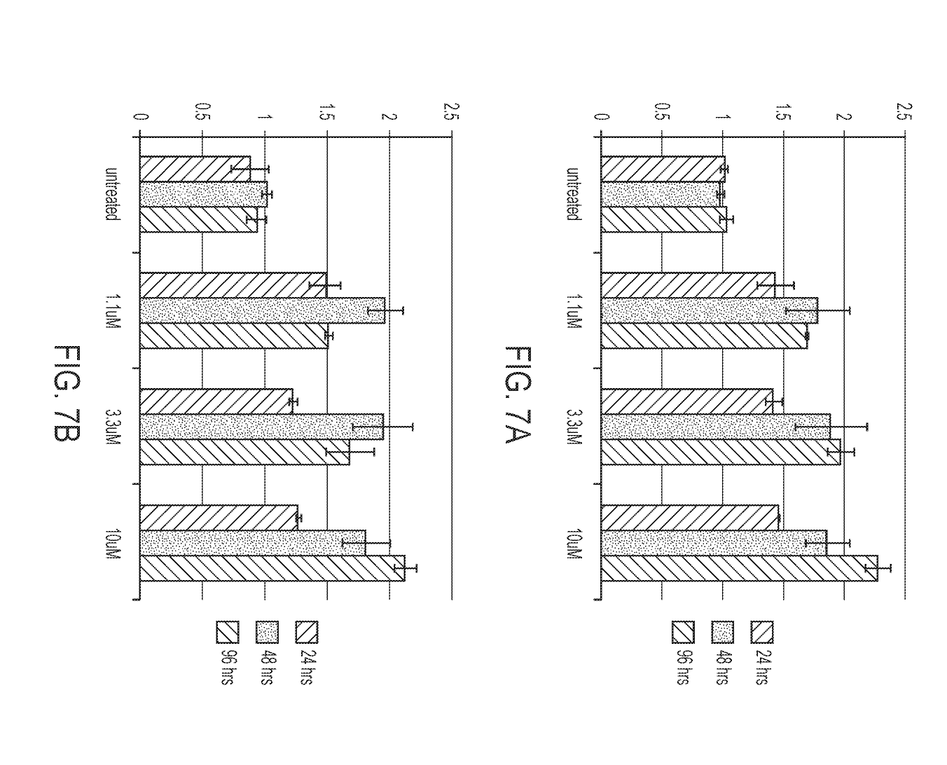

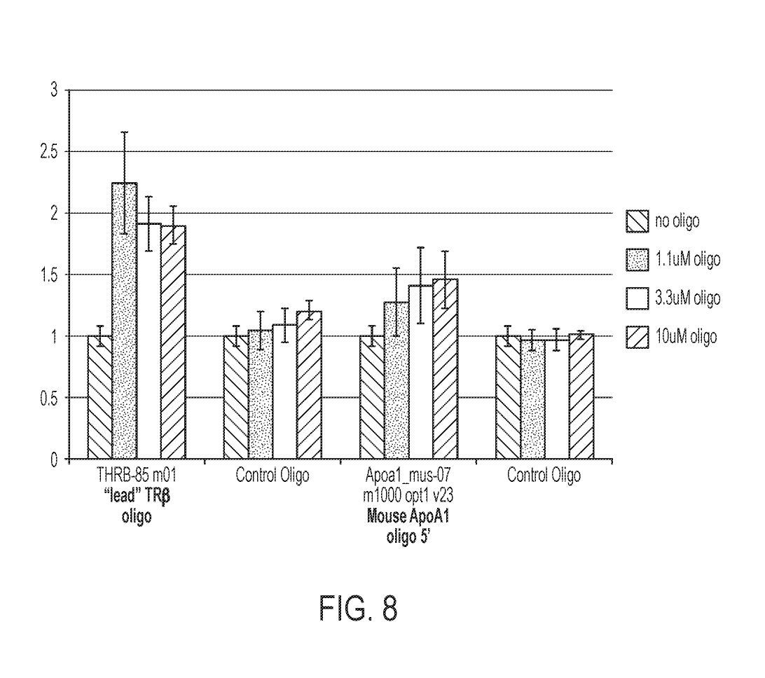

A primary screen of human THRβ end-targeting oligonucleotides was conducted. FIG. 6 illustrates that at least three 5′ oligonucleotides increase THRβ mRNA. In some embodiments, 5′/3′ combinations are similarly active. A secondary screen of human THRβ 5′ end-targeting oligonucleotide THRB-85 m01 illustrates concentration-dependent upregulation of TRβ−1 mRNA with a single oligo. In some embodiments, upregulation is time-dependent only with respect to the highest oligonucleotide concentration, which may suggest a need to saturate a non-productive uptake pathway (FIGS. 7A-7B). A 5 day treatment in a donor shows that THRB-85 m01 activity is specific to the oligonucleotide treatment (FIG. 8).

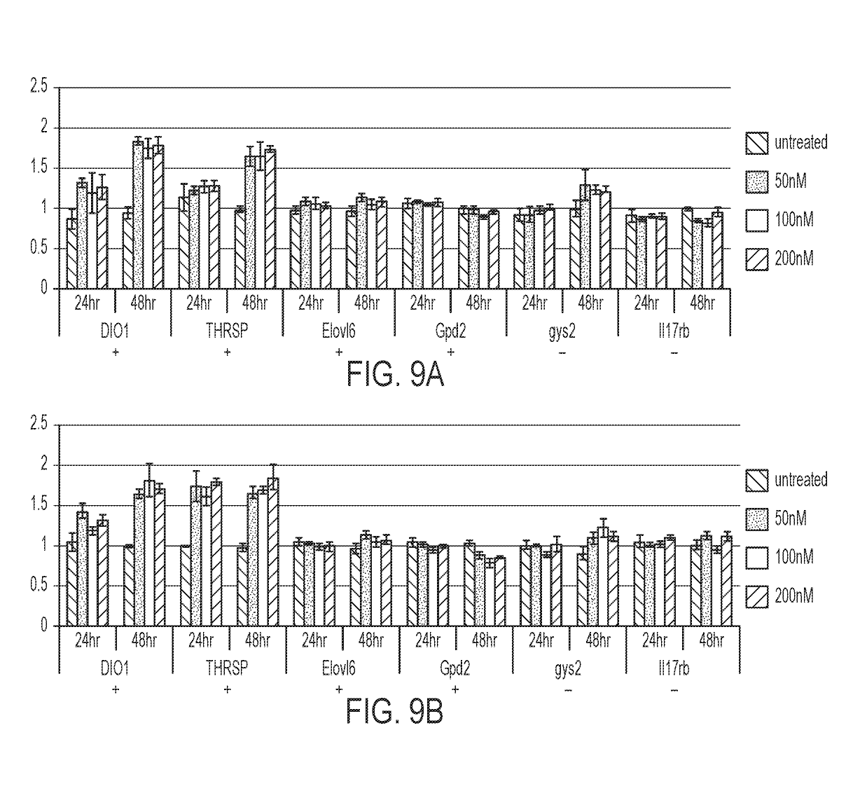

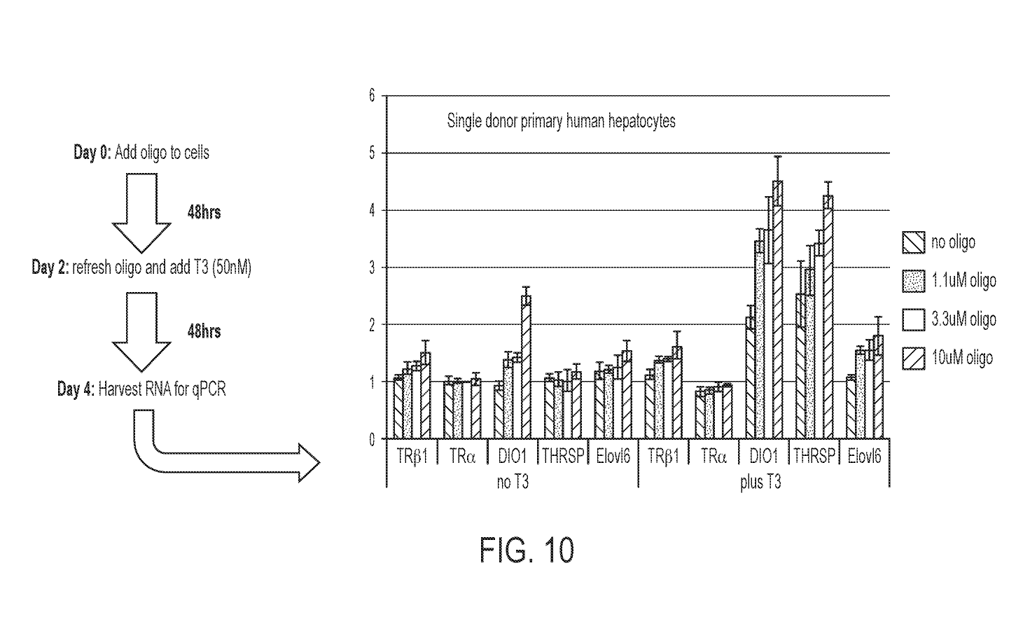

Downstream genes can be used as a readout for TRβ protein activity. Downstream gene analysis shows that human primary hepatocytes are responsive to T3 treatment in both single and pooled donors (FIGS. 9A-9B). Through combining THRB-85 m01 oligonucleotide with T3 treatment, downstream genes can be used as a readout for TRβ upregulation. A slight upregulation of TRβ1 mRNA is evident (usually 1.5-2× upregulated with THRB-85 m01) and there is no upregulation of TRα. T3 treatment indicates dose-sensitive, T3-responsive effect of the oligonucleotide on downstream genes, and in particular, the thyroid hormone responsive (THRSP) gene (FIG. 10).

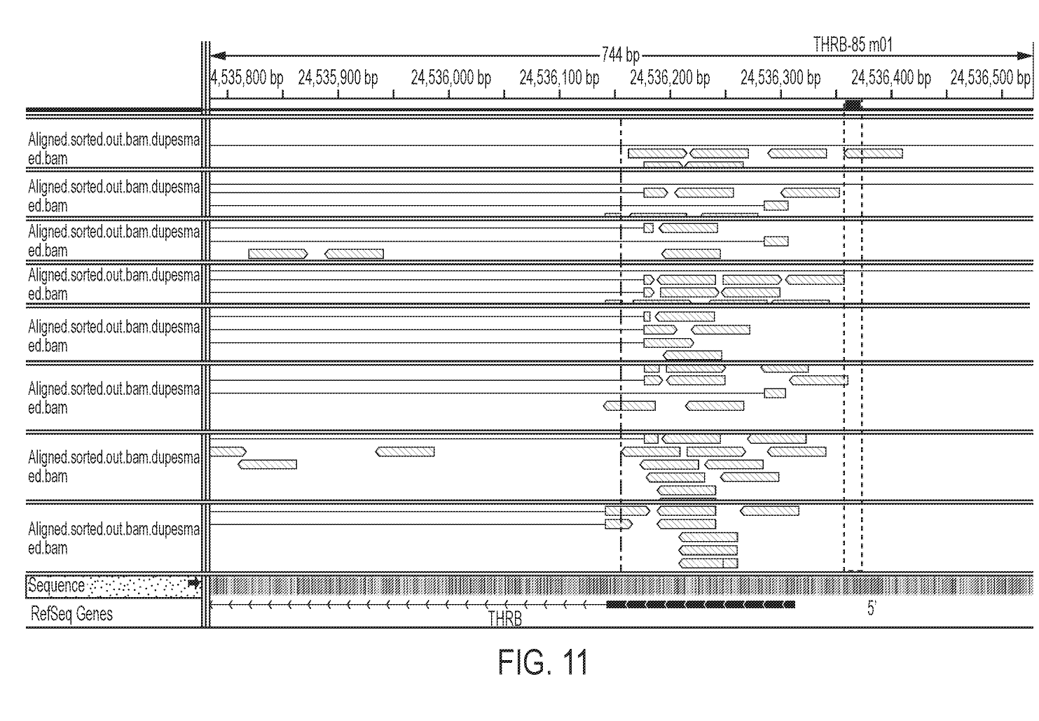

Alignment of THRB-85 m01 to TRβ indicates that THRB-85 m01 overlaps with the 5′ end of the transcript (FIG. 11).

Apolipoprotein A-I (ApoA1) mRNA levels after a 5 day oligonucleotide treatment of mouse primary hepatocytes illustrates that 5′ and 3′ oligonucleotide combinations are not significantly more active than singles (FIG. 14).

The 5′ stabilization oligonucleotide THRB-85 m01 appears to upregulate TRβ1 mRNA approximately twice as much as the control. THRB-85 m01 can be mapped to the 5′ end of TRβ1 identified by RACE and RNASeq. Cultured human hepatocytes can respond to T3 hormone treatment by activating known TRβ target genes and THRB-85 m01 treatment enhances this effect.

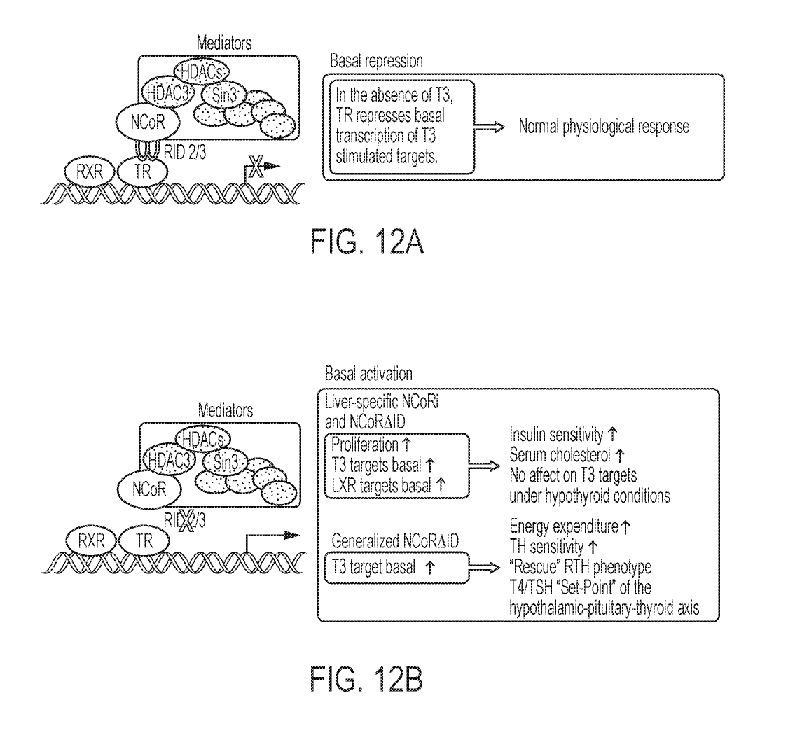

A detailed mechanism for THRβ activity suggests that there are two different pathways for basal repression and basal activation. For basal repression, unliganded TR is bound to DNA but transcription is inhibited by NCoR and its associated HDAC complex (FIG. 12A). However, NCoR with a deleted Interaction Domain (RID2/3) relieves repression and results in activated basal transcription (FIG. 12B).

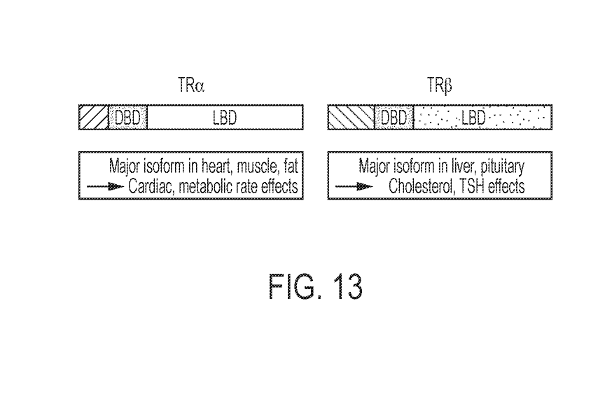

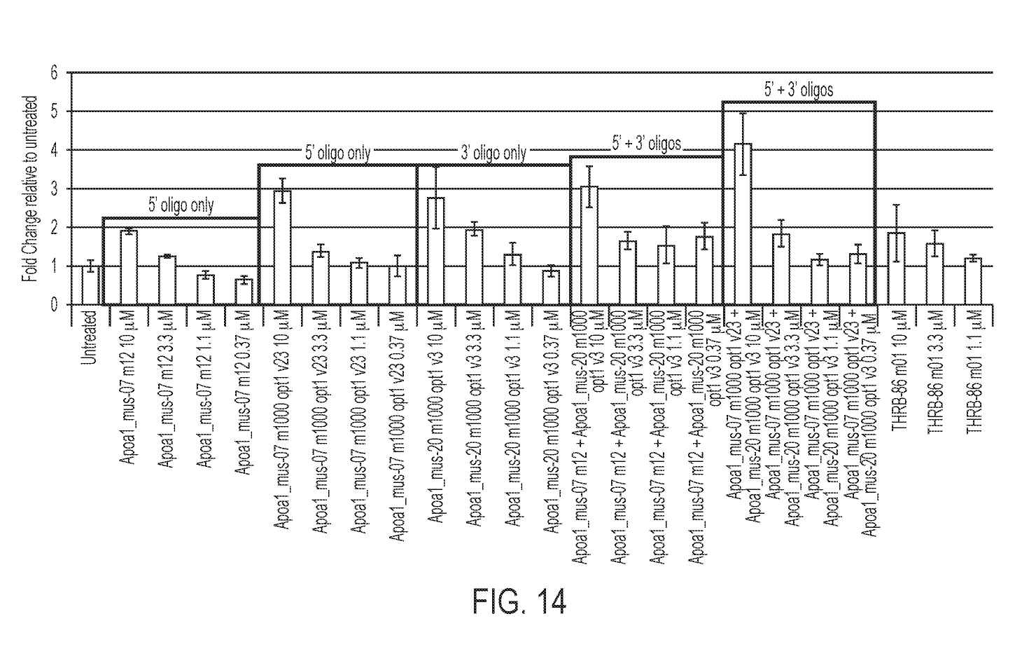

Thyroid hormone mediates its physiological effects by binding to specific nuclear receptors. TR receptors are ubiquitously expressed throughout body and there are two major isoforms, TRα and TRβ, with differential expression across tissues. TRα is the major isoform in the heart, muscle, and fat and has cardiac and metabolic rate effects. TRβ is the major isoform in the liver and pituitary and has effects on cholesterol and thyroid-stimulating hormone. There is one amino acid different in ligand binding domain (LBD) between the isoforms (FIG. 13).

The sequence and structure of each oligonucleotide is shown in Table 3. Table 4 provides a description of the nucleotide analogs, modifications and intranucleotide linkages used for certain oligonucleotides tested and described in Tables 3 and 5.

TABLE 3
Oligonucleotides targeting 5′ and 3′ ends of FXN, APOA1, THRB, HAMP and
NR1H4

| | | | SEQ | | |
|---|---|---|---|---|---|
| Oligo | | | ID | | |
| Name | Gene | Organism | NO. | Base Sequence | Formatted Sequence |
| THRB- | THRB | human | 1 | CTGTTATAAGCTTTT | InamCs; omeUs; InaGs; omeUs; InaTs; |
| 67 | | | | | omeAs; InaTs; omeAs; InaAs; omeGs; |
| m01 | | | | | InamCs; omeUs; InaTs; omeUs; InaT- |
| | | | | | Sup |
| THRB- | THRB | human | 2 | GTTATAAGCTTTTTC | InaGs; omeUs; InaTs; omeAs; InaTs; |
| 68 | | | | | omeAs; InaAs; omeGs; InamCs; omeUs; |
| m01 | | | | | InaTs; omeUs; InaTs; omeUs; InamC-Sup |
| THRB- | THRB | human | 3 | TATCTGTTATAAGCT | InaTs; omeAs; InaTs; omeCs; InaTs; |
| 69 | | | | | omeGs; InaTs; omeUs; InaAs; omeUs; |
| m01 | | | | | InaAs; omeAs; InaGs; omeCs; InaT-Sup |
| THRB- | THRB | human | 4 | AGTAGATGTTTATTT | InaAs; omeGs; InaTs; omeAs; InaGs; |
| 70 | | | | | omeAs; InaTs; omeGs; InaTs; omeUs; |
| m01 | | | | | InaTs; omeAs; InaTs; omeUs; InaT-Sup |
| THRB- | THRB | human | 5 | TAGGCAAAGGAATAG | InaTs; omeAs; InaGs; omeGs; InamCs; |
| 71 | | | | | omeAs; InaAs; omeAs; InaGs; omeGs; |
| m01 | | | | | InaAs; omeAs; InaTs; omeAs; InaG-Sup |
| THRB- | THRB | human | 6 | GGTAGGCAAAGGAAT | InaGs; omeGs; InaTs; omeAs; InaGs; |
| 72 | | | | | omeGs; InamCs; omeAs; InaAs; omeAs; |
| m01 | | | | | InaGs; omeGs; InaAs; omeAs; InaT-Sup |
| THRB- | THRB | human | 7 | GGCAAAGGAATAGTT | InaGs; omeGs; InamCs; omeAs; InaAs; |
| 73 | | | | | omeAs; InaGs; omeGs; InaAs; omeAs; |
| m01 | | | | | InaTs; omeAs; InaGs; omeUs; InaT-Sup |
| THRB- | THRB | human | 8 | GAAATGACACCCAGT | InaGs; omeAs; InaAs; omeAs; InaTs; |
| 74 | | | | | omeGs; InaAs; omeCs; InaAs; omeCs; |
| m01 | | | | | InamCs; omeCs; InaAs; omeGs; InaT-Sup |
| THRB- | THRB | human | 9 | AAATGACACCCAGTA | InaAs; omeAs; InaAs; omeUs; InaGs; |
| 75 | | | | | omeAs; InamCs; omeAs; InamCs; omeCs; |
| m01 | | | | | InamCs; omeAs; InaGs; omeUs; InaA-Sup |
| THRB- | THRB | human | 10 | GGCAATGGAATGAAA | InaGs; omeGs; InamCs; omeAs; InaAs; |
| 76 | | | | | omeUs; InaGs; omeGs; InaAs; omeAs; |
| m01 | | | | | InaTs; omeGs; InaAs; omeAs; InaA-Sup |
| THRB- | THRB | human | 11 | CAATGGAATGAAATG | InamCs; omeAs; InaAs; omeUs; InaGs; |
| 77 | | | | | omeGs; InaAs; omeAs; InaTs; omeGs; |
| m01 | | | | | InaAs; omeAs; InaAs; omeUs; InaG-Sup |
| THRB- | THRB | human | 12 | ATGGAATGAAATGAC | InaAs; omeUs; InaGs; omeGs; InaAs; |
| 78 | | | | | omeAs; InaTs; omeGs; InaAs; omeAs; |
| m01 | | | | | InaAs; omeUs; InaGs; omeAs; InamC-Sup |
| THRB- | THRB | human | 13 | GTTTCAAGTACCCGC | InaGs; omeUs; InaTs; omeUs; InamCs; |
| 79 | | | | | omeAs; InaAs; omeGs; InaTs; omeAs; |
| m01 | | | | | InamCs; omeCs; InamCs; omeGs; InamC- |
| | | | | | Sup |
| THRB- | THRB | human | 14 | GACCGGAGAACGAAA | InaGs; omeAs; InamCs; omeCs; InaGs; |
| 80 | | | | | omeGs; InaAs; omeGs; InaAs; omeAs; |
| m01 | | | | | InamCs; omeGs; InaAs; omeAs; InaA-Sup |
| THRB- | THRB | human | 15 | CTTTGGAAGGTGTTT | InamCs; omeUs; InaTs; omeUs; InaGs; |
| 81 | | | | | omeGs; InaAs; omeAs; InaGs; omeGs; |
| m01 | | | | | InaTs; omeGs; InaTs; omeUs; InaT-Sup |
| THRB- | THRB | human | 16 | TTTCTTTGGAAGGTG | InaTs; omeUs; InaTs; omeCs; InaTs; |
| 82 | | | | | omeUs; InaTs; omeGs; InaGs; omeAs; |
| m01 | | | | | InaAs; omeGs; InaGs; omeUs; InaG-Sup |
| THRB- | THRB | human | 17 | AGTTAATCCCCGCCG | InaAs; omeGs; InaTs; omeUs; InaAs; |
| 83 | | | | | omeAs; InaTs; omeCs; InamCs; omeCs; |
| m01 | | | | | InamCs; omeGs; InamCs; omeCs; InaG- |
| | | | | | Sup |
| THRB- | THRB | human | 18 | TCCTGCAAAATGTCA | InaTs; omeCs; InamCs; omeUs; InaGs; |
| 84 | | | | | omeCs; InaAs; omeAs; InaAs; omeAs; |
| m01 | | | | | InaTs; omeGs; InaTs; omeCs; InaA-Sup |
| THRB- | THRB | human | 19 | CCCCGCAGTCTCCAC | InamCs; omeCs; InamCs; omeCs; InaGs; |
| 85 | | | | | omeCs; InaAs; omeGs; InaTs; omeCs; |
| m01 | | | | | InaTs; omeCs; InamCs; omeAs; InamC- |
| | | | | | Sup |
| THRB- | THRB | human | 20 | TCTCCACCCTCCTCC | InaTs; omeCs; InaTs; omeCs; InamCs; |
| 86 | | | | | omeAs; InamCs; omeCs; InamCs; omeUs; |
| m01 | | | | | InamCs; omeCs; InaTs; omeCs; InamC- |
| | | | | | Sup |
| THRB- | THRB | human | 21 | GAGCGCCGGCGACTG | InaGs; omeAs; InaGs; omeCs; InaGs; |
| 87 | | | | | omeCs; InamCs; omeGs; InaGs; omeCs; |
| m01 | | | | | InaGs; omeAs; InamCs; omeUs; InaG- |
| | | | | | Sup |
| THRB- | THRB | human | 22 | AGGATGTGCGCCTTC | InaAs; omeGs; InaGs; omeAs; InaTs; |
| 88 | | | | | omeGs; InaTs; omeGs; InamCs; omeGs; |
| m01 | | | | | InamCs; omeCs; InaTs; omeUs; InamC- |
| | | | | | Sup |
| THRB- | THRB | human | 23 | GGCGCAGCGAGGAA | InaGs; omeGs; InamCs; omeGs; InamCs; |
| 89 | | | | | omeAs; InaGs; omeCs; InaGs; omeAs; |
| m01 | | | | | InaGs; omeGs; InaAs; InaA-Sup |
| THRB- | THRB | human | 24 | TTTCACTGACATCTC | InaTs; omeUs; InaTs; omeCs; InaAs; |
| 90 | | | | | omeCs; InaTs; omeGs; InaAs; omeCs; |
| m01 | | | | | InaAs; omeUs; InaCs; omeUs; InaC-Sup |
| HAMP- | HAMP | human | 25 | GGTGGTCTGAGCCCC | InaGs; omeGs; InaTs; omeGs; InaGs; |
| 01 | | | | | omeUs; InaCs; omeUs; InaGs; omeAs; |
| m01 | | | | | InaGs; omeCs; InaCs; omeCs; InaC-Sup |
| HAMP- | HAMP | human | 26 | GGGCCTGCCAGGGGA | InaGs; omeGs; InaGs; omeCs; InaCs; |
| 02 | | | | | omeUs; InaGs; omeCs; InaCs; omeAs; |
| m01 | | | | | InaGs; omeGs; InaGs; omeGs; InaA-Sup |
| HAMP- | HAMP | human | 27 | ACCGAGTGACAGTCG | InaAs; omeCs; InaCs; omeGs; InaAs; |
| 03 | | | | | omeGs; InaTs; omeGs; InaAs; omeCs; |
| m01 | | | | | InaAs; omeGs; InaTs; omeCs; InaG-Sup |
| HAMP- | HAMP | human | 28 | GTCTGGGACCGAGTG | InaGs; omeUs; InaCs; omeUs; InaGs; |
| 04 | | | | | omeGs; InaGs; omeAs; InaCs; omeCs; |
| m01 | | | | | InaGs; omeAs; InaGs; omeUs; InaG-Sup |
| HAMP- | HAMP | human | 29 | TGGGACCGAGTGACA | InaTs; omeGs; InaGs; omeGs; InaAs; |
| 05 | | | | | omeCs; InaCs; omeGs; InaAs; omeGs; |
| m01 | | | | | InaTs; omeGs; InaAs; omeCs; InaA-Sup |
| HAMP- | HAMP | human | 30 | GCAGAGGTGTGTTCA | InaGs; omeCs; InaAs; omeGs; InaAs; |
| 06 | | | | | omeGs; InaGs; omeUs; InaGs; omeUs; |
| m01 | | | | | InaGs; omeUs; InaTs; omeCs; InaA-Sup |
| HAMP- | HAMP | human | 31 | CGGCAGAGGTGTGTT | InaCs; omeGs; InaGs; omeCs; InaAs; |
| 07 | | | | | omeGs; InaAs; omeGs; InaGs; omeUs; |
| m01 | | | | | InaGs; omeUs; InaGs; omeUs; InaT-Sup |
| HAMP- | HAMP | human | 32 | GGGCAGACGGGGTCA | InaGs; omeGs; InaGs; omeCs; InaAs; |
| 08 | | | | | omeGs; InaAs; omeCs; InaGs; omeGs; |
| m01 | | | | | InaGs; omeGs; InaTs; omeCs; InaA-Sup |
| HAMP- | HAMP | human | 33 | CTCTGGTTTGGAAAA | InaCs; omeUs; InaCs; omeUs; InaGs; |
| 09 | | | | | omeGs; InaTs; omeUs; InaTs; omeGs; |
| m01 | | | | | InaGs; omeAs; InaAs; omeAs; InaA-Sup |
| HAMP- | HAMP | human | 34 | CTGGTTTGGAAAACA | InaCs; omeUs; InaGs; omeGs; InaTs; |
| 10 | | | | | omeUs; InaTs; omeGs; InaGs; omeAs; |
| m01 | | | | | InaAs; omeAs; InaAs; omeCs; InaA-Sup |
| HAMP- | HAMP | human | 35 | GTTTGGAAAACAAAA | InaGs; omeUs; InaTs; omeUs; InaGs; |
| 11 | | | | | omeGs; InaAs; omeAs; InaAs; omeAs; |
| m01 | | | | | InaCs; omeAs; InaAs; omeAs; InaA-Sup |
| HAMP- | HAMP | human | 36 | GGAAAACAAAAGAAC | InaGs; omeGs; InaAs; omeAs; InaAs; |
| 12 | | | | | omeAs; InaCs; omeAs; InaAs; omeAs; |
| m01 | | | | | InaAs; omeGs; InaAs; omeAs; InaC-Sup |
| HAMP- | HAMP | human | 37 | GAAAACAAAAGAACC | InaGs; omeAs; InaAs; omeAs; InaAs; |
| 13 | | | | | omeCs; InaAs; omeAs; InaAs; omeAs; |
| m01 | | | | | InaGs; omeAs; InaAs; omeCs; InaC-Sup |
| HAMP- | HAMP | human | 38 | TTTGGAAAACAAAAG | InaTs; omeUs; InaTs; omeGs; InaGs; |
| 14 | | | | | omeAs; InaAs; omeAs; InaAs; omeCs; |
| m01 | | | | | InaAs; omeAs; InaAs; omeAs; InaG-Sup |
| HAMP- | HAMP | human | 39 | TGGAAAACAAAAGAA | InaTs; omeGs; InaGs; omeAs; InaAs; |
| 15 | | | | | omeAs; InaAs; omeCs; InaAs; omeAs; |
| m01 | | | | | InaAs; omeAs; InaGs; omeAs; InaA-Sup |
| HAMP- | HAMP | human | 40 | TCTGGGGCAGCAGGA | InaTs; omeCs; InaTs; omeGs; InaGs; |
| 16 | | | | | omeGs; InaGs; omeCs; InaAs; omeGs; |
| m01 | | | | | InaCs; omeAs; InaGs; omeGs; InaA-Sup |
| NR1H | NR1 | human | 41 | ATAGAAAGGAACCTT | InaAs; omeUs; InaAs; omeGs; InaAs; |
| 4-01 | H4 | | | | omeAs; InaAs; omeGs; InaGs; omeAs; |
| m01 | | | | | InaAs; omeCs; InaCs; omeUs; InaT-Sup |
| NR1H | NR1 | human | 42 | TAGAAAGGAACCTTG | InaTs; omeAs; InaGs; omeAs; InaAs; |
| 4-02 | H4 | | | | omeAs; InaGs; omeGs; InaAs; omeAs; |
| m01 | | | | | InaCs; omeCs; InaTs; omeUs; InaG-Sup |
| NR1H | NR1 | human | 43 | ATTAACAATCCTTCC | InaAs; omeUs; InaTs; omeAs; InaAs; |
| 4-03 | H4 | | | | omeCs; InaAs; omeAs; InaTs; omeCs; |
| m01 | | | | | InaCs; omeUs; InaTs; omeCs; InaC-Sup |
| NR1H | NR1 | human | 44 | CTTCCCTGCTAAATG | InaCs; omeUs; InaTs; omeCs; InaCs; |
| 4-04 | H4 | | | | omeCs; InaTs; omeGs; InaCs; omeUs; |
| m01 | | | | | InaAs; omeAs; InaAs; omeUs; InaG-Sup |
| NR1H | NR1 | human | 45 | CCCTGCTAAATGATA | InaCs; omeCs; InaCs; omeUs; InaGs; |
| 4-05 | H4 | | | | omeCs; InaTs; omeAs; InaAs; omeAs; |
| m01 | | | | | InaTs; omeGs; InaAs; omeUs; InaA-Sup |
| NR1H | NR1 | human | 46 | TGATATAAACATAGA | InaTs; omeGs; InaAs; omeUs; InaAs; |
| 4-06 | H4 | | | | omeUs; InaAs; omeAs; InaAs; omeCs; |
| m01 | | | | | InaAs; omeUs; InaAs; omeGs; InaA-Sup |
| NR1H | NR1 | human | 47 | TCCCCGGGACTGAAC | InaTs; omeCs; InaCs; omeCs; InaCs; |
| 4-07 | H4 | | | | omeGs; InaGs; omeGs; InaAs; omeCs; |
| m01 | | | | | InaTs; omeGs; InaAs; omeAs; InaC-Sup |
| NR1H | NR1 | human | 48 | TACAACTTCTTTGAAT | InaTs; omeAs; InaCs; omeAs; InaAs; |
| 4-08 | H4 | | | | omeCs; InaTs; omeUs; InaCs; omeUs; |
| m01 | | | | | InaTs; omeUs; InaGs; omeAs; InaAs; |
| | | | | | InaT-Sup |
| NR1H | NR1 | human | 49 | TCTATCACTTCCCCG | InaTs; omeCs; InaTs; omeAs; InaTs; |
| 4-09 | H4 | | | | omeCs; InaAs; omeCs; InaTs; omeUs; |
| m01 | | | | | InaCs; omeCs; InaCs; omeCs; InaG-Sup |
| NR1H | NR1 | human | 50 | TCCTGGGCACCCGTA | InaTs; omeCs; InaCs; omeUs; InaGs; |
| 4-10 | H4 | | | | omeGs; InaGs; omeCs; InaAs; omeCs; |
| m01 | | | | | InaCs; omeCs; InaGs; omeUs; InaA-Sup |
| NR1H | NR1 | human | 51 | TTTCTGTACACATCA | InaTs; omeUs; InaTs; omeCs; InaTs; |
| 4-11 | H4 | | | | omeGs; InaTs; omeAs; InaCs; omeAs; |
| m01 | | | | | InaCs; omeAs; InaTs; omeCs; InaA-Sup |
| NR1H | NR1 | human | 52 | ACAGCCACTGAAAAT | InaAs; omeCs; InaAs; omeGs; InaCs; |
| 4-12 | H4 | | | | omeCs; InaAs; omeCs; InaTs; omeGs; |
| m01 | | | | | InaAs; omeAs; InaAs; omeAs; InaT-Sup |
| NR1H | NR1 | human | 53 | TTCACAGCCACTGAA | InaTs; omeUs; InaCs; omeAs; InaCs; |
| 4-13 | H4 | | | | omeAs; InaGs; omeCs; InaCs; omeAs; |
| m01 | | | | | InaCs; omeUs; InaGs; omeAs; InaA-Sup |
| NR1H | NR1 | human | 54 | TAAAGAAATGAGTTT | InaTs; omeAs; InaAs; omeAs; InaGs; |
| 4-14 | H4 | | | | omeAs; InaAs; omeAs; InaTs; omeGs; |
| m01 | | | | | InaAs; omeGs; InaTs; omeUs; InaT-Sup |
| NR1H | NR1 | human | 55 | ACATTTAAAGAAATG | InaAs; omeCs; InaAs; omeUs; InaTs; |
| 4-15 | H4 | | | | omeUs; InaAs; omeAs; InaAs; omeGs; |
| m01 | | | | | InaAs; omeAs; InaAs; omeUs; InaG-Sup |
| NR1H | NR1 | human | 56 | AAGAAATGAGTTTGT | InaAs; omeAs; InaGs; omeAs; InaAs; |
| 4-16 | H4 | | | | omeAs; InaTs; omeGs; InaAs; omeGs; |
| m01 | | | | | InaTs; omeUs; InaTs; omeGs; InaT-Sup |
| NR1H | NR1 | human | 57 | TGCCATTATGTTTGC | InaTs; omeGs; InaCs; omeCs; InaAs; |
| 4-17 | H4 | | | | omeUs; InaTs; omeAs; InaTs; omeGs; |
| m01 | | | | | InaTs; omeUs; InaTs; omeGs; InaC-Sup |
| NR1H | NR1 | human | 58 | TCCTGTTGCCATTAT | InaTs; omeCs; InaCs; omeUs; InaGs; |
| 4-18 | H4 | | | | omeUs; InaTs; omeGs; InaCs; omeCs; |
| m01 | | | | | InaAs; omeUs; InaTs; omeAs; InaT-Sup |
| NR1H | NR1 | human | 59 | GAAAATCCTGTTGCC | InaGs; omeAs; InaAs; omeAs; InaAs; |
| 4-19 | H4 | | | | omeUs; InaCs; omeCs; InaTs; omeGs; |
| m01 | | | | | InaTs; omeUs; InaGs; omeCs; InaC-Sup |
| NR1H | NR1 | human | 60 | CTGTTGCCATTATGT | InaCs; omeUs; InaGs; omeUs; InaTs; |
| 4-20 | H4 | | | | omeGs; InaCs; omeCs; InaAs; omeUs; |
| m01 | | | | | InaTs; omeAs; InaTs; omeGs; InaT-Sup |
| NR1H | NR1 | human | 61 | TAGAATTGAAGTAAC | InaTs; omeAs; InaGs; omeAs; InaAs; |
| 4-21 | H4 | | | | omeUs; InaTs; omeGs; InaAs; omeAs; |
| m01 | | | | | InaGs; omeUs; InaAs; omeAs; InaC-Sup |
| NR1H | NR1 | human | 62 | TGAAGTAACAATCAA | InaTs; omeGs; InaAs; omeAs; InaGs; |
| 4-22 | H4 | | | | omeUs; InaAs; omeAs; InaCs; omeAs; |
| m01 | | | | | InaAs; omeUs; InaCs; omeAs; InaA-Sup |
| NR1H | NR1 | human | 63 | AAGTAACAATCAATT | InaAs; omeAs; InaGs; omeUs; InaAs; |
| 4-23 | H4 | | | | omeAs; InaCs; omeAs; InaAs; omeUs; |
| m01 | | | | | InaCs; omeAs; InaAs; omeUs; InaT-Sup |
| NR1H | NR1 | human | 64 | TCATCAAGATTTCTT | InaTs; omeCs; InaAs; omeUs; InaCs; |
| 4-24 | H4 | | | | omeAs; InaAs; omeGs; InaAs; omeUs; |
| m01 | | | | | InaTs; omeUs; InaCs; omeUs; InaT-Sup |
| NR1H | NR1 | human | 65 | TATCTAGCCCAATAT | InaTs; omeAs; InaTs; omeCs; InaTs; |
| 4-25 | H4 | | | | omeAs; InaGs; omeCs; InaCs; omeCs; |
| m01 | | | | | InaAs; omeAs; InaTs; omeAs; InaT-Sup |
| NR1H | NR1 | human | 66 | TTCTATCTAGCCCAA | InaTs; omeUs; InaCs; omeUs; InaAs; |
| 4-26 | H4 | | | | omeUs; InaCs; omeUs; InaAs; omeGs; |
| m01 | | | | | InaCs; omeCs; InaCs; omeAs; InaA-Sup |
| FXN- | FXN | human | 67 | CTCCGCCCTCCAGTTT | InaCs; omeUs; omeCs; omeCs; InaGs; |
| 837 | | | | TTATTATTTTGCTTTTT | omeCs; omeCs; omeCs; InaTs; omeCs; |
| m100 | | | | | omeCs; omeAs; InaGs; omeUs; omeUs; |
| 0 | | | | | omeUs; InaTs; omeUs; omeAs; omeUs; |
| | | | | | InaTs; omeAs; omeUs; omeUs; InaTs; |
| | | | | | omeUs; omeGs; omeCs; InaTs; omeUs; |
| | | | | | omeUs; omeUs; InaT-Sup |
| FXN- | FXN | human | 68 | CCGCCCTCCAGTTTTT | InaCs; omeCs; omeGs; omeCs; InaCs; |
| 838 | | | | ATTATTTTGCTTTTT | omeCs; omeUs; omeCs; InaCs; omeAs; |
| m100 | | | | | omeGs; omeUs; InaTs; omeUs; omeUs; |
| 0 | | | | | omeUs; InaAs; omeUs; omeUs; omeAs; |
| | | | | | InaTs; omeUs; omeUs; omeUs; InaGs; |
| | | | | | omeCs; omeUs; omeUs; InaTs; omeUs; |
| | | | | | InaT-Sup |
| FXN- | FXN | human | 69 | GCCCTCCAGTTTTTAT | InaGs; omeCs; omeCs; omeCs; InaTs; |
| 839 | | | | TATTTTGCTTTTT | omeCs; omeCs; omeAs; InaGs; omeUs; |
| m100 | | | | | omeUs; omeUs; InaTs; omeUs; omeAs; |
| 0 | | | | | omeUs; InaTs; omeAs; omeUs; omeUs; |
| | | | | | InaTs; omeUs; omeGs; omeCs; InaTs; |
| | | | | | omeUs; omeUs; omeUs; InaT-Sup |
| FXN- | FXN | human | 70 | CGCTCCGCCCTCCAGT | InaCs; omeGs; omeCs; omeUs; InaCs; |
| 840 | | | | TTTTATTATTTTGCTTT | omeCs; omeGs; omeCs; InaCs; omeCs; |
| m100 | | | | | omeUs; omeCs; InaCs; omeAs; omeGs; |
| 0 | | | | | omeUs; InaTs; omeUs; omeUs; omeUs; |
| | | | | | InaAs; omeUs; omeUs; omeAs; InaTs; |
| | | | | | omeUs; omeUs; omeUs; InaGs; omeCs; |
| | | | | | omeUs; omeUs; InaT-Sup |
| FXN- | FXN | human | 71 | CGCTCCGCCCTCCAGT | InaCs; omeGs; omeCs; omeUs; InaCs; |
| 841 | | | | TTTTATTATTTTGCT | omeCs; omeGs; omeCs; InaCs; omeCs; |
| m100 | | | | | omeUs; omeCs; InaCs; omeAs; omeGs; |
| 0 | | | | | omeUs; InaTs; omeUs; omeUs; omeUs; |
| | | | | | InaAs; omeUs; omeUs; omeAs; InaTs; |
| | | | | | omeUs; omeUs; omeUs; InaGs; omeCs; |
| | | | | | InaT-Sup |
| FXN- | FXN | human | 72 | CGCTCCGCCCTCCAGT | InaCs; omeGs; omeCs; omeUs; InaCs; |
| 842 | | | | TTTTATTATTTTG | omeCs; omeGs; omeCs; InaCs; omeCs; |
| m100 | | | | | omeUs; omeCs; InaCs; omeAs; omeGs; |
| 0 | | | | | omeUs; InaTs; omeUs; omeUs; omeUs; |
| | | | | | InaAs; omeUs; omeUs; omeAs; InaTs; |
| | | | | | omeUs; omeUs; omeUs; InaG-Sup |
| FXN- | FXN | human | 73 | CTCCGCCCTCCAGTTT | InaCs; omeUs; omeCs; omeCs; InaGs; |
| 843 | | | | TTATTATTTTGCTTT | omeCs; omeCs; omeCs; InaTs; omeCs; |
| m100 | | | | | omeCs; omeAs; InaGs; omeUs; omeUs; |
| 0 | | | | | omeUs; InaTs; omeUs; omeAs; omeUs; |
| | | | | | InaTs; omeAs; omeUs; omeUs; InaTs; |
| | | | | | omeUs; omeGs; omeCs; InaTs; omeUs; |
| | | | | | InaT-Sup |
| FXN- | FXN | human | 74 | CCGCCCTCCAGTTTTT | InaCs; omeCs; omeGs; InaCs; omeCs; |
| 844 | | | | ATTATTTTGCT | omeCs; InaTs; omeCs; omeCs; InaAs; |
| m100 | | | | | omeGs; omeUs; InaTs; omeUs; omeUs; |
| 0 | | | | | InaTs; omeAs; omeUs; InaTs; omeAs; |
| | | | | | omeUs; InaTs; omeUs; omeUs; InaGs; |
| | | | | | omeCs; InaT-Sup |
| FXN- | FXN | human | 75 | GCCCTCCAGTTTTTAT | InaGs; omeCs; omeCs; InaCs; omeUs; |
| 845 | | | | TATTTTGCT | omeCs; InaCs; omeAs; omeGs; InaTs; |
| m100 | | | | | omeUs; omeUs; InaTs; omeUs; omeAs; |
| 0 | | | | | InaTs; omeUs; omeAs; InaTs; omeUs; |
| | | | | | omeUs; InaTs; omeGs; omeCs; InaT-Sup |
| FXN- | FXN | human | 76 | CCCTCCAGTTTTTATT | InaCs; omeCs; omeCs; InaTs; omeCs; |
| 846 | | | | ATTTTGC | omeCs; InaAs; omeGs; omeUs; InaTs; |
| m100 | | | | | omeUs; omeUs; InaTs; omeAs; omeUs; |
| 0 | | | | | InaTs; omeAs; omeUs; InaTs; omeUs; |
| | | | | | omeUs; InaGs; InaC-Sup |
| FXN- | FXN | human | 77 | CCTCCAGTTTTTATTAT | InaCs; omeCs; omeUs; InaCs; omeCs; |
| 847 | | | | TTTG | omeAs; InaGs; omeUs; omeUs; InaTs; |
| m100 | | | | | omeUs; omeUs; InaAs; omeUs; omeUs; |
| 0 | | | | | InaAs; omeUs; omeUs; InaTs; omeUs; |
| | | | | | InaG-Sup |
| FXN- | FXN | human | 78 | GCTCCGCCCTCCAGAT | InaGs; omeCs; omeUs; omeCs; InaCs; |
| 848 | | | | TATTTTGCTTTTT | omeGs; omeCs; omeCs; InaCs; omeUs; |
| m100 | | | | | omeCs; omeCs; InaAs; omeGs; omeAs; |
| 0 | | | | | omeUs; InaTs; omeAs; omeUs; omeUs; |
| | | | | | InaTs; omeUs; omeGs; omeCs; InaTs; |
| | | | | | omeUs; omeUs; omeUs; InaT-Sup |
| FXN- | FXN | human | 79 | TCCGCCCTCCAGATTA | InaTs; omeCs; omeCs; InaGs; omeCs; |
| 849 | | | | TTTTGCTTTTT | omeCs; InaCs; omeUs; omeCs; InaCs; |
| m100 | | | | | omeAs; omeGs; InaAs; omeUs; omeUs; |
| 0 | | | | | InaAs; omeUs; omeUs; InaTs; omeUs; |
| | | | | | omeGs; InaCs; omeUs; omeUs; InaTs; |
| | | | | | omeUs; InaT-Sup |
| FXN- | FXN | human | 80 | CGCCCTCCAGATTATT | InaCs; omeGs; omeCs; InaCs; omeCs; |
| 850 | | | | TTGCTTTTT | omeUs; InaCs; omeCs; omeAs; InaGs; |
| m100 | | | | | omeAs; omeUs; InaTs; omeAs; omeUs; |
| 0 | | | | | InaTs; omeUs; omeUs; InaGs; omeCs; |
| | | | | | omeUs; InaTs; omeUs; omeUs; InaT-Sup |
| FXN- | FXN | human | 81 | CCCTCCAGATTATTTT | InaCs; omeCs; omeCs; InaTs; omeCs; |
| 851 | | | | GCTTTTT | omeCs; InaAs; omeGs; omeAs; InaTs; |
| m100 | | | | | omeUs; omeAs; InaTs; omeUs; omeUs; |
| 0 | | | | | InaTs; omeGs; omeCs; InaTs; omeUs; |
| | | | | | omeUs; InaTs; InaT-Sup |
| FXN- | FXN | human | 82 | GCTCCGCCCTCCAGAT | InaGs; omeCs; omeUs; InaCs; omeCs; |
| 852 | | | | TATTTTGCTTT | omeGs; InaCs; omeCs; omeCs; InaTs; |
| m100 | | | | | omeCs; omeCs; InaAs; omeGs; omeAs; |
| 0 | | | | | InaTs; omeUs; omeAs; InaTs; omeUs; |
| | | | | | omeUs; InaTs; omeGs; omeCs; InaTs; |
| | | | | | omeUs; InaT-Sup |
| FXN- | FXN | human | 83 | GCTCCGCCCTCCAGAT | InaGs; omeCs; omeUs; InaCs; omeCs; |
| 853 | | | | TATTTTGCT | omeGs; InaCs; omeCs; omeCs; InaTs; |
| m100 | | | | | omeCs; omeCs; InaAs; omeGs; omeAs; |
| 0 | | | | | InaTs; omeUs; omeAs; InaTs; omeUs; |
| | | | | | omeUs; InaTs; omeGs; omeCs; InaT-Sup |
| FXN- | FXN | human | 84 | GCTCCGCCCTCCAGAT | InaGs; omeCs; omeUs; InaCs; omeCs; |
| 854 | | | | TATTTTG | omeGs; InaCs; omeCs; omeCs; InaTs; |
| m100 | | | | | omeCs; omeCs; InaAs; omeGs; omeAs; |
| 0 | | | | | InaTs; omeUs; omeAs; InaTs; omeUs; |
| | | | | | omeUs; InaTs; InaG-Sup |
| FXN- | FXN | human | 85 | TCCGCCCTCCAGATTA | InaTs; omeCs; omeCs; InaGs; omeCs; |
| 855 | | | | TTTTGCTTT | omeCs; InaCs; omeUs; omeCs; InaCs; |
| m100 | | | | | omeAs; omeGs; InaAs; omeUs; omeUs; |
| 0 | | | | | InaAs; omeUs; omeUs; InaTs; omeUs; |
| | | | | | omeGs; InaCs; omeUs; omeUs; InaT-Sup |
| FXN- | FXN | human | 86 | CGCCCTCCAGATTATT | InaCs; omeGs; omeCs; InaCs; omeCs; |
| 856 | | | | TTGCT | omeUs; InaCs; omeCs; omeAs; InaGs; |
| m100 | | | | | omeAs; omeUs; InaTs; omeAs; omeUs; |
| 0 | | | | | InaTs; omeUs; omeUs; InaGs; omeCs; |
| | | | | | InaT-Sup |
| FXN- | FXN | human | 87 | GCCCTCCAGATTATTT | InaGs; omeCs; InaCs; omeCs; InaTs; |
| 857 | | | | TGC | omeCs; InaCs; omeAs; InaGs; omeAs; |
| m01 | | | | | InaTs; omeUs; InaAs; omeUs; InaTs; |
| | | | | | omeUs; InaTs; omeGs; InaC-Sup |
| FXN- | FXN | human | 88 | CCCTCCAGATTATTTT | InaCs; omeCs; InaCs; omeUs; InaCs; |
| 858 | | | | G | omeCs; InaAs; omeGs; InaAs; omeUs; |
| m01 | | | | | InaTs; omeAs; InaTs; omeUs; InaTs; |
| | | | | | omeUs; InaG-Sup |
| FXN- | FXN | human | 89 | CTCCAGATTATTTTG | InaCs; omeUs; InaCs; omeCs; InaAs; |
| 859 | | | | | omeGs; InaAs; omeUs; InaTs; omeAs; |
| m01 | | | | | InaTs; omeUs; InaTs; omeUs; InaG-Sup |
| FXN- | FXN | human | 90 | CGCTCCGCCCTCCAGT | dCs; InaGs; dCs; InaTs; dCs; InaCs; |
| 461 | | | | TTTTATTATTTTGCTTT | dGs; InaCs; dCs; InaCs; dTs; InaCs; |
| m02 | | | | TT | dCs; InaAs; dGs;InaTs; dTs; InaTs; |
| | | | | | dTs; InaTs; dAs; InaTs; dTs; InaAs; |
| | | | | | dTs; InaTs; dTs; InaTs; dGs; InaCs; |
| | | | | | dTs; InaTs; dTs; InaTs; dT-Sup |
| Apoa1 | APO | mouse | 91 | AGTTCAAGGATCAGC | InaAs; dGs; dTs; InaTs; dCs; dAs; |
| _mus- | A1 | | | CATTTTGGAAAGG | InaAs; dGs; dGs; InaAs; dTs; dCs; |
| 77 | | | | | InaAs; dGs; dCs; InaCs; dAs; dTs; |
| m100 | | | | | InaTs; dTs; dTs; InaGs; dGs; dAs; |
| 0 | | | | | InaAs; dAs; dGs; InaG-Sup |
| Apoa1 | APO | mouse | 92 | TCAAGGATCAGCCATT | InaTs; dCs; dAs; InaAs; dGs; dGs; |
| _mus- | A1 | | | TTGGAAAGG | InaAs; dTs; dCs; InaAs; dGs; dCs; |
| 78 | | | | | InaCs; dAs; dTs; InaTs; dTs; dTs; |
| m100 | | | | | InaGs; dGs; dAs; InaAs; dAs; dGs; |
| 0 | | | | | InaG-Sup |
| Apoa1 | APO | mouse | 93 | AAGGATCAGCCATTTT | InaAs; dAs; dGs; InaGs; dAs; dTs; |
| _mus- | A1 | | | GGAAAGG | InaCs; dAs; dGs; InaCs; dCs; dAs; |
| 79 | | | | | InaTs; dTs; dTs; InaTs; dGs; dGs; |
| m100 | | | | | InaAs; dAs; dAs; InaGs; InaG-Sup |
| 0 | | | | | |
| Apoa1 | APO | mouse | 94 | GGATCAGCCATTTTG | InaGs; dGs; dAs; InaTs; dCs; dAs; |
| _mus- | A1 | | | GAAAGG | InaGs; dCs; dCs; InaAs; dTs; dTs; |
| 80 | | | | | InaTs; dTs; dGs; InaGs; dAs; dAs; |
| m100 | | | | | InaAs; dGs; InaG-Sup |
| 0 | | | | | |
| Apoa1 | APO | mouse | 95 | AGTTCAAGGATCAGC | InaAs; dGs; dTs; InaTs; dCs; dAs; |
| _mus- | A1 | | | CATTTTGGAA | InaAs; dGs; dGs; InaAs; dTs; dCs; |
| 81 | | | | | InaAs; dGs; dCs; InaCs; dAs; dTs; |
| m100 | | | | | InaTs; dTs; dTs; InaGs; dGs; dAs; |
| 0 | | | | | InaA-Sup |
| Apoa1 | APO | mouse | 96 | AGTTCAAGGATCAGC | InaAs; dGs; dTs; InaTs; dCs; dAs; |
| _mus- | A1 | | | CATTTTGG | InaAs; dGs; dGs; InaAs; dTs; dCs; |
| 82 | | | | | InaAs; dGs; dCs; InaCs; dAs; dTs; |
| m100 | | | | | InaTs; dTs; dTs; InaGs; InaG-Sup |
| 0 | | | | | |
| Apoa1 | APO | mouse | 97 | GTTCAAGGATCAGCC | InaGs; dTs; dTs; InaCs; dAs; dAs; |
| _mus- | A2 | | | ATTTTGGAAAGG | InaGs; dGs; dAs; InaTs; dCs; dAs; |
| 83 | | | | | InaGs; dCs; dCs; InaAs; dTs; dTs; |
| m100 | | | | | InaTs; dTs; dGs; InaGs; dAs; dAs; |
| 0 | | | | | InaAs; dGs; InaG-Sup |
| Apoa1 | APO | mouse | 98 | TCAAGGATCAGCCATT | InaTs; dCs; dAs; InaAs; dGs; dGs; |
| _mus- | A1 | | | TTGGAAA | InaAs; dTs; dCs; InaAs; dGs; dCs; |
| 84 | | | | | InaCs; dAs; dTs; InaTs; dTs; dTs; |
| m100 | | | | | InaGs; dGs; dAs; InaAs; InaA-Sup |
| 0 | | | | | |
| Apoa1 | APO | mouse | 99 | AAGGATCAGCCATTTT | InaAs; dAs; dGs; InaGs; dAs; dTs; |
| _mus- | A1 | | | GGAAA | InaCs; dAs; dGs; InaCs; dCs; dAs; |
| 85 | | | | | InaTs; dTs; dTs; InaTs; dGs; dGs; |
| m100 | | | | | InaAs; dAs; InaA-Sup |
| 0 | | | | | |
| Apoa1 | APO | mouse | 100 | AGGATCAGCCATTTTG | InaAs; dGs; InaGs; dAs; InaTs;dCs; |
| _mus- | A1 | | | GAA | InaAs; dGs; InaCs; dCs; InaAs; dTs; |
| 86 | | | | | InaTs; dTs; InaTs; dGs; InaGs; dAs; |
| m12 | | | | | InaA-Sup |
| Apoa1 | APO | mouse | 101 | GGATCAGCCATTTTG | InaGs; dGs; InaAs; dTs; InaCs; dAs; |
| _mus- | A1 | | | GA | InaGs; dCs; InaCs; dAs; InaTs; dTs; |
| 87 | | | | | InaTs; dTs; InaGs; dGs; InaA-Sup |
| m12 | | | | | |
| Apoa1 | APO | mouse | 102 | CTCCGACAGTCTGCCA | InaCs; dTs; dCs; InaCs; dGs; dAs; |
| _mus- | A1 | | | TTTTGGAAAGGT | InaCs; dAs; dGs; InaTs; dCs; dTs; |
| 88 | | | | | InaGs; dCs; dCs; InaAs; dTs; dTs; |
| m100 | | | | | InaTs; dTs; dGs; InaGs; dAs; dAs; |
| 0 | | | | | InaAs; dGs; dGs; InaT-Sup |
| Apoa1 | APO | mouse | 103 | CGACAGTCTGCCATTT | InaCs; dGs; dAs; InaCs; dAs; dGs; |
| _mus- | A1 | | | TGGAAAGGT | InaTs; dCs; dTs; InaGs; dCs; dCs; |
| 89 | | | | | InaAs; dTs; dTs; InaTs; dTs; dGs; |
| m100 | | | | | InaGs; dAs; dAs; InaAs; dGs; dGs; |
| 0 | | | | | InaT-Sup |
| Apoa1 | APO | mouse | 104 | ACAGTCTGCCATTTTG | InaAs; dCs; dAs; InaGs; dTs; dCs; |
| _mus- | A1 | | | GAAAGGT | InaTs; dGs; dCs; InaCs; dAs; dTs; |
| 90 | | | | | InaTs; dTs; dTs; InaGs; dGs; dAs; |
| m100 | | | | | InaAs; dAs; dGs; InaGs; InaT-Sup |
| 0 | | | | | |
| Apoa1 | APO | mouse | 105 | CTCCGACAGTCTGCCA | InaCs; dTs; dCs; InaCs; dGs; dAs; |
| _mus- | A1 | | | TTTTGGAAA | InaCs; dAs; dGs; InaTs; dCs; dTs; |
| 91 | | | | | InaGs; dCs; dCs; InaAs; dTs; dTs; |
| m100 | | | | | InaTs; dTs; dGs; InaGs; dAs; dAs; |
| 0 | | | | | InaA-Sup |
| Apoa1 | APO | mouse | 106 | CTCCGACAGTCTGCCA | InaCs; dTs; dCs; InaCs; dGs; dAs; |
| _mus- | A1 | | | TTTTGGA | InaCs; dAs; dGs; InaTs; dCs; dTs; |
| 92 | | | | | InaGs; dCs; dCs; InaAs; dTs; dTs; |
| m100 | | | | | InaTs; dTs; dGs; InaGs; InaA-Sup |
| 0 | | | | | |
| Apoa1 | APO | mouse | 107 | CTCCGACAGTCTGCCA | InaCs; dTs; dCs; InaCs; dGs; dAs; |
| _mus- | A1 | | | TTTTG | InaCs; dAs; dGs; InaTs; dCs; dTs; |
| 93 | | | | | InaGs; dCs; dCs; InaAs; dTs; dTs; |
| m100 | | | | | InaTs; dTs; InaG-Sup |
| 0 | | | | | |
| Apoa1 | APO | mouse | 108 | CTCCGACAGTCTGCCA | InaCs; dTs; dCs; InaCs; dGs; dAs; |
| _mus- | A1 | | | TTTTGGAAAGG | InaCs; dAs; dGs; InaTs; dCs; dTs; |
| 94 | | | | | InaGs; dCs; dCs; InaAs; dTs; dTs; |
| m100 | | | | | InaTs; dTs; dGs; InaGs; dAs; dAs; |
| 0 | | | | | InaAs; dGs; InaG-Sup |
| Apoa1 | APO | mouse | 109 | CCGACAGTCTGCCATT | InaCs; dCs; dGs; InaAs; dCs; dAs; |
| _mus- | A1 | | | TTGGAAA | InaGs; dTs; dCs; InaTs; dGs; dCs; |
| 95 | | | | | InaCs; dAs; dTs; InaTs; dTs; dTs; |
| m100 | | | | | InaGs; dGs; dAs; InaAs; InaA-Sup |
| 0 | | | | | |
| Apoa1 | APO | mouse | 110 | GACAGTCTGCCATTTT | InaGs; dAs; InaCs; dAs; InaGs; dTs; |
| _mus- | A1 | | | GGA | InaCs; dTs; InaGs; dCs; InaCs; dAs; |
| 96 | | | | | InaTs; dTs; InaTs; dTs; InaGs; dGs; |
| m12 | | | | | InaA-Sup |
| Apoa1 | APO | mouse | 111 | ACAGTCTGCCATTTTG | InaAs; dCs; InaAs; dGs; InaTs; dCs; |
| _mus- | A1 | | | G | InaTs; dGs; InaCs; dCs; InaAs; dTs; |
| 97 | | | | | InaTs; dTs; InaTs; dGs; InaG-Sup |
| m12 | | | | | |
| Apoa1 | APO | mouse | 112 | CGGAGCTCTCCGACA | InaCs; dGs; dGs; InaAs; dGs; dCs; |
| _mus- | A1 | | | CATTTTGGAAAGGTT | InaTs; dCs; dTs; InaCs; dCs; dGs; |
| 98 | | | | | InaAs; dCs; dAs; InaCs; dAs; dTs; |
| m100 | | | | | InaTs; dTs; dTs; InaGs; dGs; dAs; |
| 0 | | | | | InaAs; dAs; dGs; InaGs; dTs; InaT- |
| | | | | | Sup |
| Apoa1 | APO | mouse | 113 | GGAGCTCTCCGACAC | InaGs; dGs; dAs; InaGs; dCs; dTs; |
| _mus- | A1 | | | ATTTTGGAAAGGTT | InaCs; dTs; dCs; InaCs; dGs; dAs; |
| 99 | | | | | InaCs; dAs; dCs; InaAs; dTs; dTs; |
| m100 | | | | | InaTs; dTs; dGs; InaGs; dAs; dAs; |
| 0 | | | | | InaAs; dGs; dGs; InaTs; InaT-Sup |
| Apoa1 | APO | mouse | 114 | AGCTCTCCGACACATT | InaAs; dGs; dCs; InaTs; dCs; dTs; |
| _mus- | A1 | | | TTGGAAAGG | InaCs; dCs; dGs; InaAs; dCs; dAs; |
| 100 | | | | | InaCs; dAs; dTs; InaTs; dTs; dTs; |
| m100 | | | | | InaGs; dGs; dAs; InaAs; dAs; dGs; |
| 0 | | | | | InaG-Sup |
| Apoa1 | APO | mouse | 115 | CTCTCCGACACATTTT | InaCs; dTs; dCs; InaTs; dCs; dCs; |
| _mus- | A1 | | | GGAAA | InaGs; dAs; dCs; InaAs; dCs; dAs; |
| 101 | | | | | InaTs; dTs; dTs; InaTs; dGs; dGs; |
| m100 | | | | | InaAs; dAs; InaA-Sup |
| 0 | | | | | |
| Apoa1 | APO | mouse | 116 | TCTCCGACACATTTTG | InaTs; dCs; InaTs; dCs; InaCs; dGs; |
| _mus- | A1 | | | GAA | InaAs; dCs; InaAs; dCs; InaAs; dTs; |
| 102 | | | | | InaTs; dTs; InaTs; dGs; InaGs; dAs; |
| m12 | | | | | InaA-Sup |
| Apoa1 | APO | mouse | 117 | CTCCGACACATTTTGG | InaCs; dTs; InaCs; dCs; InaGs; dAs; |
| _mus- | A1 | | | A | InaCs; dAs; InaCs; dAs; InaTs; dTs; |
| 103 | | | | | InaTs; dTs; InaGs; dGs; InaA-Sup |
| m12 | | | | | |
| Apoa1 | APO | mouse | 118 | TCCGACACATTTTGG | InaTs; dCs; InaCs; dGs; InaAs; dCs; |
| _mus- | A1 | | | | InaAs; dCs; InaAs; dTs; InaTs; dTs; |
| 104 | | | | | InaTs; dGs; InaG-Sup |
| m12 | | | | | |
| Apoa1 | APO | mouse | 124 | AGTTCAAGGATCAGC | InaAs; dGs; InaTs; dTs; InamCs; dAs; |
| _mus- | A1 | | | | InaAs; dGs; InaGs; dAs; InaTs; dCs; |
| 07 | | | | | InaAs; dGs; InamC-Sup |
| m12 | | | | | |
| Apoa1 | APO | mouse | 124 | AGTTCAAGGATCAGC | InaAs; omeGs; InaTs; omeUs; InamCs; |
| _mus- | A1 | | | | omeAs; InaAs; omeGs; InaGs; InaAs; |
| 07 | | | | | InaTs; InamCs; InaAs; InaGs; InamC- |
| m100 | | | | | Sup |
| 0 opt1 | | | | | |
| v23 | | | | | |
| Apoa1 | APO | mouse | 125 | CATTTTGGAAAGG | InamCs; InaAs; InaTs; InaTs; InaTs; |
| _mus- | A1 | | | | dTs; InaGs; dGs; InaAs; dAs; InaAs; |
| 20 | | | | | InaGs; InaG-Sup |
| m100 | | | | | |
| 0 opt1 | | | | | |
| v3 | | | | | |

TABLE 4
Oligonucleotide modifications

| Symbol | Feature Description |
|---|---|
| bio | 5′ biotin |
| dAs | DNA w/3′ thiophosphate |
| dCs | DNA w/3′ thiophosphate |
| dGs | DNA w/3′ thiophosphate |
| dTs | DNA w/3′ thiophosphate |
| dA | DNA |
| dC | DNA |
| dG | DNA |
| dT | DNA |
| enaAs | ENA w/3′ thiophosphate |
| enaCs | ENA w/3′ thiophosphate |
| enaGs | ENA w/3′ thiophosphate |
| enaTs | ENA w/3′ thiophosphate |
| fluAs | 2′-fluoro w/3′ thiophosphate |
| fluCs | 2′-fluoro w/3′ thiophosphate |
| fluGs | 2′-fluoro w/3′ thiophosphate |
| fluUs | 2′-fluoro w/3′ thiophosphate |
| lnaAs | LNA w/3′ thiophosphate |
| lnaCs | LNA w/3′ thiophosphate |
| lnaGs | LNA w/3′ thiophosphate |
| lnaTs | LNA w/3′ thiophosphate |
| omeAs | 2′-OMe w/3′ thiophosphate |
| omeCs | 2′-OMe w/3′ thiophosphate |
| omeGs | 2′-OMe w/3′ thiophosphate |
| omeTs | 2′-OMe w/3′ thiophosphate |
| lnaAs-Sup | LNA w/3′ thiophosphate at 3′ terminus |
| lnaCs-Sup | LNA w/3′ thiophosphate at 3′ terminus |
| lnaGs-Sup | LNA w/3′ thiophosphate at 3′ terminus |
| lnaTs-Sup | LNA w/3′ thiophosphate at 3′ terminus |
| lnaA-Sup | LNA w/3′ OH at 3′ terminus |
| lnaC-Sup | LNA w/3′ OH at 3′ terminus |
| lnaG-Sup | LNA w/3′ OH at 3′ terminus |
| lnaT-Sup | LNA w/3′ OH at 3′ terminus |
| omeA-Sup | 2′-OMe w/3′ OH at 3′ terminus |
| omeC-Sup | 2′-OMe w/3′ OH at 3′ terminus |
| omeG-Sup | 2′-OMe w/3′ OH at 3′ terminus |
| omeU-Sup | 2′-OMe w/3′ OH at 3′ terminus |
| dAs-Sup | DNA w/3′ thiophosphate at 3′ terminus |
| dCs-Sup | DNA w/3′ thiophosphate at 3′ terminus |
| dGs-Sup | DNA w/3′ thiophosphate at 3′ terminus |
| dTs-Sup | DNA w/3′ thiophosphate at 3′ terminus |
| dA-Sup | DNA w/3′ OH at 3′ terminus |
| dC-Sup | DNA w/3′ OH at 3′ terminus |
| dG-Sup | DNA w/3′ OH at 3′ terminus |
| dT-Sup | DNA w/3′ OH at 3′ terminus |

The suffix “Sup” in Table 4 indicates that a 3′ end nucleotide may, for synthesis purposes, be conjugated to a solid support. It should be appreciated that in general when conjugated to a solid support for synthesis, the synthesized oligonucleotide is released such that the solid support is not part of the final oligonucleotide product. Note than a “m” appearing before a C nucleotide (e.g., dmCs, lnamCs) indicates a 5-methylcytosine nucleotide.

### APOA1

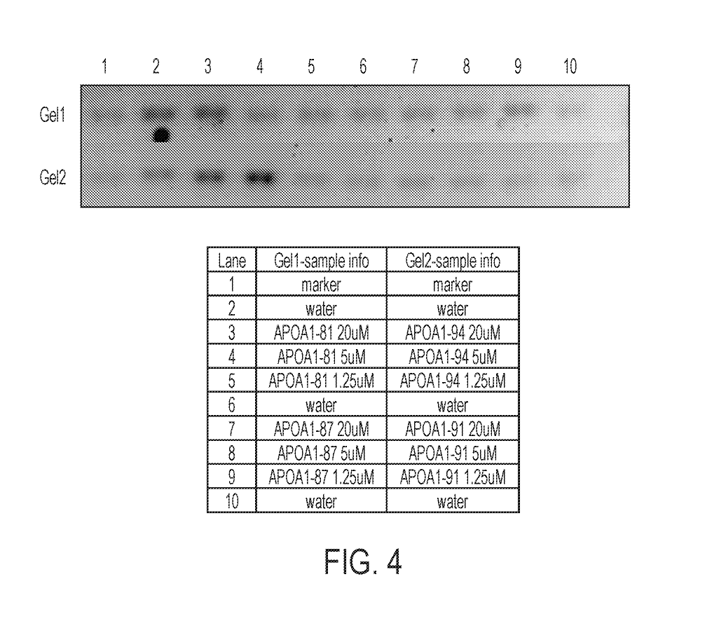

Mouse APOA1 pseudo-circularization oligos were screened in primary mouse hepatocytes. Oligos were delivered gymnotically at 20, 5 and 1.25 micromolar doses. Measurements were taken at day 2. FIG. 4 shows Western blots of proteins from culture media. The “water” lanes show control cases without oligos (only water was added to wells in volumes equal to oligo treatment volume). Abcam ab20453 was used as APOA1 antibody.

APOA1-81 oligo (Apoa1_mus-81 m1000) induced APOA1 protein upregulation in a dose responsive manner (FIG. 4). APOA1-94 (Apoa1_mus-81 m1000) showed robust APOA1 protein upregulation at low doses. The level of upregulation was lower at higher doses. Without wishing to be bound by theory, this lowering may be caused may one or more factors, including kinetics, potency of oligo action, and/or duration dependence of treatment.

### FXN

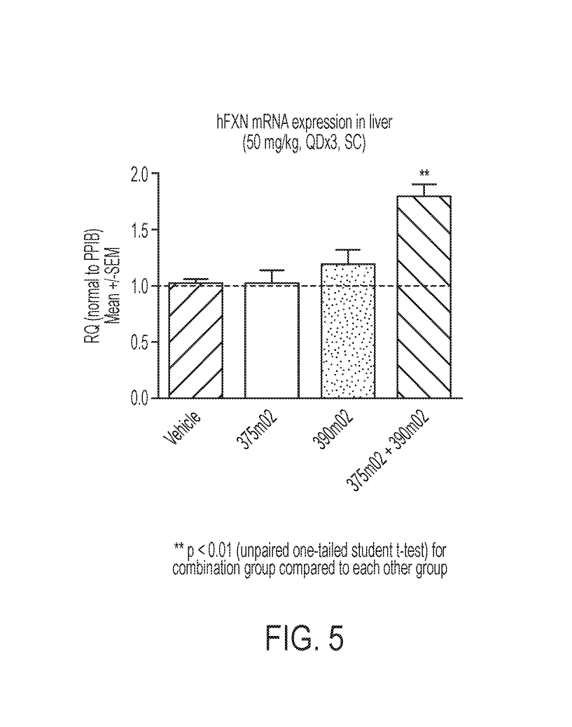

Exemplary human FXN oligos 375 (5′) and 390 (3′) upregulated human frataxin mRNA in the liver (FIG. 5). These oligo sequences are provided in Table 5 below. The oligos were injected subcutaneously alone or in combination to the Sarsero FRDA mouse model. Vehicle (PBS) was injected as control. Treatment dose was 50 mg/kg/day per oligo at day 0, 1, 2. The collection was done 2 days post last dose. All treatments were tolerated. The human FXN livers of this model were measured with QPCR and normalized to the PBS group. Each treatment group had 6 mice (n=6). Vehicle group has 28 animals.

TABLE 5
Other exemplary FXN oligos

| | | | SEQ | | |
|---|---|---|---|---|---|
| Oligo | | | ID | | |
| Name | Gene | Organism | NO. | Base Sequence | Formatted Sequence |
| FXN- | FXN | human | 119 | CGCTCCGCCCTCCAG | dCs; InaGs; dCs; InaTs; dCs; |
| 375 | | | | | InaCs; dGs; InaCs; dCs; InaCs; |
| | | | | | dTs; InaCs; dCs; InaAs; dG-Sup |
| FXN- | FXN | human | 120 | ATTATTTTGCTTTTT | dAs; InaTs; dTs; InaAs; dTs; |
| 390 | | | | | InaTs; dTs; InaTs; dGs; InaCs; |
| | | | | | dTs; InaTs; dTs; InaTs; dT-Sup |

These results show that oligos targeting 3′ and 5′ ends of RNAs, as well as pseudocircularization oligos, can upregulate gene expression.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A method of increasing THRB or NR1H4 gene expression in a cell, the method comprising:
delivering to a cell an oligonucleotide comprising the general formula 5′-X₁-X₂-3′, wherein X₁ comprises 5 to 20 nucleotides that have a region of complementarity that is complementary with at least 5 contiguous nucleotides of an RNA transcript encoded by the THRB or NR1H4 gene, wherein the nucleotide at the 3′-end of the region of complementary of X₁ is complementary with the nucleotide at the transcription start site of the RNA transcript; and X₂ comprises 1 to 20 nucleotides.

2. The method of claim 1, wherein the RNA transcript has a 7-methylguanosine cap at its 5′-end.

3. The method of claim 1, wherein the RNA transcript has a 7-methylguanosine cap, and wherein the nucleotide at the 3′-end of the region of complementary of X₁ is complementary with the nucleotide of the RNA transcript that is immediately internal to the 7-methylguanosine cap.

4. The method of claim 1, wherein at least the first nucleotide at the 5′-end of X₂ is a pyrimidine complementary with guanine.

5. The method of claim 2, wherein the second nucleotide at the 5′-end of X₂ is a pyrimidine complementary with guanine.

6. The method of claim 1, wherein X₂ comprises the formula 5′-Y₁-Y₂-Y₃-3′, wherein X₂ forms a stem-loop structure having a loop region comprising the nucleotides of Y₂ and a stem region comprising at least two contiguous nucleotides of Y₁ hybridized with at least two contiguous nucleotides of Y₃.

7. The method of claim 6, wherein Y₁, Y₂ and Y₃ independently comprise 1 to 10 nucleotides.

8. The method of claim 6 or 7, wherein Y₃ comprises, at a position immediately following the 3′-end of the stem region, a pyrimidine complementary with guanine.

9. The method of any one of claims 2 to 8, wherein the pyrimidine complementary with guanine is cytosine.

10. The method of claim 1, wherein X₂ comprises a region of complementarity that is complementary with at least 5 contiguous nucleotides of the RNA transcript that do not overlap the region of the RNA transcript that is complementary with the region of complementarity of X₁.

11. The method of claim 10, wherein the region of complementarity of X₂ is within 100 nucleotides of a polyadenylation junction of the RNA transcript.

12. The method of claim 11, wherein the region of complementarity of X₂ is complementary with the RNA transcript immediately adjacent to or overlapping the polyadenylation junction of the RNA transcript.

13. The method of claim 11 or 12, wherein X₂ further comprises at least 2 consecutive pyrimidine nucleotides complementary with adenine nucleotides of the poly(A) tail of the RNA transcript.

14. The method of any one of claims 1 to 13, wherein the RNA transcript is an mRNA, non-coding RNA, long non-coding RNA, miRNA, or snoRNA or any other suitable RNA.

15. The method of any one of claims 1 to 14, wherein the RNA transcript is an mRNA transcript, and wherein X₂ comprises a region of complementarity that is complementary with at least 5 contiguous nucleotides in the 3′-UTR of the transcript.

16. The method of any one of claims 1 to 15, wherein the RNA transcript is an mRNA and the delivery results in an increase in the level of a protein encoded by the mRNA.

17. The method of any one of claim 16, wherein the increase in the level of the protein encoded by the mRNA is at least a 50% increase compared with an appropriate control cell to which the oligonucleotide was not delivered.

18. The method of any one of claims 1 to 15, wherein the RNA transcript is an mRNA transcript of THRB or NR1H4 as described in Table 1.

19. The method of claim 1, wherein X₂ comprises the sequence CC.

20. A method of increasing THRB or NR1H4 gene expression in a cell, the method comprising delivering to a cell an oligonucleotide of 10 to 50 nucleotides in length having a first region complementary with at least 5 consecutive nucleotides of the 5′-UTR of an mRNA transcript encoded by the THRB or NR1H4 gene, and a second region complementary with at least 5 consecutive nucleotides of the 3′-UTR, poly(A) tail, or overlapping the polyadenylation junction of the mRNA transcript.

21. The method of claim 20, wherein the first of the at least 5 consecutive nucleotides of the 5′-UTR is within 10 nucleotides of the 5′-methylguanosine cap of the mRNA transcript.

22. The method of claim 20 or 21, wherein the second region is complementary with at least 5 consecutive nucleotides overlapping the polyadenylation junction.

23. The method of any one of claims 20 to 22, further comprising 2-20 nucleotides that link the 5′ end of the first region with the 3′ end of the second region.

24. The method of any one of claims 20 to 22, further comprising 2-20 nucleotides that link the 3′ end of the first region with the 5′ end of the second region.

25. The method of any one of claims 20 to 24, wherein the oligonucleotide is 10 to 50 nucleotide in length.

26. The method of any one of claims 20 to 24, wherein the oligonucleotide is 9 to 20 nucleotide in length.

27. The method of any one of claims 20 to 26, wherein the mRNA transcript is an mRNA transcript of THRB or NR1H4 as described in Table 1.

28. A method of increasing THRB or NR1H4 gene expression in a cell, the method comprising delivering to a cell an oligonucleotide comprising the general formula 5′-X₁-X₂-3′, wherein X₁ comprises 2 to 20 pyrimidine nucleotides that form base pairs with adenine; and X₂ comprises a region of complementarity that is complementary with at least 3 contiguous nucleotides of a poly-adenylated RNA transcript encoded by the THRB or NR1H4 gene, wherein the nucleotide at the 5′-end of the region of complementary of X₂ is complementary with the nucleotide of the RNA transcript that is immediately internal to the poly-adenylation junction of the RNA transcript.

29. The method of claim 28, wherein X₁ comprises 2 to 20 thymidines or uridines.

30. The method of claim 28 or 29, wherein the poly-adenylated RNA transcript is an mRNA transcript.

31. The method of any one of claims 1 to 30, wherein the oligonucleotide comprises at least one modified internucleoside linkage.

32. The method of any one of claims 1 to 31, wherein the oligonucleotide comprises at least one modified nucleotide.

33. The method of any one of claims 1 to 32, wherein at least one nucleotide comprises a 2′ O-methyl.

34. The method of any one of claims 1 to 33, wherein the oligonucleotide comprises at least one ribonucleotide, at least one deoxyribonucleotide, at least one 2′-fluoro-deoxyribonucleotides or at least one bridged nucleotide.

35. The method of claim 34, wherein the bridged nucleotide is a LNA nucleotide, a cEt nucleotide or a ENA modified nucleotide.

36. The method of any one of claims 1 to 35, wherein each nucleotide of the oligonucleotide is a LNA nucleotide.

37. The method of any one of claims 1 to 36, wherein the nucleotides of the oligonucleotide comprise alternating deoxyribonucleotides and 2′-fluoro-deoxyribonucleotides, 2′-O-methyl nucleotides, or bridged nucleotides.

38. The method of any one of claims 1 to 37, wherein the oligonucleotide is mixmer.

39. The method of any one of claims 1 to 38, wherein the oligonucleotide is morpholino.

40. The method of any one of claims 1 to 39, wherein the cell is in vitro.

41. The method of any one of claims 1 to 39, wherein the cell is in vivo.

42. A method of increasing THRB or NR1H4 gene expression in a cell, the method comprising delivering to a cell, expressing an RNA transcript of the THRB or NR1H4 gene, an oligonucleotide of 8 to 50 nucleotides in length, the oligonucleotide comprising a region of complementarity that is complementary with at least 5 contiguous nucleotides of an RNA transcript, wherein the nucleotide at the 3′-end of the region of complementary is complementary with a nucleotide within 10 nucleotides of the transcription start site of the RNA transcript, wherein the oligonucleotide comprises nucleotides linked by at least one modified internucleoside linkage or at least one bridged nucleotide.

43. A method of increasing THRB or NR1H4 gene expression in a cell, the method comprising delivering to a cell, expressing an RNA transcript of the THRB or NR1H4 gene, an oligonucleotide comprising two regions of complementarity each of which is complementary with at least 5 contiguous nucleotides of an RNA transcript, wherein the nucleotide at the 3′-end of the first region of complementary is complementary with a nucleotide within 100 nucleotides of the transcription start site of the RNA transcript and wherein the second region of complementarity is complementary with a region of the RNA transcript that ends within 300 nucleotides of the 3′-end of the RNA transcript.

44. The method of claim 42 or 43, wherein the RNA transcript is an mRNA transcript.

45. A method of increasing stability of an RNA transcript expressed by a THRB or NR1H4 gene in a cell, the method comprising delivering to the cell a first stabilizing oligonucleotide that targets a 5′ region of the RNA transcript and a second stabilizing oligonucleotide that targets the 3′ region of the RNA transcript.

46. The method of claim 45, wherein the first stabilizing oligonucleotide is covalently linked with the second stabilizing oligonucleotide.

47. The method of claim 45 or 46, wherein the first stabilizing oligonucleotide comprises a region of complementarity that is complementary with the RNA transcript at a position within 10 nucleotides of the first transcribed nucleotide at the 5′ end of the RNA transcript.

48. The method of any one of claims 45 to 47, wherein the RNA transcript comprises a 5′-methylguanosine cap, and wherein the first stabilizing oligonucleotide comprises a region of complementarity that is complementary with the RNA transcript at a position within 10 nucleotides of the nucleotide immediately internal to the 5′-methylguanosine cap.

49. The method any one of claims 45 to 48, wherein the second stabilizing oligonucleotide comprises a region of complementarity that is complementary with the RNA transcript at a position within 250 nucleotides of the 3′ end of the RNA transcript.

50. The method any one of claims 45 to 49, wherein the RNA transcript comprises a 3′-poly(A) tail, and wherein the second stabilizing oligonucleotide comprises a region of complementarity that is complementary with the RNA transcript at a position within 100 nucleotides of the polyadenylation junction of the RNA transcript.

51. The method any one of claims 45 to 50, wherein the region of complementarity of the second stabilizing oligonucleotide is immediately adjacent to or overlapping the polyadenylation junction of the RNA transcript.

52. The method of any one of claims 45 to 51, wherein the RNA transcript is an mRNA transcript.

53. The method of any one of claims 45 to 51, wherein the RNA transcript is an mRNA transcript of THRB or NR1H4 as provided in Table 1.

54. A method of increasing stability of an RNA transcript expressed by a THRB or NR1H4 gene in a cell, the method comprising delivering to the cell expressing the RNA transcript an oligonucleotide of any one of claims 63-104 that targets the RNA transcript, thereby increasing stability of the RNA transcript.

55. The method of any one of claims 45 to 54, wherein the cell is in vitro.

56. The method of any one of claims 45 to 54, wherein the cell is in vivo.

57. The method of any one of claims 54 to 56, wherein the RNA transcript is an mRNA transcript.

58. The method of any one of claims 54 to 56, wherein the RNA transcript is an mRNA transcript provided in Table 1.

59. A method of treating a condition or disease associated with decreased levels of an RNA transcript expressed from a THRB or NR1H4 gene in a subject, the method comprising administering an oligonucleotide of any one of claims 63-104 to the subject.

60. The method of any one of claim 45 to 56 or 59, wherein the RNA transcript is an mRNA.

61. The method of any one of claim 45 to 56 or 59, wherein the RNA transcript is a mRNA of THRB or NR1H4 as provided in Table 1.

62. The method of any one of claims 1 to 61, wherein the cell is a human liver cell.

63. An oligonucleotide of 8 to 50 nucleotides in length, the oligonucleotide comprising a region of complementarity that is complementary with at least 5 contiguous nucleotides of an RNA transcript expressed from a THRB or NR1H4 gene, wherein the nucleotide at the 3′-end of the region of complementary is complementary with a nucleotide within 10 nucleotides of the transcription start site of the RNA transcript, wherein the oligonucleotide comprises nucleotides linked by at least one modified internucleoside linkage or at least one bridged nucleotide.

64. An oligonucleotide comprising two regions of complementarity each of which is complementary with at least 5 contiguous nucleotides of an RNA transcript expressed from a THRB or NR1H4 gene, wherein the nucleotide at the 3′-end of the first region of complementary is complementary with a nucleotide within 100 nucleotides of the transcription start site of the RNA transcript and wherein the second region of complementarity is complementary with a region of the RNA transcript that ends within 300 nucleotides of the 3′-end of the RNA transcript.

65. An oligonucleotide comprising the general formula 5′-X₁-X₂-3′, wherein X₁ comprises 5 to 20 nucleotides that have a region of complementarity that is complementary with at least 5 contiguous nucleotides of an RNA transcript expressed from a THRB or NR1H4 gene, wherein the nucleotide at the 3′-end of the region of complementary of X₁ is complementary with the nucleotide at the transcription start site of the RNA transcript; and X₂ comprises 1 to 20 nucleotides.

66. The oligonucleotide of any one of claims 63 to 65, wherein the RNA transcript has a 7-methylguanosine cap at its 5′-end.

67. The oligonucleotide of claim 65, wherein the RNA transcript has a 7-methylguanosine cap, and wherein the nucleotide at the 3′-end of the region of complementary of X₁ is complementary with the nucleotide of the RNA transcript that is immediately internal to the 7-methylguanosine cap.

68. The oligonucleotide of claim 65, wherein at least the first nucleotide at the 5′-end of X₂ is a pyrimidine complementary with guanine.

69. The oligonucleotide of claim 68, wherein the second nucleotide at the 5′-end of X₂ is a pyrimidine complementary with guanine.

70. The oligonucleotide of claim 65, wherein X₂ comprises the formula 5′-Y₁-Y₂-Y₃-3′, wherein X₂ forms a stem-loop structure having a loop region comprising the nucleotides of Y₂ and a stem region comprising at least two contiguous nucleotides of Y₁ hybridized with at least two contiguous nucleotides of Y₃.

71. The oligonucleotide of claim 70, wherein Y₁, Y₂ and Y₃ independently comprise 1 to 10 nucleotides.

72. The oligonucleotide of claim 70 or 71, wherein Y₃ comprises, at a position immediately following the 3′-end of the stem region, a pyrimidine complementary with guanine.

73. The oligonucleotide of any one of claims 68 to 72, wherein the pyrimidine complementary with guanine is cytosine.

74. The oligonucleotide of claim 65, wherein X₂ comprises a region of complementarity that is complementary with at least 5 contiguous nucleotides of the RNA transcript that do not overlap the region of the RNA transcript that is complementary with the region of complementarity of X₁.

75. The oligonucleotide of claim 74, wherein the region of complementarity of X₂ is within 100 nucleotides of a polyadenylation junction of the RNA transcript.

76. The oligonucleotide of claim 75, wherein the region of complementarity of X₂ is complementary with the RNA transcript immediately adjacent to or overlapping the polyadenylation junction of the RNA transcript.

77. The oligonucleotide of claim 75 or 76, wherein X₂ further comprises at least 2 consecutive pyrimidine nucleotides complementary with adenine nucleotides of the poly(A) tail of the RNA transcript.

78. The oligonucleotide of any one of claims 63 to 77, wherein the RNA transcript is an mRNA.

79. The oligonucleotide of any one of claims 65 to 78, wherein the RNA transcript is an mRNA transcript, and wherein X₂ comprises a region of complementarity that is complementary with at least 5 contiguous nucleotides in the 3′-UTR of the transcript.

80. The oligonucleotide of any one of claims 63 to 79, wherein the RNA transcript is an mRNA of THRB or NR1H4 as provided in Table 1.

81. The oligonucleotide of claim 80, wherein X₂ comprises the sequence CC.

82. An oligonucleotide of 10 to 50 nucleotides in length having a first region complementary with at least 5 consecutive nucleotides of the 5′-UTR of an mRNA transcript expressed from a THRB or NR1H4 gene, and a second region complementary with at least 5 consecutive nucleotides of the 3′-UTR, poly(A) tail, or overlapping the polyadenylation junction of the mRNA transcript.

83. The oligonucleotide of claim 82, wherein the first of the at least 5 consecutive nucleotides of the 5′-UTR is within 10 nucleotides of the 5′-methylguanosine cap of the mRNA transcript.

84. The oligonucleotide of claim 82 or 83, wherein the second region is complementary with at least 5 consecutive nucleotides overlapping the polyadenylation junction.

85. The oligonucleotide of any one of claims 82 to 84, further comprising 2-20 nucleotides that link the 5′ end of the first region with the 3′ end of the second region.

86. The oligonucleotide of any one of claims 82 to 84, further comprising 2-20 nucleotides that link the 3′ end of the first region with the 5′ end of the second region.

87. The oligonucleotide of any one of claims 82 to 84, wherein the oligonucleotide is 10 to 50 nucleotide in length.

88. The oligonucleotide of any one of claims 82 to 84, wherein the oligonucleotide is 9 to 20 nucleotide in length.

89. The oligonucleotide of any one of claims 82 to 88, wherein the mRNA transcript is an mRNA transcript of THRB or NR1H4 provided in Table 1.

90. An oligonucleotide comprising the general formula 5′-X₁-X₂-3′, wherein
X₁ comprises 2 to 20 pyrimidine nucleotides that form base pairs with adenine; and X₂ comprises a region of complementarity that is complementary with at least 3 contiguous nucleotides of a poly-adenylated RNA transcript expressed from a THRB or NR1H4 gene, wherein the nucleotide at the 5′-end of the region of complementary of X₂ is complementary with the nucleotide of the RNA transcript that is immediately internal to the poly-adenylation junction of the RNA transcript.

91. The oligonucleotide of claim 90, wherein X₁ comprises 2 to 20 thymidines or uridines.

92. The oligonucleotide of claim 90 or 91, wherein the poly-adenylated RNA transcript is an mRNA.

93. The oligonucleotide of any one of claims 63 to 92, wherein the oligonucleotide comprises at least one modified internucleoside linkage.

94. The oligonucleotide of any one of claims 63 to 92, wherein the oligonucleotide comprises at least one modified nucleotide.

95. The oligonucleotide of any one of claims 63 to 94, wherein at least one nucleotide comprises a 2′ O-methyl.

96. The oligonucleotide of any one of claims 63 to 92, wherein the oligonucleotide comprises at least one ribonucleotide, at least one deoxyribonucleotide, at least one 2′-fluoro-deoxyribonucleotides or at least one bridged nucleotide.

97. The oligonucleotide of claim 96, wherein the bridged nucleotide is a LNA nucleotide, a cEt nucleotide or a ENA modified nucleotide.

98. The oligonucleotide of any one of claims 65 to 97, wherein each nucleotide of the oligonucleotide is a LNA nucleotide.

99. The oligonucleotide of any one of claims 65 to 98, wherein the nucleotides of the oligonucleotide comprise alternating deoxyribonucleotides and 2′-fluoro-deoxyribonucleotides, 2′-O-methyl nucleotides, or bridged nucleotides.

100. The oligonucleotide of any one of claims 65 to 93, wherein the oligonucleotide is mixmer.

101. The oligonucleotide of any one of claims 65 to 93, wherein the oligonucleotide is morpholino.

102. The oligonucleotide of any one of claims 65 to 101, wherein the cell is a human liver cell.

103. An oligonucleotide comprising a nucleotide sequence as set forth in Table 3.

104. An oligonucleotide comprising a fragment of at least 8 nucleotides of a nucleotide sequence as set forth in Table 3.

105. A composition comprising a first oligonucleotide having 5 to 25 nucleotides linked through internucleoside linkages, and a second oligonucleotide having 5 to 25 nucleotides linked through internucleoside linkages, wherein the first oligonucleotide is complementary with at least 5 consecutive nucleotides within 100 nucleotides of the 5′-end of an RNA transcript expressed from a THRB or NR1H4 gene and wherein the second oligonucleotide is complementary with at least 5 consecutive nucleotides within 100 nucleotides of the 3′-end of the RNA transcript.

106. The composition of claim 105, wherein the first oligonucleotide and second oligonucleotide are joined by a linker that is not an oligonucleotide having a sequence complementary with the RNA transcript.

107. The composition of claim 106, wherein the linker is an oligonucleotide.

108. The composition of claim 106, wherein the linker is a polypeptide.

109. The composition of claim 105, wherein the RNA transcript is an mRNA.

110. A composition comprising a plurality of oligonucleotides, wherein each of at least 75% of the oligonucleotides is an oligonucleotide selected from any one of claims 63 to 104.

111. The composition of claim 110, wherein the oligonucleotides are complexed with a monovalent cation.

112. The composition of claim 110 or 111, wherein the oligonucleotides are in a lyophilized form.

113. The composition of claim 110 or 111, wherein the oligonucleotides are in an aqueous solution.

114. A composition comprising an oligonucleotide of any one of claims 63 to 104 and a carrier.

115. A composition comprising an oligonucleotide of any one of claims 63 to 104 in a buffered solution.

116. A composition of comprising an oligonucleotide of any one of claims 63 to 104 conjugated to the carrier.

117. The composition of claim 116, wherein the carrier is a peptide.

118. The composition of claim 116, wherein the carrier is a steroid.

119. A pharmaceutical composition comprising an oligonucleotide of any one of claims 63 to 104 and a pharmaceutically acceptable carrier.

120. A kit comprising a container housing the composition of any one of claims 110 to 119.

121. A method of increasing gene expression in a liver cell in a human subject, the method comprising:
delivering to a human subject an oligonucleotide in an amount effective to increase gene expression in a liver cell of the subject, the oligonucleotide comprising the general formula 5′-X₁-X₂-3′, wherein X₁ comprises 5 to 20 nucleotides that have a region of complementarity that is complementary with at least 5 contiguous nucleotides of an mRNA transcript encoded by the gene, wherein the nucleotide at the 3′-end of the region of complementary of X₁ is complementary with the nucleotide at the transcription start site of the mRNA transcript; and X₂ comprises 1 to 20 nucleotides.

122. The method of claim 121, wherein the mRNA transcript has a 7-methylguanosine cap at its 5′-end.

123. The method of claim 121, wherein the RNA transcript has a 7-methylguanosine cap, and wherein the nucleotide at the 3′-end of the region of complementary of X₁ is complementary with the nucleotide of the RNA transcript that is immediately internal to the 7-methylguanosine cap.

124. The method of claim 121, wherein at least the first nucleotide at the 5′-end of X₂ is a pyrimidine complementary with guanine.

125. The method of claim 122, wherein the second nucleotide at the 5′-end of X₂ is a pyrimidine complementary with guanine.

126. The method of claim 121, wherein X₂ comprises the formula 5′-Y₁-Y₂-Y₃-3′, wherein X₂ forms a stem-loop structure having a loop region comprising the nucleotides of Y₂ and a stem region comprising at least two contiguous nucleotides of Y₁ hybridized with at least two contiguous nucleotides of Y₃.

127. The method of claim 126, wherein Y₁, Y₂ and Y₃ independently comprise 1 to 10 nucleotides.

128. The method of claim 126 or 127, wherein Y₃ comprises, at a position immediately following the 3′-end of the stem region, a pyrimidine complementary with guanine.

129. The method of any one of claims 122 to 128, wherein the pyrimidine complementary with guanine is cytosine.

130. The method of claim 121, wherein X₂ comprises a region of complementarity that is complementary with at least 5 contiguous nucleotides of the mRNA transcript that do not overlap the region of the mRNA transcript that is complementary with the region of complementarity of X₁.

131. The method of claim 130, wherein the region of complementarity of X₂ is within 100 nucleotides of a polyadenylation junction of the mRNA transcript.

132. The method of claim 131, wherein the region of complementarity of X₂ is complementary with the mRNA transcript immediately adjacent to or overlapping the polyadenylation junction of the mRNA transcript.

133. The method of claim 131 or 132, wherein X₂ further comprises at least 2 consecutive pyrimidine nucleotides complementary with adenine nucleotides of the poly(A) tail of the mRNA transcript.

134. The method of any one of claims 121 to 133, wherein X₂ comprises a region of complementarity that is complementary with at least 5 contiguous nucleotides in the 3′-UTR of the transcript.

135. The method of any one of claims 121 to 134, the delivery results in an increase in the level of a protein encoded by the mRNA.

136. The method of claim 135, wherein the increase in the level of the protein encoded by the mRNA is at least a 50% increase compared with an appropriate control cell to which the oligonucleotide was not delivered.

137. The method of claim 121, wherein X₂ comprises the sequence CC.

138. A method of increasing gene expression in a liver cell in a human subject, the method comprising:
delivering to a human subject an oligonucleotide in an amount effective to increase gene expression in a liver cell of the subject, the oligonucleotide being 10 to 50 nucleotides in length having a first region complementary with at least 5 consecutive nucleotides of the 5′-UTR of an mRNA transcript encoded by the gene, and a second region complementary with at least 5 consecutive nucleotides of the 3′-UTR, poly(A) tail, or overlapping the polyadenylation junction of the mRNA transcript.

139. The method of claim 138, wherein the first of the at least 5 consecutive nucleotides of the 5′-UTR is within 10 nucleotides of the 5′-methylguanosine cap of the mRNA transcript.

140. The method of claim 138 or 139, wherein the second region is complementary with at least 5 consecutive nucleotides overlapping the polyadenylation junction.

141. The method of any one of claims 138 to 140, further comprising 2-20 nucleotides that link the 5′ end of the first region with the 3′ end of the second region.

142. The method of any one of claims 138 to 140, further comprising 2-20 nucleotides that link the 3′ end of the first region with the 5′ end of the second region.

143. The method of any one of claims 138 to 142, wherein the oligonucleotide is 10 to 50 nucleotide in length.

144. The method of any one of claims 138 to 142, wherein the oligonucleotide is 9 to 20 nucleotide in length.

145. The method of any one of claims 138 to 144, wherein the gene is selected from the group consisting of THRB, HAMP, APOA1 and NR1H4.

146. A method of increasing gene expression in a liver cell in a human subject, the method comprising:
delivering to a human subject an oligonucleotide in an amount effective to increase gene expression in a liver cell of the subject, the oligonucleotide comprising the general formula 5′-X₁-X₂-3′, wherein X₁ comprises 2 to 20 pyrimidine nucleotides that form base pairs with adenine; and X₂ comprises a region of complementarity that is complementary with at least 3 contiguous nucleotides of a poly-adenylated RNA transcript encoded by the gene, wherein the nucleotide at the 5′-end of the region of complementary of X₂ is complementary with the nucleotide of the RNA transcript that is immediately internal to the poly-adenylation junction of the RNA transcript.

147. The method of claim 146, wherein X₁ comprises 2 to 20 thymidines or uridines.

148. The method of claim 146 or 147, wherein the poly-adenylated RNA transcript is an mRNA.

149. The method of any one of claims 121 to 148, wherein the oligonucleotide comprises at least one modified internucleoside linkage.

150. The method of any one of claims 121 to 149, wherein the oligonucleotide comprises at least one modified nucleotide.

151. The method of any one of claims 121 to 150, wherein at least one nucleotide comprises a 2′ O-methyl.

152. The method of any one of claims 121 to 151, wherein the oligonucleotide comprises at least one ribonucleotide, at least one deoxyribonucleotide, at least one 2′-fluoro-deoxyribonucleotides or at least one bridged nucleotide.

153. The method of claim 152, wherein the bridged nucleotide is a LNA nucleotide, a cEt nucleotide or a ENA modified nucleotide.

154. The method of any one of claims 121 to 153, wherein each nucleotide of the oligonucleotide is a LNA nucleotide.

155. The method of any one of claims 121 to 154, wherein the nucleotides of the oligonucleotide comprise alternating deoxyribonucleotides and 2′-fluoro-deoxyribonucleotides, 2′-O-methyl nucleotides, or bridged nucleotides.

156. The method of any one of claims 121 to 155, wherein the oligonucleotide is mixmer.

157. The method of any one of claims 121 to 156, wherein the oligonucleotide is morpholino.

158. The method of any one of claims 138 to 157, wherein the gene is selected from the group consisting of THRB, HAMP, APOA1 and NR1H4.

159. A method of increasing gene expression in a liver cell of a human subject, the method comprising:
delivering to a human subject an oligonucleotide in an amount effective to increase gene expression in a liver cell of the subject, the oligonucleotide being 8 to 50 nucleotides in length, the oligonucleotide comprising a region of complementarity that is complementary with at least 5 contiguous nucleotides of an mRNA transcript expressed from the gene, wherein the nucleotide at the 3′-end of the region of complementary is complementary with a nucleotide within 10 nucleotides of the transcription start site of the mRNA transcript, wherein the oligonucleotide comprises nucleotides linked by at least one modified internucleoside linkage or at least one bridged nucleotide.

160. A method of increasing gene expression in a liver cell of a human subject, the method comprising:
delivering to a human subject an oligonucleotide in an amount effective to increase gene expression in a liver cell of the subject, the oligonucleotide comprising two regions of complementarity each of which is complementary with at least 5 contiguous nucleotides of an mRNA transcript expressed from the gene, wherein the nucleotide at the 3′-end of the first region of complementary is complementary with a nucleotide within 100 nucleotides of the transcription start site of the mRNA transcript and wherein the second region of complementarity is complementary with a region of the mRNA transcript that ends within 300 nucleotides of the 3′-end of the mRNA transcript.

161. The method of claim 159 or 160, wherein the gene is selected from the group consisting of THRB, HAMP, APOA1 and NR1H4.

162. A method of increasing stability of an mRNA transcript in a liver cell in a human subject, the method comprising:
delivering to a human subject a first stabilizing oligonucleotide and a second stabilizing nucleotide in an amount effective to increase gene expression in a liver cell of the subject, the first stabilizing oligonucleotide targeting a 5′ region of an mRNA transcript expressed from the gene and a second stabilizing oligonucleotide that targets the 3′ region of the mRNA transcript.

163. The method of claim 162, wherein the first stabilizing oligonucleotide is covalently linked with the second stabilizing oligonucleotide.

164. The method of claim 162 or 163, wherein the first stabilizing oligonucleotide comprises a region of complementarity that is complementary with the mRNA transcript at a position within 10 nucleotides of the first transcribed nucleotide at the 5′ end of the mRNA transcript.

165. The method of any one of claims 162 to 164, wherein the mRNA transcript comprises a 5′-methylguanosine cap, and wherein the first stabilizing oligonucleotide comprises a region of complementarity that is complementary with the mRNA transcript at a position within 10 nucleotides of the nucleotide immediately internal to the 5′-methylguanosine cap.

166. The method any one of claims 162 to 165, wherein the second stabilizing oligonucleotide comprises a region of complementarity that is complementary with the mRNA transcript at a position within 250 nucleotides of the 3′ end of the mRNA transcript.

167. The method any one of claims 162 to 166, wherein the mRNA transcript comprises a 3′-poly(A) tail, and wherein the second stabilizing oligonucleotide comprises a region of complementarity that is complementary with the mRNA transcript at a position within 100 nucleotides of the polyadenylation junction of the mRNA transcript.

168. The method any one of claims 162 to 167, wherein the region of complementarity of the second stabilizing oligonucleotide is immediately adjacent to or overlapping the polyadenylation junction of the mRNA transcript.

169. The method of any one of claims 162 to 168, wherein the gene is selected from the group consisting of THRB, HAMP, APOA1 and NR1H4.

170. The method of any one of claims 121 to 137, wherein the gene is selected from the group consisting of THRB, HAMP, APOA1 and NR1H4.


SEQUENCE LISTING (125 sequences)

SEQ ID NO: 1 - DNA, 15 bp, Homo sapiens
ctgttataagctttt

SEQ ID NO: 2 - DNA, 15 bp, Homo sapiens
gttataagctttttc

SEQ ID NO: 3 - DNA, 15 bp, Homo sapiens
tatctgttataagct

SEQ ID NO: 4 - DNA, 15 bp, Homo sapiens
agtagatgtttattt

SEQ ID NO: 5 - DNA, 15 bp, Homo sapiens
taggcaaaggaatag

SEQ ID NO: 6 - DNA, 15 bp, Homo sapiens
ggtaggcaaaggaat

SEQ ID NO: 7 - DNA, 15 bp, Homo sapiens
ggcaaaggaatagtt

SEQ ID NO: 8 - DNA, 15 bp, Homo sapiens
gaaatgacacccagt

SEQ ID NO: 9 - DNA, 15 bp, Homo sapiens
aaatgacacccagta

SEQ ID NO: 10 - DNA, 15 bp, Homo sapiens
ggcaatggaatgaaa

SEQ ID NO: 11 - DNA, 15 bp, Homo sapiens
caatggaatgaaatg

SEQ ID NO: 12 - DNA, 15 bp, Homo sapiens
atggaatgaaatgac

SEQ ID NO: 13 - DNA, 15 bp, Homo sapiens
gtttcaagtacccgc

SEQ ID NO: 14 - DNA, 15 bp, Homo sapiens
gaccggagaacgaaa

SEQ ID NO: 15 - DNA, 15 bp, Homo sapiens
ctttggaaggtgttt

SEQ ID NO: 16 - DNA, 15 bp, Homo sapiens
tttctttggaaggtg

SEQ ID NO: 17 - DNA, 15 bp, Homo sapiens
agttaatccccgccg

SEQ ID NO: 18 - DNA, 15 bp, Homo sapiens
tcctgcaaaatgtca

SEQ ID NO: 19 - DNA, 15 bp, Homo sapiens
ccccgcagtctccac

SEQ ID NO: 20 - DNA, 15 bp, Homo sapiens
tctccaccctcctcc

SEQ ID NO: 21 - DNA, 15 bp, Homo sapiens
gagcgccggcgactg

SEQ ID NO: 22 - DNA, 15 bp, Homo sapiens
aggatgtgcgccttc

SEQ ID NO: 23 - DNA, 14 bp, Homo sapiens
ggcgcagcgaggaa

SEQ ID NO: 24 - DNA, 15 bp, Homo sapiens
tttcactgacatctc

SEQ ID NO: 25 - DNA, 15 bp, Homo sapiens
ggtggtctgagcccc

SEQ ID NO: 26 - DNA, 15 bp, Homo sapiens
gggcctgccagggga

SEQ ID NO: 27 - DNA, 15 bp, Homo sapiens
accgagtgacagtcg

SEQ ID NO: 28 - DNA, 15 bp, Homo sapiens
gtctgggaccgagtg

SEQ ID NO: 29 - DNA, 15 bp, Homo sapiens
tgggaccgagtgaca

SEQ ID NO: 30 - DNA, 15 bp, Homo sapiens
gcagaggtgtgttca

SEQ ID NO: 31 - DNA, 15 bp, Homo sapiens
cggcagaggtgtgtt

SEQ ID NO: 32 - DNA, 15 bp, Homo sapiens
gggcagacggggtca

SEQ ID NO: 33 - DNA, 15 bp, Homo sapiens
ctctggtttggaaaa

SEQ ID NO: 34 - DNA, 15 bp, Homo sapiens
ctggtttggaaaaca

SEQ ID NO: 35 - DNA, 15 bp, Homo sapiens
gtttggaaaacaaaa

SEQ ID NO: 36 - DNA, 15 bp, Homo sapiens
ggaaaacaaaagaac

SEQ ID NO: 37 - DNA, 15 bp, Homo sapiens
gaaaacaaaagaacc

SEQ ID NO: 38 - DNA, 15 bp, Homo sapiens
tttggaaaacaaaag

SEQ ID NO: 39 - DNA, 15 bp, Homo sapiens
tggaaaacaaaagaa

SEQ ID NO: 40 - DNA, 15 bp, Homo sapiens
tctggggcagcagga

SEQ ID NO: 41 - DNA, 15 bp, Homo sapiens
atagaaaggaacctt

SEQ ID NO: 42 - DNA, 15 bp, Homo sapiens
tagaaaggaaccttg

SEQ ID NO: 43 - DNA, 15 bp, Homo sapiens
attaacaatccttcc

SEQ ID NO: 44 - DNA, 15 bp, Homo sapiens
cttccctgctaaatg

SEQ ID NO: 45 - DNA, 15 bp, Homo sapiens
ccctgctaaatgata

SEQ ID NO: 46 - DNA, 15 bp, Homo sapiens
tgatataaacataga

SEQ ID NO: 47 - DNA, 15 bp, Homo sapiens
tccccgggactgaac

SEQ ID NO: 48 - DNA, 16 bp, Homo sapiens
tacaacttctttgaat

SEQ ID NO: 49 - DNA, 15 bp, Homo sapiens
tctatcacttccccg

SEQ ID NO: 50 - DNA, 15 bp, Homo sapiens
tcctgggcacccgta

SEQ ID NO: 51 - DNA, 15 bp, Homo sapiens
tttctgtacacatca

SEQ ID NO: 52 - DNA, 15 bp, Homo sapiens
acagccactgaaaat

SEQ ID NO: 53 - DNA, 15 bp, Homo sapiens
ttcacagccactgaa

SEQ ID NO: 54 - DNA, 15 bp, Homo sapiens
taaagaaatgagttt

SEQ ID NO: 55 - DNA, 15 bp, Homo sapiens
acatttaaagaaatg

SEQ ID NO: 56 - DNA, 15 bp, Homo sapiens
aagaaatgagtttgt

SEQ ID NO: 57 - DNA, 15 bp, Homo sapiens
tgccattatgtttgc

SEQ ID NO: 58 - DNA, 15 bp, Homo sapiens
tcctgttgccattat

SEQ ID NO: 59 - DNA, 15 bp, Homo sapiens
gaaaatcctgttgcc

SEQ ID NO: 60 - DNA, 15 bp, Homo sapiens
ctgttgccattatgt

SEQ ID NO: 61 - DNA, 15 bp, Homo sapiens
tagaattgaagtaac

SEQ ID NO: 62 - DNA, 15 bp, Homo sapiens
tgaagtaacaatcaa

SEQ ID NO: 63 - DNA, 15 bp, Homo sapiens
aagtaacaatcaatt

SEQ ID NO: 64 - DNA, 15 bp, Homo sapiens
tcatcaagatttctt

SEQ ID NO: 65 - DNA, 15 bp, Homo sapiens
tatctagcccaatat

SEQ ID NO: 66 - DNA, 15 bp, Homo sapiens
ttctatctagcccaa

SEQ ID NO: 67 - DNA, 33 bp, Homo sapiens
ctccgccctccagtttttattattttgcttttt

SEQ ID NO: 68 - DNA, 31 bp, Homo sapiens
ccgccctccagtttttattattttgcttttt

SEQ ID NO: 69 - DNA, 29 bp, Homo sapiens
gccctccagtttttattattttgcttttt

SEQ ID NO: 70 - DNA, 33 bp, Homo sapiens
cgctccgccctccagtttttattattttgcttt

SEQ ID NO: 71 - DNA, 31 bp, Homo sapiens
cgctccgccctccagtttttattattttgct

SEQ ID NO: 72 - DNA, 29 bp, Homo sapiens
cgctccgccctccagtttttattattttg

SEQ ID NO: 73 - DNA, 31 bp, Homo sapiens
ctccgccctccagtttttattattttgcttt

SEQ ID NO: 74 - DNA, 27 bp, Homo sapiens
ccgccctccagtttttattattttgct

SEQ ID NO: 75 - DNA, 25 bp, Homo sapiens
gccctccagtttttattattttgct

SEQ ID NO: 76 - DNA, 23 bp, Homo sapiens
ccctccagtttttattattttgc

SEQ ID NO: 77 - DNA, 21 bp, Homo sapiens
cctccagtttttattattttg

SEQ ID NO: 78 - DNA, 29 bp, Homo sapiens
gctccgccctccagattattttgcttttt

SEQ ID NO: 79 - DNA, 27 bp, Homo sapiens
tccgccctccagattattttgcttttt

SEQ ID NO: 80 - DNA, 25 bp, Homo sapiens
cgccctccagattattttgcttttt

SEQ ID NO: 81 - DNA, 23 bp, Homo sapiens
ccctccagattattttgcttttt

SEQ ID NO: 82 - DNA, 27 bp, Homo sapiens
gctccgccctccagattattttgcttt

SEQ ID NO: 83 - DNA, 25 bp, Homo sapiens
gctccgccctccagattattttgct

SEQ ID NO: 84 - DNA, 23 bp, Homo sapiens
gctccgccctccagattattttg

SEQ ID NO: 85 - DNA, 25 bp, Homo sapiens
tccgccctccagattattttgcttt

SEQ ID NO: 86 - DNA, 21 bp, Homo sapiens
cgccctccagattattttgct

SEQ ID NO: 87 - DNA, 19 bp, Homo sapiens
gccctccagattattttgc

SEQ ID NO: 88 - DNA, 17 bp, Homo sapiens
ccctccagattattttg

SEQ ID NO: 89 - DNA, 15 bp, Homo sapiens
ctccagattattttg

SEQ ID NO: 90 - DNA, 35 bp, Homo sapiens
cgctccgccctccagtttttattattttgcttttt

SEQ ID NO: 91 - DNA, 28 bp, Mus musculus
agttcaaggatcagccattttggaaagg

SEQ ID NO: 92 - DNA, 25 bp, Mus musculus
tcaaggatcagccattttggaaagg

SEQ ID NO: 93 - DNA, 23 bp, Mus musculus
aaggatcagccattttggaaagg

SEQ ID NO: 94 - DNA, 21 bp, Mus musculus
ggatcagccattttggaaagg

SEQ ID NO: 95 - DNA, 25 bp, Mus musculus
agttcaaggatcagccattttggaa

SEQ ID NO: 96 - DNA, 23 bp, Mus musculus
agttcaaggatcagccattttgg

SEQ ID NO: 97 - DNA, 27 bp, Mus musculus
gttcaaggatcagccattttggaaagg

SEQ ID NO: 98 - DNA, 23 bp, Mus musculus
tcaaggatcagccattttggaaa

SEQ ID NO: 99 - DNA, 21 bp, Mus musculus
aaggatcagccattttggaaa

SEQ ID NO: 100 - DNA, 19 bp, Mus musculus
aggatcagccattttggaa

SEQ ID NO: 101 - DNA, 17 bp, Mus musculus
ggatcagccattttgga

SEQ ID NO: 102 - DNA, 28 bp, Mus musculus
ctccgacagtctgccattttggaaaggt

SEQ ID NO: 103 - DNA, 25 bp, Mus musculus
cgacagtctgccattttggaaaggt

SEQ ID NO: 104 - DNA, 23 bp, Mus musculus
acagtctgccattttggaaaggt

SEQ ID NO: 105 - DNA, 25 bp, Mus musculus
ctccgacagtctgccattttggaaa

SEQ ID NO: 106 - DNA, 23 bp, Mus musculus
ctccgacagtctgccattttgga

SEQ ID NO: 107 - DNA, 21 bp, Mus musculus
ctccgacagtctgccattttg

SEQ ID NO: 108 - DNA, 27 bp, Mus musculus
ctccgacagtctgccattttggaaagg

SEQ ID NO: 109 - DNA, 23 bp, Mus musculus
ccgacagtctgccattttggaaa

SEQ ID NO: 110 - DNA, 19 bp, Mus musculus
gacagtctgccattttgga

SEQ ID NO: 111 - DNA, 17 bp, Mus musculus
acagtctgccattttgg

SEQ ID NO: 112 - DNA, 30 bp, Mus musculus
cggagctctccgacacattttggaaaggtt

SEQ ID NO: 113 - DNA, 29 bp, Mus musculus
ggagctctccgacacattttggaaaggtt

SEQ ID NO: 114 - DNA, 25 bp, Mus musculus
agctctccgacacattttggaaagg

SEQ ID NO: 115 - DNA, 21 bp, Mus musculus
ctctccgacacattttggaaa

SEQ ID NO: 116 - DNA, 19 bp, Mus musculus
tctccgacacattttggaa

SEQ ID NO: 117 - DNA, 17 bp, Mus musculus
ctccgacacattttgga

SEQ ID NO: 118 - DNA, 15 bp, Mus musculus
tccgacacattttgg

SEQ ID NO: 119 - DNA, 15 bp, Homo sapiens
cgctccgccctccag

SEQ ID NO: 120 - DNA, 15 bp, Homo sapiens
attattttgcttttt

SEQ ID NO: 121 - DNA, 18 bp, Artificial Sequence
  Synthetic Polynucleotide
ttttttttttggttttcc

SEQ ID NO: 122 - DNA, 29 bp, Artificial Sequence
  Synthetic Polynucleotide
aaaaaaaaaaaaaaaaaaaaaaaaaaaaa

SEQ ID NO: 123 - DNA, 11 bp, Artificial Sequence
  Synthetic Polynucleotide
aaaaaaaaaaa

SEQ ID NO: 124 - DNA, 15 bp, Mus musculus
agttcaaggatcagc

SEQ ID NO: 125 - DNA, 13 bp, Mus musculus
cattttggaaagg